SEQUENCE LISTING (55 sequences)

SEQ ID NO: 1 - DNA, 5984 bp, Bovine
  misc_feature (1294)..(1294): n is a, c, g, or t
gctgtcatcactgcatcagcatcattattctgtggaaggaattccacacagtgagttgtgagaccctagactagatcacacagtaaattcattgttaccttgctgctgctgctaagtcgcctcagtcgtgtccgactctgtgcaaccccatagacggcagcccaccaggctcccctgtccctgggattccccagacaagaacactggagtgggttgccatttccttctccaatgcatgaaagtgaaaagtgaaagtgaagtcgctcagtcatgtccgactcttagcttaggagagctttaaaaaggagtacctgcctccttattgtttaggttaaacttagagtggcaaaacggggcaggggttgaaggaaattatctcagtggtttatactgatgcttatgtgtggacagggaagcctggtatactgcagtccatggggttgcaaagagtaggacacaactgagcgactggaatgatgtcctgtactctcagattttggaacttccatggacgacccatgaggagacaggttggacggctggcagtcatcattgtgggcgcccttgttagtaagctccttgaaggtagggataaagatgtttttgttttgtgtccctggcacctgagacaaggtgtgatccacaacacctcttcagtaaacattgattaaatcagtcaaccccagcaacagggatttaggttgctggtaggaggtcacaggtatccttgtttgggatagcctagactggacatggaacaacagactggttccaaataggaaaaggagtacatcaaggctgtatattgtcaccctgcttatttaacttatatgcagagtacatcatgagaaacgctggactggaagaaacacaagctggaatcaagattgccgggagaaatatcaataacctcagatatgcagatgacaccacccttatggcagaaagtgaagaggaactcaaaagcctcttgatgaaagtgaaagtggagagtggaaaagttggcttaaagctcaacattcagaaaatgaagatcatggcatccggtcccatcacttcatgggaaatagatggggaaacagtggaaacagtgtcagactttattttggggggctccaaaatcactacagatggtgactgcagccatgaaattaaaagatgcttactccttggaagaaaagttatggccaacctagatagcatattaaaaagcagagacattactttgccaacaaaggtccgtctagtcaaggctatgggttttcctgtggtcatgtatggatgtgagagtggnactgtgaagaangctgagcgccgaagaaatgatgcttttgaactgtgggtgttggaacctcatgcgaagagtggactcattggaaaagactctgatgctgggaggatttgggggcaggaggagaaggggacgacagaggatgagatggctggatggcatcactgactcaatggacgtgagtctgggtgaactccgggagttggtgatggacagggaggcctggcgtgctgcaattcctggggtcacaaagagtcagacatgactgagcgactgaactgaactgaagctgaagatgggggcttccccgatggctcagcgggtaaaggatctgcctgcaatacaggagacgtgggttccatccctgggtcagaaaaatcccctggaaaaagaatggcagcccattccagtatccttgcccataaaatcctatggacagaggagcctggcgggatgcagtcattggggtcacagagtcggacaccactgagcacacgcacagcaggctggagctgcatagcaagagctgccagtaacataaagctttgtgtgtacactagtctcacctctttgagcttaatccagttcaacactacttgagcacctacttatgcaaggtacttaggaccaacttcacatgaaagtcaagtattttttaaaagaaaacctctgctaaataacaagtaagatactcttatcaataacatataattgataatatgttattattatgtttttccagttcttcacagaccacccaggtccctcttgggatcaccagcaaatactcagctccctgattgggcccaggagtccattgatttgctgccaccgtcctgtcctctgagaatactcataactgtcccaagactcggctccctgaatgccatcccagctcctgacccttgcatgttttctctttgtcactagattcagaatggaagccacaaaatgaaacatgctatttactctgactttgccgctctggttttcctcctgcctggtgcttgtttttcatttgactttttccaaaatgagaagagaaaagggctctagatttctcgaagagatcggacccttctaccttgagaatgcctttttgtaatgtctacattttaaaactatcaaactgaacagggcagattttaagtaggcttacagattaggatcattgcaatacattgcagctaaatatcagaactagatgatgtcagctctcatcggaatgaccaactaagaactgaagtgatgtaggaactacttgggtccctaacattcaagaggtaatattccatgagcagatgtcagttaacaatgttagccaacgagacatatcagttcctgagcatatgttggatgttagctgatcagtactgatattaatcatactttggacagtagacagtctgtaaacacccagttgacaccaaagaaaataactgatttgattttctgctattgctyttgaagccctgaccgtatctcttgtcatacttgagataagttcactgattatgttccatctacaccctgtatatacttctgtggttgggatttactgctttctattctaattatttgtttaccagttccatactagcctgtaagactgtgctggggttgtgctttattactgtttgccaggcctacaatggcataagtaggagcttgggagtaaaaaacgatttggatctgtgaagaaataactccagaaagaatgaagggatggagccaaagcaaaaacaataccaagttgtggatatgaccggtgatagaagaaaggtccagtgctgtaaagagcaatattgcataggaacctggaatgttagctccatgaatcaaggcaaattgaaagtggtcaaacaggagatggcaagagtgaacatcaacattctaggaatcagagaactaaaatggactggaatgggtgaatttaaatcagatgaccattatatctactactgagggcaggaatcccttagaagaaatagagtagccatcatggtcaacaaaagagtccaaaatgcagtacttggatgcaaatctcaaaacgacagaatgatctctgttcatttccaaggcaaaccattcaatatcacagtaatccaagtctatgccccaaccagtaatgctgaagaagctgaagttgaacagttctatgaagacctacaagaccttttagaactaacacccaaaaaagatgtccttttcattacaggggactggaatgctaaagtaggaagtcaagaaacacctggagtaacaggcaaatttggccttggaatatggaatgaagcagggcaaagactaatggagtttttccaagaaaatgcactggtcatagcaaacaccttcttccaataacacaagagaaaactctacacatggacatcaccagatggtcatcaccgaaatcagattgattatattctttgcagccaaagatggagaagctctataagtcagcaaaaacaagaccaggagctgactgtggctcagatcaggaactccttattaccaaattcagacttaaattgaagaaagtaggcaaaaccactagaccattcaggtatgacctaaatcaaatcccttatgattatacagtggaagtgagaaatacatttatgcgcctagatctgatagatagagtgcctgatgaactatggatggaggttcgtgacattgtataggagacagggatcaagaccatccccatggaaaagaaatgcagaaaagcaaaatggctgtctggggaggtcttacaaatagctgagaaaagaagagacgtgaaaagcaaaggagaaaaggaaagatataagcatctgaatgcagagttccaaagaaaggcacggagagataagaaagccttcctcagcgatcaatgcaaagaaatagagggaaataactgaatgggaaagattagagatctcttcaagaaaattagagacaccaagggaacatttcatgcaaagatgggctcgataaaggacagaaatggtatggacctaacagaagcagaagatattaagaagaggtagcaagaatacacagaagaactgtacaaaaaagatcttcacgacccagataatcacgatggtgtgatcactgacctagagccagacatcctggaatgtgaagtcaagtgggtcttagaaagcatcactacgaacaaagctagtggagatgatggaattccagttgagctatttcaaatcctaaaagatgatgctgtgaaagtgctgcactcaatatgccagcaaatttggacaactcagcagtggccacaggactggaaaaggtcagttttcattccaaccccaaagaaaggaattgccaaagaatgctcaaactaccgcacaattgcactcatctcacacgctagtaaagtaatgctcaaaatcctccaaatcaggctttagcaatacacgaactgtaaacttccagatgttcaagtaggttttagaaaaggcagaggaaccagagatcaaattgtcatcatccactggatcatggaaaaagcaagagagttctagaaaaacatctatttatgctttattgactatgccaaagccttcgactgtgtggatcacaataaactgtggaaaattctgaaagagatgggaataccagaccacctgacctgcctcttgagaaacctatatgcaggtcagaaagcaacagttagaacttggaacagactggttccaaataggaaaaggagcacatcaaggctctatattgtcaccctgcttatttaacttatatgcagagtacatcatgagaagtgatgggctggaagaagcacaagctggaatcaagattgccgggagaaatatcaataacctcagatatgcagatgacaccacccttaggcggaaggtgaggaggacttcaaaaccttctgtaggaagtggaaagtggggagtgaaaaagtgggtttaaggttcacctttcggaaatgtagattcagggcttcggttcccttcatttcaggggaattggagggggaaccggtggaaccgggttcggcttttttttgggtctcccagtggtcctgtatggatgtgagagttggactgtgaagaaagctgagtgcagaagaattgatgcttttgaactgtggtgttggagaagactcttgagagtcccttggactgcaaggagatccaaccagtccattctgaaggagatcagccctgggatttctttggaaggagtgatgctgaagctgaaactccagtactttggccacctcatgggaagagttgactcattggaaaatactctgatgctgggaaggattgggggcaggaggagaaggggaagacagagatgaaatggctggatggcatcactgactcgatggacatgagtttgagtgaactccgggagttagtgatggacagggagacctgcgtgctgtgattcatggggtcacaaagagttggacaggaccgagtgactgaactgaactgaatgcccctgacaggactagataacgtactcctttgtcattactgtaggtctaagatatgaaatgtttaccattggagacacactaaactattcttataaaagactgggggaggaaatcagtgacttacattccctacagcaataagagaataagtctgatggctatttcaccaagttctcagttttagggcactgacttggattgctttacatcatg

SEQ ID NO: 2 - DNA, 500 bp, Bovine
gctgtcatcactgcatcagcatcattattctgtggaaggaattccacacagtgagttgtgagaccctagactagatcacacagtaaattcattgttaccttgctgctgctgctaagtcgcctcagtcgtgtccgactctgtgcaaccccatagacggcagcccaccaggctcccctgtccctgggattccccagacaagaacactggagtgggttgccatttccttctccaatgcatgaaagtgaaaagtgaaagtgaagtcgctcagtcatgtccgactcttagcttaggagagctttaaaaaggagtacctgcctccttattgtttaggttaaacttagagtggcaaaacggggcaggggttgaaggaaattatctcagtggtttatactgatgcttatgtgtggacagggaagcctggtatactgcagtccatggggttgcaaagagtaggacacaactgagcgactggaatgatgtcctgtactctcagattttgg

SEQ ID NO: 3 - DNA, 100 bp, Bovine
aacttccatggacgacccatgaggagacaggttggacggctggcagtcatcattgtgggcgcccttgttagtaagctccttgaaggtagggataaagatg

SEQ ID NO: 4 - DNA, 1450 bp, Bovine
  misc_feature (694)..(694): n is a, c, g, or t
tttttgttttgtgtccctggcacctgagacaaggtgtgatccacaacacctcttcagtaaacattgattaaatcagtcaaccccagcaacagggatttaggttgctggtaggaggtcacaggtatccttgtttgggatagcctagactggacatggaacaacagactggttccaaataggaaaaggagtacatcaaggctgtatattgtcaccctgcttatttaacttatatgcagagtacatcatgagaaacgctggactggaagaaacacaagctggaatcaagattgccgggagaaatatcaataacctcagatatgcagatgacaccacccttatggcagaaagtgaagaggaactcaaaagcctcttgatgaaagtgaaagtggagagtggaaaagttggcttaaagctcaacattcagaaaatgaagatcatggcatccggtcccatcacttcatgggaaatagatggggaaacagtggaaacagtgtcagactttattttggggggctccaaaatcactacagatggtgactgcagccatgaaattaaaagatgcttactccttggaagaaaagttatggccaacctagatagcatattaaaaagcagagacattactttgccaacaaaggtccgtctagtcaaggctatgggttttcctgtggtcatgtatggatgtgagagtggnactgtgaagaangctgagcgccgaagaaatgatgcttttgaactgtgggtgttggaacctcatgcgaagagtggactcattggaaaagactctgatgctgggaggatttgggggcaggaggagaaggggacgacagaggatgagatggctggatggcatcactgactcaatggacgtgagtctgggtgaactccgggagttggtgatggacagggaggcctggcgtgctgcaattcctggggtcacaaagagtcagacatgactgagcgactgaactgaactgaagctgaagatgggggcttccccgatggctcagcgggtaaaggatctgcctgcaatacaggagacgtgggttccatccctgggtcagaaaaatcccctggaaaaagaatggcagcccattccagtatccttgcccataaaatcctatggacagaggagcctggcgggatgcagtcattggggtcacagagtcggacaccactgagcacacgcacagcaggctggagctgcatagcaagagctgccagtaacataaagctttgtgtgtacactagtctcacctctttgagcttaatccagttcaacactacttgagcacctacttatgcaaggtacttaggaccaacttcacatgaaagtcaagtattttttaaaagaaaacctctgctaaataacaagtaagatactcttatcaataacatataattgataatatgttattattatgtttttccagttcttcacagaccaccc

SEQ ID NO: 5 - DNA, 950 bp, Bovine
aggtccctcttgggatcaccagcaaatactcagctccctgattgggcccaggagtccattgatttgctgccaccgtcctgtcctctgagaatactcataactgtcccaagactcggctccctgaatgccatcccagctcctgacccttgcatgttttctctttgtcactagattcagaatggaagccacaaaatgaaacatgctatttactctgactttgccgctctggttttcctcctgcctggtgcttgtttttcatttgactttttccaaaatgagaagagaaaagggctctagatttctcgaagagatcggacccttctaccttgagaatgcctttttgtaatgtctacattttaaaactatcaaactgaacagggcagattttaagtaggcttacagattaggatcattgcaatacattgcagctaaatatcagaactagatgatgtcagctctcatcggaatgaccaactaagaactgaagtgatgtaggaactacttgggtccctaacattcaagaggtaatattccatgagcagatgtcagttaacaatgttagccaacgagacatatcagttcctgagcatatgttggatgttagctgatcagtactgatattaatcatactttggacagtagacagtctgtaaacacccagttgacaccaaagaaaataactgatttgattttctgctattgctyttgaagccctgaccgtatctcttgtcatacttgagataagttcactgattatgttccatctacaccctgtatatacttctgtggttgggatttactgctttctattctaattatttgtttaccagttccatactagcctgtaagactgtgctggggttgtgctttattactgtttgccaggcctacaatggcataagtaggagcttgggagtaaaaaacgatttggatctgtgaagaaataactc

SEQ ID NO: 6 - DNA, 2984 bp, Bovine
cagaaagaatgaagggatggagccaaagcaaaaacaataccaagttgtggatatgaccggtgatagaagaaaggtccagtgctgtaaagagcaatattgcataggaacctggaatgttagctccatgaatcaaggcaaattgaaagtggtcaaacaggagatggcaagagtgaacatcaacattctaggaatcagagaactaaaatggactggaatgggtgaatttaaatcagatgaccattatatctactactgagggcaggaatcccttagaagaaatagagtagccatcatggtcaacaaaagagtccaaaatgcagtacttggatgcaaatctcaaaacgacagaatgatctctgttcatttccaaggcaaaccattcaatatcacagtaatccaagtctatgccccaaccagtaatgctgaagaagctgaagttgaacagttctatgaagacctacaagaccttttagaactaacacccaaaaaagatgtccttttcattacaggggactggaatgctaaagtaggaagtcaagaaacacctggagtaacaggcaaatttggccttggaatatggaatgaagcagggcaaagactaatggagtttttccaagaaaatgcactggtcatagcaaacaccttcttccaataacacaagagaaaactctacacatggacatcaccagatggtcatcaccgaaatcagattgattatattctttgcagccaaagatggagaagctctataagtcagcaaaaacaagaccaggagctgactgtggctcagatcaggaactccttattaccaaattcagacttaaattgaagaaagtaggcaaaaccactagaccattcaggtatgacctaaatcaaatcccttatgattatacagtggaagtgagaaatacatttatgcgcctagatctgatagatagagtgcctgatgaactatggatggaggttcgtgacattgtataggagacagggatcaagaccatccccatggaaaagaaatgcagaaaagcaaaatggctgtctggggaggtcttacaaatagctgagaaaagaagagacgtgaaaagcaaaggagaaaaggaaagatataagcatctgaatgcagagttccaaagaaaggcacggagagataagaaagccttcctcagcgatcaatgcaaagaaatagagggaaataactgaatgggaaagattagagatctcttcaagaaaattagagacaccaagggaacatttcatgcaaagatgggctcgataaaggacagaaatggtatggacctaacagaagcagaagatattaagaagaggtagcaagaatacacagaagaactgtacaaaaaagatcttcacgacccagataatcacgatggtgtgatcactgacctagagccagacatcctggaatgtgaagtcaagtgggtcttagaaagcatcactacgaacaaagctagtggagatgatggaattccagttgagctatttcaaatcctaaaagatgatgctgtgaaagtgctgcactcaatatgccagcaaatttggacaactcagcagtggccacaggactggaaaaggtcagttttcattccaaccccaaagaaaggaattgccaaagaatgctcaaactaccgcacaattgcactcatctcacacgctagtaaagtaatgctcaaaatcctccaaatcaggctttagcaatacacgaactgtaaacttccagatgttcaagtaggttttagaaaaggcagaggaaccagagatcaaattgtcatcatccactggatcatggaaaaagcaagagagttctagaaaaacatctatttatgctttattgactatgccaaagccttcgactgtgtggatcacaataaactgtggaaaattctgaaagagatgggaataccagaccacctgacctgcctcttgagaaacctatatgcaggtcagaaagcaacagttagaacttggaacagactggttccaaataggaaaaggagcacatcaaggctctatattgtcaccctgcttatttaacttatatgcagagtacatcatgagaagtgatgggctggaagaagcacaagctggaatcaagattgccgggagaaatatcaataacctcagatatgcagatgacaccacccttaggcggaaggtgaggaggacttcaaaaccttctgtaggaagtggaaagtggggagtgaaaaagtgggtttaaggttcacctttcggaaatgtagattcagggcttcggttcccttcatttcaggggaattggagggggaaccggtggaaccgggttcggcttttttttgggtctcccagtggtcctgtatggatgtgagagttggactgtgaagaaagctgagtgcagaagaattgatgcttttgaactgtggtgttggagaagactcttgagagtcccttggactgcaaggagatccaaccagtccattctgaaggagatcagccctgggatttctttggaaggagtgatgctgaagctgaaactccagtactttggccacctcatgggaagagttgactcattggaaaatactctgatgctgggaaggattgggggcaggaggagaaggggaagacagagatgaaatggctggatggcatcactgactcgatggacatgagtttgagtgaactccgggagttagtgatggacagggagacctgcgtgctgtgattcatggggtcacaaagagttggacaggaccgagtgactgaactgaactgaatgcccctgacaggactagataacgtactcctttgtcattactgtaggtctaagatatgaaatgtttaccattggagacacactaaactattcttataaaagactgggggaggaaatcagtgacttacattccctacagcaataagagaataagtctgatggctatttcaccaagttctcagttttagggcactgacttggattgctttacatcatg

SEQ ID NO: 7 - DNA, 254 bp, Bovine
atgaggagacaggttggacggctggcagtcatcattgtgggcgccattgtcagtaagctccttgaagttcttcacagaccacccaggtccctcttgggatcaccagcaaatactcagctccctgattgggcccaggagtccattgatttgctgccaccgtcctgtcctctgagaatactcataactgtcccaagactcggctccctgaatgccatcccagctcctgacccttgcatgttttctcttttcactag

SEQ ID NO: 8 - protein, 84 aa, Bovine
MRRQVGRLAVIIVGAIVSKLLEVLHRPPRSLLGSPANTQLPDWAQESIDLLPPSCPLRILITVPRLGSLNAIPAPDPCMFSLCH

SEQ ID NO: 9 - DNA, 1532 bp, Bovine
gacgcgggaggagaaaggctttacaattttggaacttccatggacgacccatgaggagacaggttggacggctggcagtcatcattgtgggcgccattgtcagtaagctccttgaagttcttcacagaccacccaggtccctcttgggatcaccagcaaatactcagctccctgattgggcccaggagtccattgatttgctgccaccgtcctgtcctctgagaatactcataactgtcccaagactcggctccctgaatgccatcccagctcctgacccttgcatgttttctctttgtcactagattcagaatggaagccacaaaatgaaacatgctatttactctgactttgccgctctggttttcctcctgcctggtgcttgtttttcatttgactttttccaaaatgagaagagaaaagggctctagatttctcgaagagatcggacccttctaccttgagaatgcctttttgtaatgtctacattttaaaactatcaaactgaacagggcagattttaagtaggcttacagattaggatcattgcaatacattgcagctaaatatcagaactagatgatgtcagctctcatcggaatgaccaactaagaactgaagtgatgtaggaactacttgggtccctaacattcaagaggtaatattccatgagcagatgtcagttaacaatgttagccaacgagacatatcagttcctgagcatatgttggatgttagctgatcagtactgatattaatcatactttggacagtagacagtctgtaaacacccagttgacaccaaagaaaataactgatttgattttctgctattgctyttgaagccctgaccgtatctcttgtcatacttgagataagttcactgattatgttccatctacaccctgtatatactctgtggttgggatttactgctttctattctaattatttgtttaccagttccatactagcctgtaagactgtgctggggttgtgctttattactgtttgccaggcctacaatggcataagtaggagcttgggagtaaaaaacgatttggatctaaatcttggctcgagaagtttgtgtatcatcatgggcaaattaactactctggacctcataatatgaatctgaaaacaggaatttttttaattgaagtatcattgattcaccatgctgtgttagattcaggtacacagcagagggatccagttatatatattagttttcggattcttcttccctagtgaacagtttcctgtgctatacagtagatccttgttggtcacctgctctgtatatagtcgtgtgtagatgttaatcccaaactcctaatttatctctcatccccccttcccgctttggtaaccataggtttgttttctatgtctgtggatctgtttctgtaaaacccttgtttttaggtattttaatacctacctggaagagttcattagcactgtgatgtaatgtactatattataaaggttctttggttgttaacagaaaaaaaaaaaagaaaaaa

SEQ ID NO: 10 - DNA, 30 bp, Bovine
ctggaagttcttcacagaccacccaggtcc

SEQ ID NO: 11 - DNA, 30 bp, Bovine
cttgaggttcttcacagaccacccaggtcc

SEQ ID NO: 12 - DNA, 30 bp, Bovine
cttgaagttcttcatagaccacccaggtcc

SEQ ID NO: 13 - DNA, 30 bp, Bovine
ctggaggttcttcacagaccacccaggtcc

SEQ ID NO: 14 - DNA, 30 bp, Bovine
ctggaggttcttcatagaccccccaggtcc

SEQ ID NO: 15 - DNA, 30 bp, Bovine
cttgaagttcttcatagaccccccaggtcc

SEQ ID NO: 16 - DNA, 30 bp, Bovine
cttgaagttcttcacagaccacccagatca

SEQ ID NO: 17 - DNA, 30 bp, Bovine
cttgaagttcttcacagaccccccagatca

SEQ ID NO: 18 - DNA, 30 bp, Bovine
ctggaggttcttcatagaccccccagatca

SEQ ID NO: 19 - DNA, 30 bp, Bovine
ctggaggttctgcatagaccccccagatca

SEQ ID NO: 20 - DNA, 30 bp, Bovine
attgaagttcttcacagaccacccaggtcc

SEQ ID NO: 21 - DNA, 30 bp, Bovine
cttgaagctcttcacagaccacccaggtcc

SEQ ID NO: 22 - DNA, 30 bp, Bovine
cttgatgttcttcacagaccacccaggtcc

SEQ ID NO: 23 - DNA, 30 bp, Bovine
cttgaagctcttcacagaccacccaaatcc

SEQ ID NO: 24 - DNA, 30 bp, Bovine
cttgaagttcttcacagaccacccaaatcc

SEQ ID NO: 25 - DNA, 30 bp, Bovine
cttgaagttcttcacagaccaggcaggtcc

SEQ ID NO: 26 - DNA, 30 bp, Bovine
cttgaagagcttcacagaccacccaggtcc

SEQ ID NO: 27 - DNA, 30 bp, Bovine
cttgatgttcttcacagaccaggcaggtcc

SEQ ID NO: 28 - DNA, 30 bp, Bovine
cttgaagttcttaaaagaccacccaggtcc

SEQ ID NO: 29 - DNA, 30 bp, Bovine
cttgaagttcttcacagaccacccaggtac

SEQ ID NO: 30 - DNA, 21 bp, Artificial Sequence
  Synthetic
ttggaacttccatggacgacc

SEQ ID NO: 31 - DNA, 21 bp, Artificial Sequence
  Synthetic
atttgctggtgatcccaagag

SEQ ID NO: 32 - DNA, 29 bp, Artificial Sequence
  Synthetic
aatgggtggtctgtgaagaaccctgtctc

SEQ ID NO: 33 - DNA, 29 bp, Artificial Sequence
  Synthetic
aagttcttcacagaccacccacctgtctc

SEQ ID NO: 34 - DNA, 29 bp, Artificial Sequence
  Synthetic
aacaatcagggagctgagtatcctgtctc

SEQ ID NO: 35 - DNA, 29 bp, Artificial Sequence
  Synthetic
aaatactcagctccctgattgcctgtctc

SEQ ID NO: 36 - DNA, 25 bp, Artificial Sequence
  Synthetic
aaatctgtgtggatagccttatcag

SEQ ID NO: 37 - DNA, 23 bp, Artificial Sequence
  Synthetic
cctggtgacaaagagaacatacg

SEQ ID NO: 38 - DNA, 26 bp, Artificial Sequence
  Synthetic
ccaggcatgttacttatgaataactt

SEQ ID NO: 39 - DNA, 19 bp, Artificial Sequence
  Synthetic
agggagctgaagcttggaa

SEQ ID NO: 40 - DNA, 20 bp, Artificial Sequence
  Synthetic
tcctccctggagaagagcta

SEQ ID NO: 41 - DNA, 20 bp, Artificial Sequence
  Synthetic
agtacttgcgctcaggagga

SEQ ID NO: 42 - DNA, 51 bp, Artificial Sequence
  Synthetic
gactagttctagatcgcgagcggccgctttttttttttttttttttttttt

SEQ ID NO: 43 - DNA, 42 bp, Artificial Sequence
  Synthetic
aagcagtggtaacaacgcagagtacgaattcgtcgacgcggg

SEQ ID NO: 44 - DNA, 25 bp, Artificial Sequence
  Synthetic
aagcagtggtaacaacgcagagtac

SEQ ID NO: 45 - DNA, 23 bp, Artificial Sequence
  Synthetic
gactagttctagatcgcgagcgg

SEQ ID NO: 46 - DNA, 21 bp, Artificial Sequence
  Synthetic
cgccatggatgatgatattac

SEQ ID NO: 47 - DNA, 17 bp, Artificial Sequence
  Synthetic
aagccggccttgcacat

SEQ ID NO: 48 - DNA, 21 bp, Artificial Sequence
  Synthetic
ttggaacttccatggacgacc

SEQ ID NO: 49 - DNA, 22 bp, Artificial Sequence
  Synthetic
tcattttgtggcttccattctg

SEQ ID NO: 50 - DNA, 9 bp, Bovine
auuuuaaaa

SEQ ID NO: 51 - DNA, 10 bp, Bovine
uauuuuaaua

SEQ ID NO: 52 - DNA, 18 bp, Bovine
gctttacagtgagtactg

SEQ ID NO: 53 - DNA, 18 bp, Bovine
gtactctcagattttgga

SEQ ID NO: 54 - DNA, 18 bp, Bovine
ccttgaaggtagggataa

SEQ ID NO: 55 - DNA, 18 bp, Bovine
gtttttccagttcttcac

(19) United States
(12) Patent Application Publication
(10) Pub. No.: US 2009/0263406 A1
(43) Pub. Date: Oct. 22, 2009

(54) JY-1 Regulation Of Granulosa Cell Function And Early Embryonic Development In Cattle

(75) Inventors: Jianbo Yao, Morgantown, WV (US); George W. Smith, Onondaga, MI (US); Anilkumar Bettegowda, Houston, TX (US); Paul M. Coussens, Floshing, MI (US); James J. Ireland, East Lansing, MI (US)

Correspondence Address: Peter G. Carroll;Medlen & Carroll, LLP, 101 Howard Street, Suite 350, San Francisco, CA, 94105, US

(73) Assignee: Michigan State University

(21) Appl. No.: 12/258,800

(22) Filed: Oct. 27, 2008

Related U.S. Application Data
(60) Provisional application No. 61/001,006, filed on Oct. 30, 2007.

(51) Int. Cl. A61K 39/00; C07K 14/00; C07K 16/00; C07H 21/02; A61K 31/7088; A61K 38/16; A61P 15/00

(52) U.S. Cl. 424/184.1; 530/350; 530/387.9; 536/23.1; 514/44 A; 514/12

(57) ABSTRACT

The present invention provides compositions and methods for regulating fertility in mammals. In general, the invention relates to a novel protein produced by oocytes named JY-1, and nucleic acids encoding the JY-1 protein, for controlling folliculogenesis and early embryonic development, particularly in monoovulatory species. In particular, the present invention provides nucleic acid and amino acid sequences encoding JY-1, vectors for the expression of JY-1, host cells expressing JY-1, RNAi probes for reducing levels of JY-1 message, and antibodies to JY-1. Specifically, developing and mature oocytes express JY-1 in vivo, while granulosa cells treated in vitro with recombinant JY-1 (rJY-1) protein reduced cell proliferation while increasing progesterone synthesis and estradiol production. Further, reducing JY-1 protein in developing embryos in vitro using inhibitory siRNA constructs corresponded with arrested blastocyte maturation.

This application claims priority to U.S. Provisional Application Ser. No. 61/001,006, filed Oct. 30, 2006, now abandoned, herein incorporated by reference in its entirety.

### FIELD OF THE INVENTION

The present invention provides compositions and methods for regulating fertility in mammals. In general, the invention relates to a novel protein produced by oocytes named JY-1, and nucleic acids encoding the JY-1 protein, for controlling folliculogenesis and early embryonic development, particularly in monoovulatory species. In particular, the present invention provides nucleic acid and amino acid sequences encoding JY-1, vectors for the expression of JY-1, host cells expressing JY-1, RNAi probes for reducing levels of JY-1 message, and antibodies to JY-1. Specifically, developing and mature oocytes express JY-1 in vivo, while granulosa cells treated in vitro with recombinant JY-1 (rJY-1) protein reduced cell proliferation while increasing progesterone synthesis and estradiol production. Further, reducing JY-1 protein in developing embryos in vitro using inhibitory siRNA constructs corresponded with arrested blastocyte maturation.

### BACKGROUND OF THE INVENTION

Stem cell culture and nuclear transplant technologies have rapidly gained importance in the past few years. Indeed, these technologies have spawned an entire industry. (See, e.g., Lanza et al., “Cloning Noah's Ark,” Scientific American, 2000; Shamblott et al., (1998) Proc. Natl. Acad. Sci. USA 95(23):13726-31; Thomson et al. (1998) Science 282(5391):1145-47; Cibelli et al., (2002) “Science 295:819; all of which are herein incorporated by reference).

Despite the resources currently being devoted to these technologies, the efficiencies of nuclear transfer, especially somatic cell nuclear transfer and establishment of stem cell cultures remain low. A recipient oocyte is an absolute requirement for nuclear transfer yet the efficiency is very low. Merely between 1 and 4% of reconstructed embryos derived from somatic cell nuclear transfer develop to adulthood (Wilmut and Patterson, Oncol. Res. 13 (6-10):303-7 (2003)); herein incorporated by reference. Astonishingly, it is currently believed that fewer than 11 human embryonic stem cell lines are available for research. (See, e.g., “NIH says stem cell supply is low” Boston Globe, May 9, 2003, p. A3; herein incorporated by reference).

These statistics indicate that a substantial amount of research is still needed in order to better understand factors that regulate development in oocytes and early embryos on a molecular level.

### SUMMARY OF THE INVENTION

The present invention provides compositions and methods for regulating fertility in mammals. In general, the invention relates to a novel protein produced by oocytes named JY-1, and nucleic acids encoding the JY-1 protein, for controlling folliculogenesis and early embryonic development, particularly in monoovulatory species. In particular, the present invention provides nucleic acid and amino acid sequences encoding JY-1, vectors for the expression of JY-1, host cells expressing JY-1, RNAi probes for reducing levels of JY-1 message, and antibodies to JY-1. Specifically, developing and mature oocytes express JY-1 in vivo, while granulosa cells treated in vitro with recombinant JY-1 (rJY-1) protein reduced cell proliferation while increasing progesterone synthesis and estradiol production. Further, reducing JY-1 protein in developing embryos in vitro using inhibitory siRNA constructs corresponded with arrested blastocyte maturation.

The present invention provides an isolated nucleic acid sequence that hybridizes to a nucleic acid sequence selected from the group consisting of SEQ ID NOs: 1-7, 9, and complementary sequences thereof, under conditions of at least medium stringency. In one embodiment, said nucleic acid sequence hybridizes to a nucleic acid sequence selected from the group consisting of a human chromosome 11 nucleic acid sequence, chimpanzee chromosome 11 nucleic acid sequence, dog chromosome 21 nucleic acid sequence, mouse chromosome 7 nucleic acid sequence, and rat chromosome 1 nucleic acid sequence. It is not intended that the nucleic acid sequence be limited to a particular genus or species. Indeed, it is contemplated that the isolated nucleic acid sequence is derived from a nucleic acid sequence selected from the group comprising, but not limited to members of the genera bovine, human, ovine, equine, porcine and caprine. In one embodiment, said nucleic acid sequence is selected from the group consisting of SEQ ID NOs: 1-7, 9, and complementary sequences thereof. In one embodiment, said nucleic acid sequence is SEQ ID NO:7. In one embodiment, said nucleic acid sequence encodes a polypeptide selected from the group consisting of SEQ ID NO:8 and a variant of SEQ ID NO:8. In one embodiment, said variant of SEQ ID NO:8 comprises at least one sequence as set forth in SEQ ID NOs: 10-29. In one embodiment, the invention provides a vector comprising the nucleic acid sequence that hybridizes to a nucleic acid sequence selected from the group consisting of SEQ ID NOs: 1-7, 9, and complementary sequences thereof, under conditions of at least medium stringency. In one embodiment, the invention provides a host cell comprising a vector comprising the nucleic acid sequence that hybridizes to a nucleic acid sequence selected from the group consisting of SEQ ID NOs: 1-7, 9, and complementary sequences thereof, under conditions of at least medium stringency. In one embodiment, said host cell is located in an organism. In one embodiment, said organism is a monoovulatory species. It is not intended that the organism be limited to a particular genus or species. Indeed, it is contemplated that the organism is selected from the group comprising, but not limited to members of monoovulatory and polyovulatory species. Indeed, it is contemplated that the organism is selected from the group comprising, but not limited to human, non-human primate, cattle, bison, buffalo, water buffalo, African buffalo, zebu, banteng, gaur, yak, antelope, gazelle, reindeer, moose, giraffe, bactrian camel, dromedary camel, camelid, deer, elk, caribou, swine, goat, sheep, big-horn sheep, horse, pony, donkey, zebra, mule, llama, alpaca, vicuña, guanaco, and hybrids thereof. In other embodiments, it is contemplated that the organism is selected from the group comprising, but not limited to mouse, rat, chicken, and fish, such as rainbow trout, and zebrafish.

The present invention provides an isolated nucleic acid sequence selected from the group consisting of SEQ ID NOs: 1, 2, 3, 4, 5, 6, 7, 9, variants thereof, and complementary sequences thereof at least 75% identical to SEQ ID NOs: 1, 2, 3, 4, 5, 6, 7, or 9. In one embodiment, said nucleic acid sequence is at least 76%, 85%, 90%, 99%, or 100% identical to SEQ ID NOs:1, 2, 3, 4, 5, 6, 7, or 9.

The present invention provides an isolated polypeptide encoded by a nucleic acid selected from the group consisting of SEQ ID NOs: 1, 2, 3, 4, 5, 6, 7, 9, and variants thereof at least 75% identical to SEQ ID NOs:1, 2, 3, 4, 5, 6, 7, or 9. In one embodiment, said nucleic acid sequence is at least 76%, 85%, 90%, 99%, or 100% identical to SEQ ID NOs:1, 2, 3, 4, 5, 6, 7, or 9.

The present invention provides a purified antibody that binds specifically to an isolated polypeptide encoded by a nucleic acid selected from the group consisting of SEQ ID NOs: 1, 2, 3, 4, 5, 6, 7, 9, and variants thereof at least 75% identical to SEQ ID NOs:1, 2, 3, 4, 5, 6, 7, or 9. In one embodiment, said nucleic acid sequence is at least 76%, 85%, 90%, 99%, or 100% identical to SEQ ID NOs:1, 2, 3, 4, 5, 6, 7, or 9.

The present invention provides a composition comprising a nucleic acid sequence that inhibits the binding of at least a portion of a nucleic acid selected from the group consisting of SEQ ID NOs: 1, 2, 3, 4, 5, 6, 7, 9 to it's complementary sequence. Indeed, a variety of inhibitory sequences are contemplated as being useful in the present inventions. Indeed, it is contemplated that said nucleic acid sequence is selected from but not limited to the group consisting of SEQ ID NO:32-38.

The present invention provides a polynucleotide sequence comprising at least fifteen nucleotides capable of hybridizing under stringent conditions to an isolated nucleic acid sequence selected from the group consisting of SEQ ID NOs: 1-7 and 9.

The present invention provides a method comprising: a) providing, i) an inhibitory JY-1 RNA; and ii) a host target cell expressing a sense JY-1 nucleotide sequence; and b) introducing said inhibitory JY-1 RNA into said host target cell under conditions such that said sense JY-1 nucleotide sequence expression levels are reduced.

In one embodiment, said inhibitory JY-1 RNA is an siRNA. In one embodiment, said siRNA is selected from the group consisting of SEQ ID NOs:32-35. In one embodiment, said inhibitory JY-1 RNA further comprises an RNA expression vector.

The present invention provides a method for screening compounds for altering JY-1 protein activity, comprising: a) providing, i) a host cell expressing a JY-1 protein, and ii) a test compound capable of altering JY-1 protein activity; b) treating said host cell with said test compound; and c) detecting altered JY-1 protein activity. In one embodiment, said detecting further comprises identifying a test compound bound to a JY-1 protein.

The present invention provides a method for screening compounds for the ability to alter JY-1 expression, comprising: a) providing, i) a host cell comprising a vector construct, wherein said vector construct is capable of expressing a JY-1 nucleotide sequence and ii) a test compound capable of altering said nucleotide expression; b) treating said host cell with said test compound; and c) detecting the effect of said test compound on said JY-1 expression. In one embodiment, said nucleotide sequence is selected from the group consisting of SEQ ID NOs:2-6, wherein said sequence is operably linked to a reporter gene.

The present invention provides a vaccine comprising a JY-1 polypeptide selected from the group consisting of SEQ ID NOs: 1, 7, 9, and a variant thereof.

The present invention provides a method for altering in vivo fertility, comprising: a) providing: i) a female subject, wherein said subject comprises a cell selected from the group consisting of an oocyte and an embryonic cell; and ii) a composition comprising a polypeptide, wherein said polypeptide is selected from the group consisting of SEQ ID NO:08 and a variant of SEQ ID NO:08, and b) injecting said female subject with said composition under conditions that alter in vivo fertility. In one embodiment, said composition increases fertility. In one embodiment, said alteration of fertility comprises enhancing oocyte development. In one embodiment, said alteration of fertility is decreasing fertility. In one embodiment, said method further comprises, providing, an agent for altering fertility and injecting said agent into said subject. In one embodiment, said agent is selected from the group consisting of gonadotropin hormone, chorionic gonadotropin hormone, luteinizing hormone, growth hormone, follicle stimulating hormone, steroidogenic acute regulatory protein (StAR), and Cocaine- and Amphetamine-Regulated Transcript (CART). In one embodiment, said subject is selected from the group consisting of monoovulatory species. In one embodiment, said subject is selected from the group consisting of human, non-human primate, cattle, bison, buffalo, water buffalo, African buffalo, zebu, banteng, gaur, yak, antelope, gazelle, reindeer, moose, giraffe, bactrian camel, dromedary camel, camelid, deer, elk, caribou, swine, goat, sheep, big-horn sheep, horse, pony, donkey, zebra, mule, llama, alpaca, vicufia, guanaco, and hybrids thereof. In one embodiment, said subject is selected from the group consisting of Bovidae, Homimidae, Salmonidae, and Cyprimidae. In one embodiment, said subject is selected from the group consisting of mouse, chicken, rainbow trout, zebrafish, human, bovine, equine, porcine, ovine, elk, and bison. In one embodiment, said subject is selected from the group consisting of human, ovine, equine, porcine and caprine. In one embodiment, said variant is selected from the group consisting of human, ovine, equine, porcine and caprine.

The present invention provides a method for increasing in vitro fertility, comprising: a) providing: i) an oocyte cell; ii) oocyte culture medium; iii) a composition comprising a polypeptide, wherein said polypeptide is selected from the group consisting of SEQ ID NO:08 and a variant of SEQ ID NO:08, and; b) adding said oocyte cell and said composition to said culture medium; and c) culturing said oocyte cell under conditions that said oocyte cell increases in fertility. In one embodiment, said culturing generates an oocyte expressing a maturation marker. In one embodiment, said culturing generates an oocyte decreasing a developmental marker.

The present invention provides a method for alteration of in vitro fertility, comprising: a) providing: i) a target cell, wherein said target cell is an embryonic cell; and ii) a composition comprising a polypeptide, wherein said polypeptide is selected from the group consisting of SEQ ID NO:08, a variants of SEQ ID NO:08; and b) culturing said embryonic cell with said composition under conditions that alter embryonic cell development. In one embodiment, said culturing generates an embryonic cell in a blastocoel stage. In one embodiment, the method further comprises providing a host. In one embodiment, the method further comprises transplanting said cultured embryonic cell into said host. In one embodiment, said host is selected from the group consisting of human, ovine, equine, porcine and caprine. In one embodiment, said composition further comprises at least one growth factor. Indeed, a variety of growth factors are contemplated as being useful to the present inventions. In one embodiment, said growth factor is contemplated but not limited to stem cell factor, Fms-like Tyrosine Kinase-3, and thrombopoietin.

The present invention provides kit comprising a rJY-1 protein and instructions for use.

The present invention provides kit comprising a JY-1 inhibitory RNA and instructions for use.

The present invention provides kit comprising a JY-1 siRNA cocktail and instructions for use.

The present invention provides kit comprising a JY-1 antibody and instructions for use.

### DESCRIPTION OF THE FIGURES

FIG. 1 shows an exemplary genomic sequence of JY-1 (SEQ ID NO:1) and component sequences (SEQ ID NOs:2-6).

FIG. 2 shows an exemplary open reading frame for JY-1 (SEQ ID NO:7).

FIG. 3 shows an exemplary amino acid sequence of JY-1 (SEQ ID NO:8).

FIG. 4 shows exemplary flanking sequences of the open reading frame for JY-1 (underlined) (SEQ ID NO:9).

FIG. 5 shows an exemplary characterization of JY-1 mRNA and protein. (A) Northern analysis of JY-1 mRNA in multiple bovine tissues and (B) adult germinal vesicle stage oocytes (GVO). (C-E) Western blot detection of JY-1 protein in bovine oocytes using antisera generated against recombinant JY-1 protein (a mature form of JY-1 lacking signal peptide; rJY-1). (C) Representative Western blot demonstrating detection of immunoreactive JY-1 protein of approximately 11,000 Mr in lysates of 150 germinal vesicle oocytes (GVO) and immunoreactivity of rJY-1 (approximately 6,700 Mr). (D) Duplicate blot to (C) incubated with JY-1 anti-serum pre-absorbed with excess rJY-1 protein which blocked binding of antibody to JY-1 protein in GVO and to rJY-1. (E) Representative Western blots demonstrating tissue specificity of JY-1 immunoreactivity. Note absence of immunoreactive JY-1 in samples of bull serum, granulosa cells, liver and adrenal gland. (F) Duplicate blot to (E) demonstrating absence of JY-1 immunoreactivity when blot was incubated with preimmune serum.

FIG. 6 shows exemplary micrographs demonstrating intraovarian localization of JY-1 protein. (A) Representative bright field micrograph of a preantral follicle stained with hematoxylin and eosin. (B) The corresponding dark field micrograph of (A) demonstrating oocyte-specific localization of JY-1 mRNA. (C-E) Immunohistochemical localization of JY-1 protein to the oocytes of (C) growing follicles, (D) primordial follicles and (E) primary follicles. Arrows (D and E) indicate a primordial and a primary follicle. (F) Localization of JY-1 protein to the oocyte of an antral follicle. (G-H) Adjacent section to that depicted in (F) incubated with (G) pre-immune IgG or with (H) JY-1 antibody pre-absorbed with excess antigen. A and B, Magnification, ×400. C, Magnification, ×200. D and E, Magnification, ×1000. F-H, Magnification, ×100.

FIG. 7 shows exemplary micrographs demonstrating intraovarian localization of JY-1 mRNA. (A) Representative bright field micrograph of an antral follicle hybridized with 35S-labeled antisense JY-1 cRNA and stained with hematoxylin and eosin. (B) Representative dark-field micrograph of the same section depicted in (A). Note oocyte specific localization of JY-1 mRNA. A and B, Magnification, ×400. GC, granulosa cell layer; OO, oocyte.

FIG. 8 shows exemplary experimental effects of recombinant JY-1 protein (rJY-1) on granulosa cell numbers and estradiol (E) and progesterone (P) production. (A) Effect of rJY-1 on total granulosa cell numbers for FSH treated cells. Note decrease in cell numbers with increasing concentration of rJY-1 (P<0.05). (B) Effect of rJY-1 on FSH stimulated E production by bovine granulosa cells. Concentrations of E were decreased in response to 0.1 ng/ml rJY-1 and the response was maximal at 0.5 ng/ml rJY-1 (P<0.05). (C) Effect of rJY-1 on P production by FSH treated bovine granulosa cells. Note dose dependent increase in P in response to increasing concentrations of rJY-1 (P<0.01). Concentrations of E and P were normalized to 30,000 cells. Data are depicted as mean±SEM.

FIG. 9 shows an exemplary quantification of JY-1 mRNA abundance during oocyte maturation and early embryogenesis and effect of JY-1 knockdown on blastocyst development. (A) Relative abundance of polyadenylated JY-1 mRNA transcripts during meiotic maturation through embryonic genome activation [germinal vesicle (GV) and metaphase (MII) stage (oocytes), pronucleus (PN), 2-cell (2C), 4-cell (4C), 8-cell (8C), and 16-cell (16C) stage (embryos)]. Data are normalized relative to abundance of exogenous control (GFP) RNA and shown as mean±SEM. (B) Effect of JY-1 siRNA microinjection on abundance of polyadenylated JY-1 mRNA in samples of 2-cell embryos. Denuded MII oocytes were either microinjected with sham water or JY-1 siRNA cocktail followed by parthenogenetic activation. Data were normalized relative to abundance of endogenous control 18S rRNA and shown as mean±SEM. (C, D) Effect of JY-1 knockdown on development of parthenogenetic (C) or IVF (D) embryos to the blastocyst stage. Denuded MII oocytes or presumptive zygotes were microinjected with sham water, JY-1 siRNA cocktail, negative (−) control (Ctrl) siRNA or served as uninjected controls. Average rates of blastocyst development were calculated and data are shown as mean±SEM. Time points without a common superscript are significantly different, P<0.05.

FIG. 10 shows an exemplary Quantification of JY-1 mRNA abundance during oocyte maturation and early embryogenesis and effect of JY-1 knockdown on blastocyst development. (A) Dynamic changes in relative abundance of polyadenylated JY-1 mRNA transcripts during meiotic maturation and prior to embryonic genome activation [germinal vesicle (GV) and metaphase (MII) stage (oocytes), pronucleus (PN), 2-cell (2C), 4-cell (4C), 8-cell (8C), and 16-cell (16C) stage (embryos)]. (B) Gradual decline in abundance of total JY-1 transcripts in same samples (n=5 each) as depicted in (A). Oligo dT(18) primers were used to reverse transcribe polyadenylated transcripts, whereas random hexamers were used to reverse transcribe total transcripts. Data are normalized relative to abundance of exogenous control (GFP) RNA and shown as mean±SEM. (C) Effect of JY-1 knockdown on parthenogenetic blastocyst development. Denuded MII oocytes were subjected to one of the following microinjection treatments: (1) uninjected control, (2) sham water injection (3) JY-1 siRNA cocktail and (4) negative (−) control siRNA (n=25-30 embryos per treatment). Microinjected oocytes were parthenogenetically activated and rates of blastocyst development were recorded on day 7 and the experiment replicated 4×. (D) Effect of JY-1 knockdown on development of IVF embryos to the blastocyst stage. Presumptive one cell IVF embryos were subjected to microinjection treatments as in (C). Microinjected embryos were cultured until day 7 and rates of blastocyst development recorded. The experiment was replicated 5×. JY-1 siRNA injection dramatically decreased the proportion of parthenogenetic and IVF embryos developing to the blastocyst stage. Average rates of blastocyst development were calculated and data are shown as mean±SEM. Time points without a common superscript are significantly different, P<0.05.

FIG. 11 shows an exemplary genomic organization and characterization of putative cis-elements in the 5′-flanking region of the bovine JY-1 gene. (A) The JY-1 gene has three exons separated by two introns. Exonic and intronic sequences at the exon-intron junctions are shown in upper-case and lower-case letters, respectively. The donor (gt) and acceptor (ag) splice sites corresponding to the first and last two bases of the intron, and are underlined. The donor (gt) and acceptor (ag) splice sites are in agreement with consensus sequences. (B) Putative cis-elements in the 5′ flanking region of the bovine JY-1 gene.

FIG. 12 shows exemplary detection of JY-1 like sequences in the genome of multiple species and structure of JY-1 gene. A) Genomic Southern blot hybridized with ³²P labeled 450 bp JY-1 cDNA (probe) spanning the open reading frame (ORF), 5′ UTR and a portion of the 3′UTR. Note strong hybridization to a bovine genomic DNA fragment and weaker hybridization to sheep, pig and human genomic DNA. (B) Gene structure of bovine JY-1. The JY-1 gene has 3 exons (E1, E2, E3) separated by two introns. The start (ATG) and stop (TAG) codons of the open reading frame are indicated within the exons. (C) Characterization of JY-1-like sequences in the genome of additional species. Genomic DNA databases at NCBI for human, chimpanzee, dog, mouse, rat, chicken, zebrafish and drosophila were searched with the nucleotide sequence of the 1.5 kb bovine JY-1 cDNA. JY-1-like sequences corresponding to exon 3 were identified on human chromosome (chr)-11, chimpanzee chr-11, dog chr-21, mouse chr-7 and rat chr-1 (syntenic chromosomes to human chr-11). JY-1 like sequences were not identified in public genomic DNA databases for chicken, zebrafish and drosophila.

FIG. 13 shows an exemplary detection of JY-1 like sequences, structure of JY-1 gene and cloning of a putative human mRNA ortholog of bovine JY-1. A) Genomic Southern blot hybridized with ³²P labeled 450 bp JY-1 cDNA (probe) spanning the open reading frame (ORF), 5′ UTR and a portion of the 3′UTR. Southern blot was prepared with genomic DNA from cow (lane 1), sheep (lane 2), pig (lane 3), human (lane 4), mouse (lane 5), chicken (lane 6), rainbow trout (lane 7) and zebrafish (lane 8). Note strong hybridization to a bovine genomic DNA fragment. Weaker hybridization signals were also detected in sheep, pig and human genomic DNA. (B) Gene structure of bovine JY-1. The JY-1 gene has 3 exons (25, 92 and 1400 bp in length) separated by two introns (12.8 and 1.5 kb in length). The three exons in the gene are marked as E1, E2 and E3 respectively. (C) Detection of putative human mRNA ortholog of bovine JY-1 in adult human ovary and H9 human embryonic stem (ES) cell RNA using RT-PCR. Amplification of the housekeeping gene beta-actin (β-actin) with PCR primers spanning two introns was used as a positive control to verify cDNA synthesis and to confirm absence of genomic DNA contamination [1, 3=cDNA synthesis in the presence of reverse transcriptase; 2, 4=negative control; cDNA synthesis in absence of reverse transcriptase].

FIG. 14 shows an exemplary characterization of JY-1 like sequences in the human genome. (A) Alignment of bovine JY-1 cDNA with DNA fragments on the long arm of human chromosome 11 (11q14). The human genomic DNA database at NCBI was searched with the nucleotide sequence encoding for the longest bovine JY-1 cDNA (1.5 kb). Identical JY-1-like sequence was identified at two loci on human chromosome 11 with region of similarity corresponding to 187 bp of the protein coding region and 850 bp in the 3′UTR. (B) Alignment of bovine JY-1 cDNA with a single human EST sequence derived from a Hembase [human erythroid precursor cell (adult stem cell)] cDNA library. The region of sequence similarity in the Hembase EST is 187 bp and corresponds to human chromosome 11 (11q14) (http://hembase.niddk.nih.gov). (C) Alignment of Hembase human EST with DNA fragments present on human chromosome 11. The human Hembase EST aligned to human chromosome 11 (100% identity) at two locations identical to the loci where the sequences similar to bovine JY-1 are present.

FIG. 15 shows an exemplary characterization of the number and size of JY-1 mRNA transcripts. (A) Northern analysis of JY-1 mRNA in multiple bovine tissues and (B) adult germinal vesicle stage oocytes (GVO). Three predominant JY-1 transcripts were detected in RNA from fetal ovaries and GVO. (C) Characterization of JY-1 cDNA clones derived from oocyte and fetal ovary cDNA libraries. Note two shorter clones of 450 bp and 350 bp derived from the oocyte library and two larger clones of 1.5 and 1 kb from fetal ovary library representing four distinct transcripts with identical open reading frame (ORF) but differing lengths of the 3′-untranslated region (3′UTR). UA rich cytoplasmic polyadenylation elements (CPE) are present in the 3′UTR of the two largest transcripts.

FIG. 16 shows an exemplary RT-PCR analysis of JY-1 mRNA transcripts in multiple bovine tissues. Samples of fetal ovary tissue (an enriched source of oocytes), fetal testis, spleen, heart, muscle, lung, adult testes, uterus, thymus, kidney, liver, adrenal gland, hypothalamus, brain cortex, gut, pituitary, bone marrow (sternum and leg) and leukocytes were subjected to RNA isolation, DNAse digestion and cDNA synthesis as described previously [Matzuk, et al., Science 2002; 296:2178-2180, herein incorporated by reference]. Amplification of JY-1 cDNA was performed using specific primers (F: 5′-TTGGAACTTCCATGGACGACC-3′ and R: 5′-TCATTTTGTGGCTTCCATTCTG-3′) and standard PCR procedures. Amplification of a 360 bp cDNA encoding for bovine RPL-19 was used as a positive control to confirm that cDNA synthesis was successful.

FIG. 17 shows an exemplary quantitative real-time RT-PCR analysis of total versus polyadenylated JY-1 mRNA transcripts within in vitro derived early bovine embryos. (A) Relative abundance of polyadenylated JY-1 transcripts in samples collected from 16-cell (16C) through blastocyst stages (n=5 samples of 10 embryos per sample at 16C, morula and blastocyst stage). (B) Relative abundance of total JY-1 transcripts in same samples (n=5 each) as depicted in (A). 250 femtograms of polyadenylated GFP mRNA were added to each sample prior to RNA extraction. Oligo dT(18) primers were used to reverse transcribe polyadenylated transcripts, whereas random hexamers were used to reverse transcribe total transcripts. cDNA corresponding to 1/10th of an oocyte or embryo was used for real time analysis. Data were normalized relative to abundance of exogenous control (GFP) RNA and are shown as mean±SEM. Time points with a common superscript are not significantly different.

FIG. 18 shows an exemplary quantitative real-time RT-PCR analysis of JY-1 mRNA within in vitro derived embryos cultured with or without the RNA polymerase II inhibitor α-amanitin. (A) Experimental model utilized for α-amanitin treatment. Bovine embryos were cultured with α-amanitin (25 μg/ml) between 24 to 33 h or between 33 to 44 h post fertilization (during first and second bovine embryonic cell cycles). The 2-cell and 4-cell embryos from untreated control and α-amanitin treatment groups were embryos from untreated control and α-amanitin treatment groups were collected at 33 and 44 h post fertilization respectively. (B) Effects of α-amanitin on blastocyst development. From each in vitro fertilization (IVF) run, a proportion of α-amanitin treated and control embryos were cultured until day 7 and percentage of embryos reaching the blastocyst stage recorded. None of the embryos treated with α-amanitin developed to the blastocyst stage. (C) Relative abundance of polyadenylated JY-1 mRNA transcripts in control and α-amanitin treated 2-cell and 4-cell embryos [n=4 samples of 10 embryos each for 2-cell (2C) and 4-cell (4C) stage embryos; untreated control embryos [α-amanitin (−)]; α-amanitin treated embryos [α amanitin (+)]. Samples were spiked with 250 femtograms of GFP RNA prior to RNA extraction. Oligo dT(18) primers were used in reverse transcription. cDNA corresponding to 1/0th of an embryo was used for real time analysis. Data were normalized relative to abundance of exogenous control (GFP) RNA and shown as mean±SEM. Means with common superscripts were not different. Results demonstrate an abundance of JY-1 mRNA transcripts detected in 2-cell and 4-cell embryos was not affected by α-amanitin treatment suggesting that such transcripts were not synthesized in the embryos but rather are of oocyte origin.

FIG. 19 shows an exemplary validation of oocyte/embryo microinjection procedure. Metaphase II stage bovine oocytes were denuded of cumulus cells by vortexing in 0.1% hyaluronidase enzyme in Hamster embryo culture medium. Each denuded oocyte was secured to the holding pipet with brief negative suction. The ICSI microinjection pipet was loaded with Texas-red Dextran dye by aspiration. During the microinjection procedure, the oocyte cytoplasm was briefly aspirated into the microinjection pipet with negative suction pressure to ensure the breakage of cytoplasmic membrane before dye injection. Microinjected oocytes were parthenogenetically activated by 4 min incubation in 5 μM ionomycin followed by 4 h incubation in 2 mM 6-dimethylaminopurine (6-DMAP) and then cultured for 7 days. Representative bright field micrographs taken at (A) 48 h and (C) day 7 of embryonic development. The corresponding dark field micrographs are presented in Panel B and D. The success rate of microinjection procedure was calculated based on total number of embryos retaining fluorescent dye at 48 hr after activation over total number of oocytes microinjected. Results demonstrate that the success rate of microinjection was greater than 90%.

FIG. 20 shows an exemplary effect of negative control siRNA injection on blastocyst quality. Denuded MII bovine oocytes were either uninjected or microinjected with universal negative control siRNA #1. The oocytes were parthenogenetically activated by 4 min incubation in 5 μM ionomycin followed by 4 h incubation in 2 mM 6-dimethylaminopurine (6-DMAP) and cultured for seven days. Blastocyst cell number (an index of blastocyst quality [Eppig, 2002 et al., Proc Natl Acad Sci U.S.A. 99:2890-2894; Li, et al., 2000 Biol Reprod 63: 839-845; herein incorporated by reference] was determined by the blue fluorescent Hoechst nuclear staining (n=9-15 embryos per treatment). For cell counts, blastocysts were incubated in PBS solution containing Hoechst dye (5 μg/ml) and BSA (3 mg/ml) for 10 minutes. Embryos were mounted on glass slides in drops of glycerol and sealed with a cover slip. The cell numbers for each blastocyst were counted under an inverted Nikon fluorescent microscope fitted with a UV filter. Average cell numbers for blastocysts derived from uninjected control and negative control siRNA microinjection are shown as mean±SEM. Means with common superscripts are not different. Results demonstrate that quality of blastocysts derived from negative control siRNA injection (as determined by day 7 cell numbers) is not different from uninjected control blastocysts.

FIG. 21 shows an exemplary validation of JY-1 siRNA species for efficacy of JY-1 mRNA knockdown in samples of 2-cell embryos. Denuded metaphase II bovine oocytes were either microinjected with water or JY-1 siRNA cocktail (25 mM; species 1+2). Microinjected oocytes were parthenogenetically activated by 4 min incubation in 5 μM ionomycin followed by 4 h incubation in 2 mM 6-dimethylaminopurine (6-DMAP) and 2-cell embryo samples were collected at 33 h after injection and parthenogenetic activation (n=4 samples of 5 embryos each) for RNA isolation and cDNA synthesis. Relative abundance of JY-1 transcripts was quantified by real-time PCR analysis. Oligo dT(18) primers were used to synthesize cDNA. Data were normalized relative to abundance of endogenous control 18S rRNA by the following formula: 2-CT [Eppig, Reproduction 2001; 122:829-838, herein incorporated by reference]. Means without a common superscript are significantly different, P<0.05. Results demonstrate that microinjection of the JY-1 siRNA cocktail reduces JY-1 mRNA abundance by approximately 90% in 2-cell embryos within 33 h after activation.

FIG. 22 shows an exemplary validation of JY-1 siRNA species for efficacy of JY-1 mRNA knockdown in samples of 4-cell embryos. Denuded metaphase II bovine oocytes were either microinjected with water or individual JY-1 siRNA species at two different doses (25 μM and 50 μM). Microinjected oocytes were parthenogenetically activated by 4 min incubation in 5 μM ionomycin followed by 4 h incubation in 2 mM 6-dimethylaminopurine (6-DMAP) and 4-cell embryo samples were collected at 41-43 h after activation (n=4 samples of 5 embryos each) for RNA isolation and quantification of JY-1 mRNA abundance by real-time PCR. (A) Relative abundance of JY-1 transcripts in samples subjected to sham (water) or JY-1 siRNA species-1 injection. (B) Relative abundance of JY-1 transcripts in samples subjected to sham (water) or JY-1 siRNA species-2 injection. Oligo dT(18) primers were used to synthesize cDNA. Data were normalized relative to abundance of endogenous control 18S rRNA by the following formula: 2-CT [Eppig, Reproduction 2001 122:829-838, herein incorporated by reference]. Results demonstrate that both siRNA species 1 and 2 (at 25 μM concentration) reduce JY-1 mRNA abundance by approximately 90% in 4-cell embryos within 41-43 h after injection and parthenogenetic activation.

FIG. 23 shows an exemplary effect of JY-1 siRNA microinjection on JY-1 protein abundance in 8-16 cell embryos. Denuded MII bovine oocytes were either uninjected or microinjected with JY-1 siRNA species (I+2) cocktail. The oocytes were parthenogenetically activated by 4 min incubation in 5 μM ionomycin followed by 4 h incubation in 2 mM 6-dimethylaminopurine (6-DMAP) and 8-16 cell embryo samples were collected at 72 h after activation. Protein lysates of 185 eight-to-sixteen cell embryos per treatment per lane were used in Western blot analysis. Note absence of immunoreactive JY-1 protein (11000 Mr) in samples of embryos microinjected with JY-1 siRNA. Membrane was stripped and probed for actin. No significant difference in actin protein abundance was observed between the two treatments.

FIG. 24 shows an exemplary validation of JY-1 and universal negative control siRNA species for specificity of target mRNA recognition. Denuded metaphase II bovine oocytes were subjected to three different microinjection treatments 1) Sham water, 2) JY-1 siRNA species (1+2) cocktail and 3) universal negative control siRNA-1 (Ambion). Microinjected oocytes were parthenogenetically activated by 4 min incubation in 5 μM ionomycin followed by 4 h incubation in 2 mM 6-dimethylaminopurine (6-DMAP) and 4-cell embryo samples were collected at 41-43 h after activation (n=4 samples of 5 embryos each) for RNA isolation and cDNA synthesis. Samples were spiked with 250 femtograms of GFP RNA prior to RNA extraction. Oligo dT(18) primers were used in reverse transcription. Relative abundance of (A) JY-1, (B) 18S rRNA, (C) beta-actin, (D) glyceraldehyde-3-phosphate dehydrogenase (GAPDH), (E) ribosomal protein L-19 (RPL-19), (F) cyclophilin-A and (G) ribosomal protein L-15 (RPL-15) transcripts in samples of 4-cell embryos were quantified by real-time PCR. Data are normalized relative to abundance of endogenous control 18S rRNA by the following formula: 2-CT [Eppig, Reproduction 2001; 122:829-838, herein incorporated by reference]. The relative abundance of 18S rRNA was determined by normalizing to exogenous control GFP RNA. The negative control siRNA species did not reduce abundance of any of the RNA transcripts examined. Results demonstrate specificity of JY-1 siRNA species in reducing JY-1 mRNA but none of the other RNA transcripts examined.

FIG. 25 shows an exemplary effect of JY-1 mRNA knockdown on bovine early embryonic development. Presumptive one cell bovine embryos derived from in vitro fertilization were subjected to one of the following treatments: 1) JY-1 cocktail siRNA (25 μM), 2) negative control siRNA-1 (25 μM), 3) sham water injection or 4) uninjected controls. Injected embryos were cultured for seven days and rates of blastocyst development recorded. Representative micrographs of day 7 embryos derived from (A) uninjected controls, (B) Sham water injection (C) negative control siRNA injection and (D) JY-1 siRNA cocktail injection are shown. The day 7 blastocysts are indicated by pointed arrows. Results demonstrate JY-1 siRNA injection reduces the development of IVF embryos to the blastocyst stage.

FIG. 26 shows an exemplary comparison of JY-1 mRNA knockdown, between species 1 and 2, on bovine early embryonic development. Presumptive one cell bovine embryos derived from in vitro fertilization were subjected to one of the following treatments: 1) uninjected controls, 2) sham water, 3) JY-1 siRNA-1 and 4) JY-1 siRNA-2. Injected embryos were cultured for seven days and rates of blastocyst development recorded. Results demonstrated that both JY-1 siRNA 1 and 2 injection reduced the development of IVF embryos to the blastocyst stage

FIG. 27 shows an exemplary amplification of JY-1 from a variety of species as described herein.

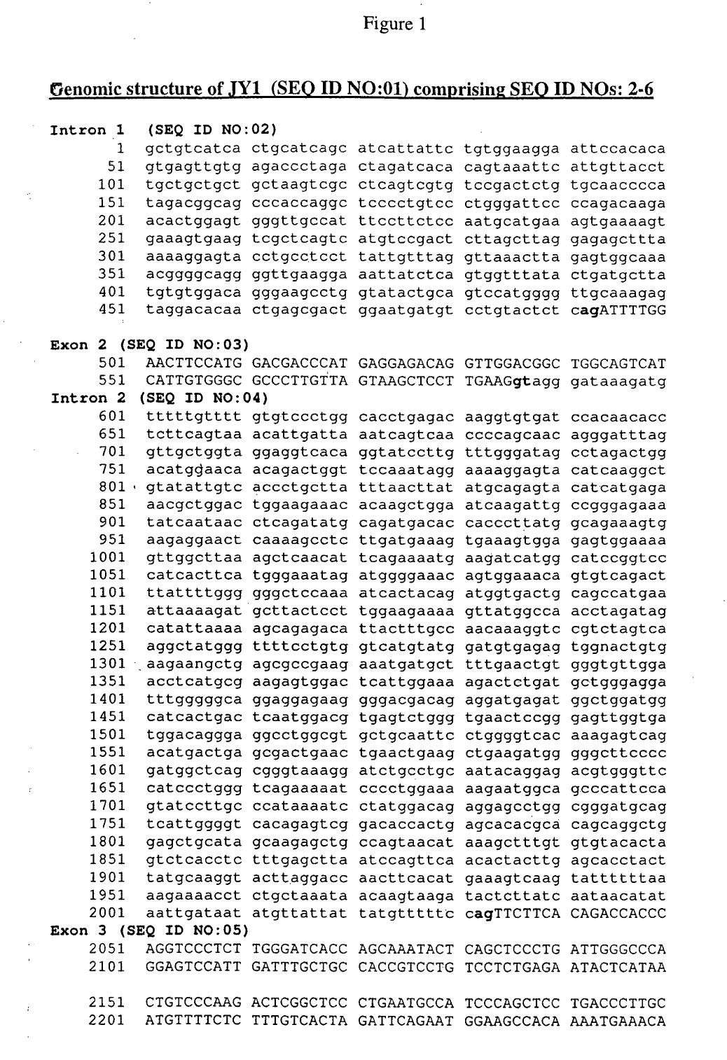

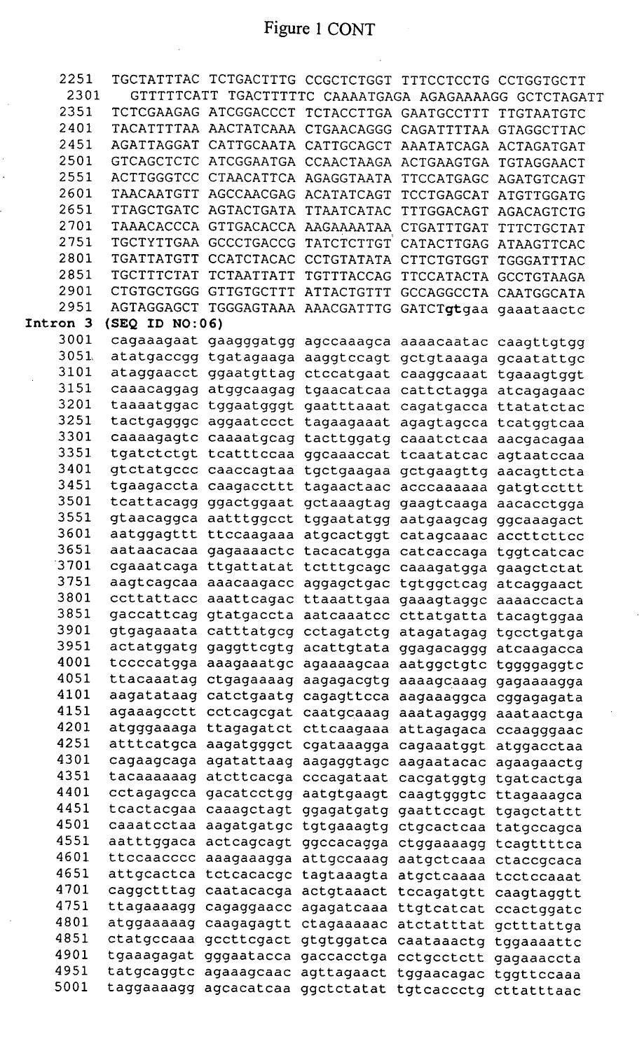

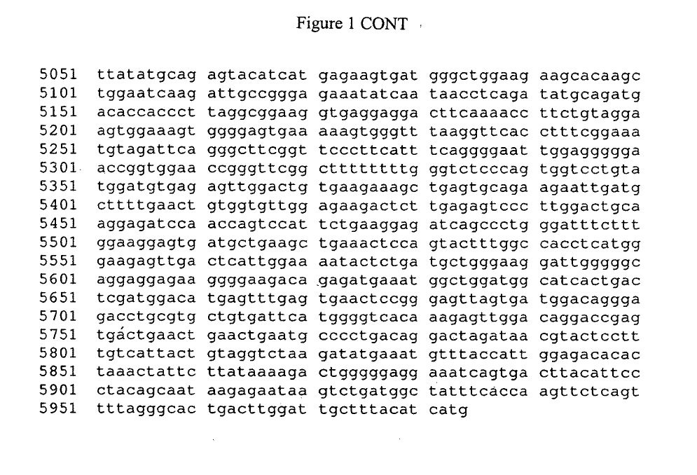

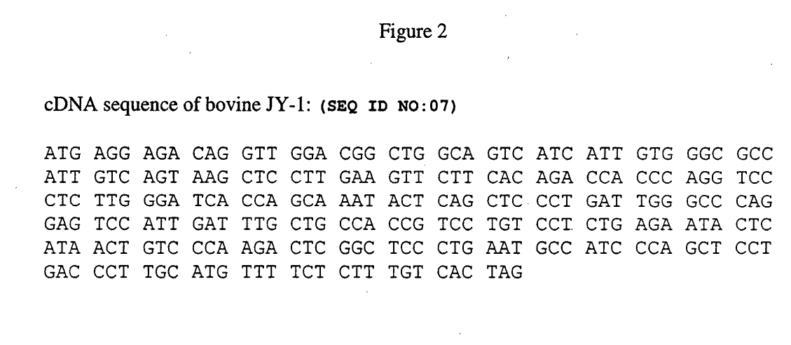

### DEFINITIONS

To facilitate understanding of the invention, a number of terms are defined below.

As used herein, the term “JY-1” when used in reference to a protein or nucleic acid refers to a protein or nucleic acid encoding a protein that is preferentially expressed in oocytes. The term JY-1 encompasses both proteins that are identical to wild-type JY-1 and those that are derived from wild type JY-1 (e.g., variants of JY-1 or chimeric genes constructed with portions of JY-1 coding regions). In some embodiments, the “JY-1” is the wild type nucleic acid (SEQ ID NOs: 1, 7 and 9) or amino acid (SEQ ID NO:8) sequence. In other embodiments, the “JY-1” is a variant or mutant (e.g., including, but not limited to, the nucleic acid sequences comprising nucleic acid sequences described by SEQ ID NOS: 10-29 and proteins comprising the amino acid sequences encoded by SEQ ID NOS: 10-29). In some embodiments, the “JY-1” refers to both the sense and the antisense nucleic acid sequences.

The term “gene” refers to a nucleic acid (e.g., DNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In particular, the term “JY-1 gene” refers to the full-length JY-1 nucleotide sequence (e.g., contained in SEQ ID NO: 1). However, it is also intended that the term encompass fragments of the JY-1 sequence, mutants, as well as other domains within the full-length JY-1 nucleotide sequence, for example, SEQ ID NO:7. Furthermore, the terms “JY-1 nucleotide sequence” or “JY-1 polynucleotide sequence” encompasses DNA, cDNA, and RNA (e.g., mRNA) sequences.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” “polymorphism,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term RNAi probe refers to RNA that mediates RNAi interference. This term includes probes that comprise double-stranded RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the 21-23 nt RNA or internally (at one or more nucleotides of the RNA). Nucleotides in the RNA molecules of the present invention can also comprise non-standard nucleotides, including non-naturally occurring nucleotides or deoxyribonucleotides. Collectively, all such altered RNAs are referred to as analogs or analogs of naturally-occurring RNA.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, the terms “complementary” or “complementarity” are used in reference to polynucleotides (i.e., a sequence of nucleotides) related by the base-pairing rules. For example, for the sequence 5′-“A-G-T-3′,” is complementary to the sequence 3′-“T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “competes for binding” is used in reference to a first polypeptide with an activity which binds to the same substrate as does a second polypeptide with an activity, where the second polypeptide is a variant of the first polypeptide or a related or dissimilar polypeptide. The efficiency (e.g., kinetics or thermodynamics) of binding by the first polypeptide may be the same as or greater than or less than the efficiency substrate binding by the second polypeptide. For example, the equilibrium binding constant (K_{D}) for binding to the substrate may be different for the two polypeptides. The term “Kₘ” as used herein refers to the Michaelis-Menton constant for an enzyme and is defined as the concentration of the specific substrate at which a given enzyme yields one-half its maximum velocity in an enzyme catalyzed reaction.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985], herein incorporated by reference). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed. The present invention is not limited to the hybridization of probes of about 500 nucleotides in length. The present invention contemplates the use of probes between approximately 10 nucleotides up to several thousand (e.g., at least 5000) nucleotides in length.

One skilled in the relevant understands that stringency conditions may be altered for probes of other sizes (See e.g., Anderson and Young, Quantitative Filter Hybridization, in Nucleic Acid Hybridization [1985] and Sambrook et al., Molecular Cloning: A Laboratory Manual, Cold Spring Harbor Press, NY [1989], all of which are herein incorporated by reference).

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970), herein incorporated by reference], by the search for similarity method of Pearson and Lipman [Pearson and Lipman, Proc. Natl. Acad. Sci. (U.S.A.) 85:2444 (1988), herein incorporated by reference], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. Alignments may also be conducted with publicly available programs such as BLAST, Gapped-BLAST and PSI-BLAST. See, e.g., Altschul, S. F., Gish, W., Miller, W., Myers, E. W. & Lipman, D. J. “Basic local alignment search tool.” J. Mol. Biol. 215:403-410 (1990); and Altschul, S. F., Madden, T. L., Schäffer, A. A., Zhang, J., Zhang, Z., Miller, W. & Lipman, D. J. “Gapped BLAST and PSI-BLAST: a new generation of protein database search programs.” Nucleic Acids Res. 25:3389-3402 (1997); herein incorporated by reference. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention (e.g., JY-1).

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions (claimed in the present invention) with its various ligands and/or substrates.

The term “polymorphic locus” is a locus present in a population that shows variation between members of the population (i.e., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

As used herein, the term “genetic variation information” or “genetic variant information” refers to the presence or absence of one or more variant nucleic acid sequences (e.g., polymorphism or mutations) in a given allele of a particular gene (e.g., the JY-1 gene).

As used herein, the term “detection assay” refers to an assay for detecting the presence of absence of variant nucleic acid sequences (e.g., polymorphism or mutations) in a given allele of a particular gene (e.g., the JY-1 gene). Examples of suitable detection assays include, but are not limited to, those described below.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to a nucleic acid sequence or structure to be detected or characterized. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule that is comprised of segments of DNA joined together by means of molecular biological techniques.

The term “sample” is used in its broadest sense. In one sense it can refer to a plant cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention. The term “sample” is used in its broadest sense. In one sense it can refer to a biopolymeric material. In another sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. These examples are not to be construed as limiting the sample types applicable to the present invention.

The term “antisense” refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex which is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA; many antisense RNAs block the expression of a target gene by interfering with the processing, transport and/or translation of its primary transcript, for example mRNA. The complementarity of an antisense RNA may be with any part of the specific gene transcript, i.e., at the 5′ non-coding sequence, 3′ non-coding sequence, introns, or the coding sequence. In addition, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein, or of preventing the function of a target RNA.

The term “siRNAs” refers to short interfering RNAs. In some embodiments, siRNAs comprise a duplex, or double-stranded region, where each strand of the double stranded region is about 18 to about 25 nucleotides long; the double stranded region can be as short as 16, and as long as 29, base pairs long, where the length is determined by the antisense strand. Often siRNAs contain from about two to four unpaired nucleotides at the 3′ end of each strand. siRNAs appear to function as key intermediaries in triggering RNA interference in invertebrates and in vertebrates, and in triggering sequence-specific RNA degradation during posttranscriptional gene silencing in plants. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. One strand of the double stranded region need not be the exact length of the opposite strand; thus, one strand may have at least one fewer nucleotides than the opposite complementary strand, resulting in a “bubble” or at least one unmatched base in the opposite strand. One strand of the double stranded region need not be exactly complementary to the opposite strand; thus, the strand, preferably the sense strand, may have at least one mismatched base-pair.

siRNAs may also contain additional sequences; non-limiting examples of such sequences include linking sequences, or loops, which connect the two strands of the duplex region. This form of siRNAs may be referred to “si-like RNA,” “short hairpin siRNA,” where the short refers to the duplex region of the siRNA, or “hairpin siRNA.” Additional non-limiting examples of additional sequences present in siRNAs include stem and other folded structures. The additional sequences may or may not have known functions; non-limiting examples of such functions include increasing stability of an siRNA molecule, or providing a cellular destination signal.

The term “target RNA molecule” refers to an RNA molecule to which at least one strand of the short double-stranded region of an siRNA is complementary. Typically, when such complementarity is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

The term “ds siRNA” refers to a siRNA molecule which comprises two separate unlinked strands of RNA which form a duplex structure, such that the siRNA molecule comprises two RNA polynucleotides.

The term “hairpin siRNA” refers to a siRNA molecule which comprises at least one duplex region where the strands of the duplex are connected or contiguous at one or both ends, such that the siRNA molecule comprises a single RNA polynucleotide. The antisense sequence, or sequence which is complementary to a target RNA, comprises at least a part of the at least one double stranded region.

The term “enhancing the function” when used in reference to an siRNA molecule means that the effectiveness of an siRNA molecule in silencing gene expression is increased. Such enhancements include but are not limited to increased rates of formation of an siRNA molecule, decreased susceptibility to degradation, and increased transport throughout the cell. An increased rate of formation might result from a transcript which possesses sequences which enhance folding or the formation of a duplex strand.

The term “RNA interference” or “RNAi” refers to the silencing or decreasing expression, or inhibition of expression, of gene expression by siRNAs. It is the process of sequence-specific, post-transcriptional gene silencing in animals and plants, initiated by siRNA that is homologous in its duplex region to the sequence of the silenced gene or that is complementary in its duplex region to the transcriptional product of the silenced gene. The gene may be endogenous or exogenous to the organism, present integrated into a chromosome or present in a transfection vector which is not integrated into the genome. The expression of the silenced gene is either completely or partially inhibited.

The term “underexpression” refers to a relative decrease in production of a gene product in a transgenic organism or a cell that is below levels of production in an untreated or a normal or a wild-type or a non-transformed organism or cell.

The term “overexpression” refers to the production of a gene product in a transgenic organism or a cell that exceeds levels of production in an untreated or a normal or a wild-type or a non-transformed organism or cell.

The term “cosuppression” refers to the expression of a foreign gene which has substantial homology to an endogenous gene resulting in the suppression of expression of both the foreign and the endogenous gene.

As used herein, the term “altered level” or “altering level” refers to the production of gene product(s) in a treated organism in an amount or a proportion that differs from that of an untreated or a normal or a wild-type or a non-transformed organism or cell.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding JY-1 includes, by way of example, such nucleic acid in cells ordinarily expressing JY-1 where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein, a “portion of a chromosome” refers to a discrete section of the chromosome. Chromosomes are divided into sites or sections by cytogeneticists as follows: the short (relative to the centromere) arm of a chromosome is termed the “p” arm; the long arm is termed the “q” arm. Each arm is then divided into 2 regions termed region 1 and region 2 (region 1 is closest to the centromere). Each region is further divided into bands. The bands may be further divided into sub-bands. For example, the 11p15.5 portion of human chromosome 11 is the portion located on chromosome 11 (11) on the short arm (p) in the first region (1) in the 5th band (5) in sub-band 5 (0.5). A portion of a chromosome may be “altered;” for instance the entire portion may be absent due to a deletion or may be rearranged (e.g., inversions, translocations, expanded or contracted due to changes in repeat regions). In the case of a deletion, an attempt to hybridize (i.e., specifically bind) a probe homologous to a particular portion of a chromosome could result in a negative result (i.e., the probe could not bind to the sample containing genetic material suspected of containing the missing portion of the chromosome). Thus, hybridization of a probe homologous to a particular portion of a chromosome may be used to detect alterations in a portion of a chromosome.

The term “sequences associated with a chromosome” means preparations of chromosomes (e.g., spreads of metaphase chromosomes), nucleic acid extracted from a sample containing chromosomal DNA (e.g., preparations of genomic DNA); the RNA that is produced by transcription of genes located on a chromosome (e.g., hnRNA and mRNA), and cDNA copies of the RNA transcribed from the DNA located on a chromosome. Sequences associated with a chromosome may be detected by numerous techniques including probing of Southern and Northern blots and in situ hybridization to RNA, DNA, or metaphase chromosomes with probes containing sequences homologous to the nucleic acids in the above listed preparations.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets, which specify stop codons (i.e., TAA, TAG, TGA).

As used herein, the term “purified” or “to purify” refers to the removal of contaminants from a sample. For example, JY-1 antibodies are purified by removal of contaminating non-immunoglobulin proteins; they are also purified by the removal of immunoglobulin that does not bind JY-1. The removal of non-immunoglobulin proteins and/or the removal of immunoglobulins that do not bind JY-1 results in an increase in the percent of JY-1-reactive immunoglobulins in the sample. In another example, recombinant JY-1 polypeptides are expressed in bacterial host cells and the polypeptides are purified by the removal of host cell proteins; the percent of recombinant JY-1 polypeptides is thereby increased in the sample.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989] herein incorporated by reference).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 [1989]; herein incorporated by reference).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “transgene” as used herein refers to a foreign, heterologous, or autologous gene that is placed into an organism by introducing the gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene. The term “autologous gene” is intended to encompass variants (e.g., polymorphisms or mutants) of the naturally occurring gene. The term transgene thus encompasses the replacement of the naturally occurring gene with a variant form of the gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Example 10, for a protocol for performing Northern blot analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots). The amount of mRNA present in the band corresponding in size to the correctly spliced JY-1 transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes.

The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]; herein incorporated by reference), has been modified by several groups to optimize conditions for particular types of cells. The art is well aware of these numerous modifications.

A “composition comprising a given polynucleotide sequence” as used herein refers broadly to any composition containing the given polynucleotide sequence. The composition may comprise an aqueous solution. Compositions comprising polynucleotide sequences encoding JY-1 (e.g., SEQ ID NO:1) or fragments thereof may be employed as hybridization probes. In this case, the JY-1 encoding polynucleotide sequences are typically employed in an aqueous solution containing salts (e.g., NaCl), detergents (e.g., SDS), and other components (e.g., Denhardt's solution, dry milk, salmon sperm DNA, etc.).

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the term “response,” when used in reference to an assay, refers to the generation of a detectable signal (e.g., accumulation of reporter protein, increase in ion concentration, accumulation of a detectable chemical product).

As used herein, the term “reporter gene” refers to a gene encoding a protein that may be assayed. Examples of reporter genes include, but are not limited to, luciferase (See, e.g., deWet et al., Mol. Cell. Biol. 7:725 [1987] and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

As used herein, the term “follicle” refers to an area of the ovary comprising an oocyte at any stage of oocyte development. A follicle may refer to any one of, primordial follicle, a growing follicle, primary follicle, a secondary follicle, a Graafian follicle, an antral follicle and a corpus luterum. Specifically, a primary follicle comprises an immature (primary) oocyte and a single layer of follicular cells or a mature oocyte and a number of layers of follicular cells; a secondary follicle comprises a primary oocyte or secondary oocytes, numerous layers of granulosa cells and fluid filled vesicular cavities; a Graafian follicle comprises a secondary oocytes, wherein the secondary oocytes is arrested before second meiotic division, multiple layers of granulosa cells, and a single large fluid filled cavity called an antrum; and a Luteal Phase (follicle) comprising an empty follicle also called a“corpus luteum” wherein said corpus luteum secretes estradiol and progesterone.

As used herein, the term “IVM” or “in-vitro maturation” refers to a maturation of an immature oocyte in vitro attempting to duplicate the natural process that occurs within the follicle in vivo.

As used herein, the term “IVF” or “in-vitro fertilization” refers to fertilization of an oocytes with a sperm outside of an organism. IVF may also refer to a technique, whereby oocytes and spermatozoa are mixed in the laboratory to achieve fertilization.

As used herein, the term “IVC” or “in-vitro culture” refers to an incubation of fertilized oocytes (zygotes) in the laboratory through the process of cleavage, typically up to the blastocyst stage of development.

As used herein, the term “PIP” or “in-vitro production” refers to a combined process of in-vitro maturation, in-vitro fertilization and in-vitro culture whereby embryos are produced in the laboratory (i.e. IVP=IVM+IVF+IVC).

As used herein, the term “intracytoplasmic sperm injection” refers to a micromanipulation procedure whereby a single spermatozoon is inserted directly into the cytoplasm of the oocyte to achieve fertilization during IVF.

As used herein, the term “inner cell mass” or “trophoblast” refers to a part of the blastocyst that will give rise to the embryo proper, as opposed to the extra-embryonic membranes.

As used herein, the term “implantation” refers to a process whereby the blastocyst stage embryo burrows into the lining of the uterus, or endometrium, to establish a pregnancy.

As used herein, the term “infertility” refers to a state of being infertile, for example, not being able to conceive, either in vivo or in vitro, or not being able to support development in order to deliver, either naturally or by cesarean, a live child.

As used herein, the term “fertility” refers to a state of being fertile, for example, being able to conceive, either in vivo or in vitro, or being able to support development in order to deliver, either naturally or by cesarean, a live child.

As used herein, the term “instructions” as in “instructions for using said kit for said detecting JY-1” includes instructions for using the reagents contained in the kit for the detection of variant and wild type JY-1 polypeptides. In some embodiments, the instructions further comprise the statement of intended use required by the United States (U.S) Food and Drug Administration (FDA) in labeling in vitro diagnostic products. The FDA classifies in vitro diagnostics as medical devices and requires that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the Federal Food, Drug, and Cosmetic Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use. Where applicable, photographs or engineering drawings should be supplied; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; 8) Any additional information regarding the in vitro diagnostic product requested that is necessary for the FDA to make a substantial equivalency determination. Additional information is available at the Internet web page of the United States Food and Drug Administration.

### DETAILED DESCRIPTION OF THE INVENTION

The present invention provides compositions and methods for regulating fertility in mammals. In general, the invention relates to a novel protein produced by oocytes named JY-1, and nucleic acids encoding the JY-1 protein, for controlling folliculogenesis and early embryonic development, particularly in monoovulatory species. In particular, the present invention provides nucleic acid and amino acid sequences encoding JY-1, vectors for the expression of JY-1, host cells expressing JY-1, RNAi probes for reducing levels of JY-1 message, and antibodies to JY-1. Specifically, developing and mature oocytes express JY-1 in vivo, while granulosa cells treated in vitro with recombinant JY-1 (rJY-1) protein reduced cell proliferation while increasing progesterone synthesis and estradiol production. Further, reducing JY-1 protein in developing embryos in vitro using inhibitory siRNA constructs corresponded with arrested blastocyte maturation.

Accordingly, the present invention provides nucleic acid sequences, amino acid sequences, recombinant peptides, interfering RNA sequences, antibodies, probes, vectors, and other compositions relating to a novel protein expressed in bovine oocytes that was named JY-1.

JY-1 was discovered by the inventors using a PCR-based cDNA amplification procedure (SMART, BD Biosciences) for identifying highly expressed sequences in a bovine oocyte cDNA library constructed using RNA isolated from 200 bovine oocytes (metaphase II and germinal vesicle stages), see, Examples herein.

Initial analysis of 114 EST sequences from the bovine oocyte cDNA library (non-normalized) revealed novel information about oocyte-expressed genes. The 114 ESTs represented 45 unique genes. One EST sequence was selected by the inventors for further analysis (this sequence was represented by 14 fully sequenced clones with 2 different sizes; 455 bp and 355 bp) because it appeared to be novel and did not show significant homology to sequences of any known genes or ESTs deposited in Genbank. The name JY-1 was assigned to this gene. Identification of this novel JY-1 EST was significant because there were currently 4.9 million human, 3.7 million mouse and approximately 228,000 bovine EST sequences deposited in Genbank (TIGR release 7.0). JY-1 cDNA clones were present in the bovine oocyte library at a frequency of 12.2%. Thus, it appeared that JY-1 was a major component of the bovine oocyte transcriptome.

The inventors found that JY-1 protein was localized to oocytes of follicles at the primary through Graafian follicle stages, similar to the localization pattern observed for JY-1 mRNA.

Using the longest bovine JY-1 cDNA sequence (1.5 kb), a search was conducted for homologous sequences in a Genbank database. One significant match was identified in the nonredundant (nr) database as a human genomic fragment from chromosome 11. About 500 bp of sequence in the 3′UTR plus a portion of the coding region of the JY-1 cDNA shared approximately 80% similarity with this human genomic sequence. This level of similarity is common between bovine and human species of a homologous gene. By searching an additional Genbank database, which contained high throughput draft genomic sequences (htgs), a genomic sequence from mouse chromosome 7 (syntenic with human chromosome 11) was found that showed partial sequence similarity with the bovine JY-1 cDNA. The region of sequence similarity was limited to about 400 bp of sequence in the 3′UTR of the bovine cDNA.

Growth of an oocyte is accompanied by proliferation of adjacent granulosa cells to form the antral follicle from which a mature egg ovulates. The granulosa cells surround the oocyte within the follicle. During the follicular phase of the female cycle granulosa cells secrete estradiol, then in the luteal phase they secrete progesterone. Specialized granulosa cells, cumulus cells, a subset of granulosa cells, form an oocyte-cumulus complex.

Throughout growth, the oocyte remains arrested at prophase I of meiosis, which is characterized by a prominent nucleus (the germinal vesicle; GV). Disappearance, or “breakdown” or “G VBD” or “germinal vesicle breakdown” of the germinal vesicle (actually a prolonged late prophase stage of the first meiotic division) signals the primary oocyte's resumption of meiosis.

Oocyte growth is an essential component of oogenesis, since oocytes become capable of resuming meiosis when they reach about 80% of their final size in the mouse (Sorensen and Wassarman, 1976, Dev. Biol. 50:531-536, herein incorporated by reference; Wassarman et al., 1979, Adv. Exp. Med. Biol. 112:251-268, herein incorporated by reference). Meiosis normally resumes as a result of the preovulatory surge in luteinising hormone, and progresses to metaphase II (a process termed meiotic maturation), whereupon the oocyte is ovulated in most mammals, including the mouse, and re-arrests pending successful fertilisation.

Successful in-vitro fertilization, especially in monoovulatory species, requires “super ovulation” in order to provide more than one oocytes from an ovary for increasing IVF and successful embryonic implementation.

Currently, FSH (follicle stimulating hormone) is used to stimulate development of multiple follicles, GnRH-agonist (gonadotropin releasing hormone agonist) or a GnRH-antagonist (e.g. Ganirelix) is used to suppress an LH surge and ovulation until the follicles are mature while HCG (human chorionic gonadotropin) causes final maturation of the oocytes in the follicles.

A Luteinizing hormone (LH) surge is a surge in LH secretion during the late follicular phase of the female cycle, caused by positive feedback of rising estradiol levels on the pituitary. The surge acts as the trigger for the final stages of oocyte maturation and ovulation called luteinization. Gonadotrophin induces a “luteinization” of the granulosa cells, such that luteinizatuion refers to a change in the granulosa cells of the follicle, induced by the LH surge that causes their steroid hormone production to switch from estradiol to progesterone.

Ovulation triggers a Luteal phase or second half of the female cycle, during which the luteinized follicle or corpus luteum secretes progesterone and estradiol. The Luteal phase is also when implantation of the blastocyst into the endometrium lining the uterus occurs.

Currently, there are several factors that are limiting factors for increasing fertility in vivo and in vitro. For example, success of in vivo fertility is directly related to and limited by the number of mature eggs released upon ovulation.

Therefore, compositions and methods of the present invention have a variety of uses. For example, JY-1 protein or peptides derived from JY-1 can be used to immunize subjects to effect contraception. JY-1 proteins and peptides may also be used clinically to treat eggs and embryos utilized for in vitro maturation and fertilization procedures, and also to effect nuclear reprogramming in cloned embryos. RNAi probes developed from the JY-1 sequences of the present invention can be used to decrease expression of JY-1 protein in cells. Furthermore, it is expected that natural variation in the JY-1 gene will correlate with egg quality and other reproductive parameters.

### I. JY-1 Polynucleotides.

The present invention provides nucleic acids encoding JY-1 genes, homologs, variants (e.g., polymorphisms and mutants), including but not limited to, those described in SEQ ID NOs: 1, 7 and 9. In some embodiments, the present invention provides polynucleotide sequences that are capable of hybridizing to SEQ ID NOs: 1, 7 and 9 under conditions of low to high stringency as long as the polynucleotide sequence capable of hybridizing encodes a protein that retains a biological activity of the naturally occurring JY-1. In some embodiments, the protein that retains a biological activity of naturally occurring JY-1 is 70% homologous to wild-type JY-1, preferably 80% homologous to wild-type JY-1, more preferably 90% homologous to wild-type JY-1, and most preferably 95% homologous to wild-type JY-1. In preferred embodiments, hybridization conditions are based on the melting temperature (Tm) of the nucleic acid binding complex and confer a defined “stringency” as explained above (See e.g., Wahl, et al., Meth. Enzymol., 152:399 407 [1987], incorporated herein by reference).

In other embodiments of the present invention, additional alleles of JY-1 are provided. In preferred embodiments, alleles result from a polymorphism or mutation (i.e., a change in the nucleic acid sequence) and generally produce altered mRNAs or polypeptides whose structure or function may or may not be altered. Any given gene may have none, one or many allelic forms. Common mutational changes that give rise to alleles are generally ascribed to deletions, additions or substitutions of nucleic acids. Each of these types of changes may occur alone, or in combination with the others, and at the rate of one or more times in a given sequence. Examples of the alleles of the present invention include those encoded by SEQ ID NOs: 1 and 7 (wild type).

In still other embodiments of the present invention, the nucleotide sequences of the present invention may be engineered in order to alter an JY-1 coding sequence for a variety of reasons, including but not limited to, alterations which modify the cloning, processing and/or expression of the gene product. For example, mutations may be introduced using techniques that are well known in the art (e.g., site-directed mutagenesis to insert new restriction sites, to alter glycosylation patterns, to change codon preference, etc.).

In some embodiments of the present invention, the polynucleotide sequence of JY-1 may be extended utilizing the nucleotide sequence (e.g., SEQ ID NO: 1) in various methods known in the art to detect upstream sequences such as promoters and regulatory elements. For example, it is contemplated that restriction-site polymerase chain reaction (PCR) will find use in the present invention. This is a direct method that uses universal primers to retrieve unknown sequence adjacent to a known locus (Gobinda et al., PCR Methods Applic., 2:318-22 [1993], herein incorporated by reference). First, genomic DNA is amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

In another embodiment, inverse PCR can be used to amplify or extend sequences using divergent primers based on a known region (Triglia et al., Nucleic Acids Res., 16:8186 [1988], herein incorporated by reference). The primers may be designed using Oligo 4.0 (National Biosciences Inc, Plymouth Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template. In still other embodiments, walking PCR is utilized. Walking PCR is a method for targeted gene walking that permits retrieval of unknown sequence (Parker et al., Nucleic Acids Res., 19:3055-60 [1991], herein incorporated by reference). The PROMOTERFINDER kit (Clontech) uses PCR, nested primers and special libraries to “walk in” genomic DNA. This process avoids the need to screen libraries and is useful in finding intronlexon junctions.

Preferred libraries for screening for full-length cDNAs include mammalian libraries that have been size-selected to include larger cDNAs. Also, random primed libraries are preferred, in that they will contain more sequences that contain the 5′ and upstream gene regions. A randomly primed library may be particularly useful in case where an oligo d(T) library does not yield full-length cDNA. Genomic mammalian libraries are useful for obtaining introns and extending 5′ sequence.

In other embodiments of the present invention, variants of the disclosed JY-1 sequences are provided. In preferred embodiments, variants result from polymorphisms or mutations (i.e., a change in the nucleic acid sequence) and generally produce altered mRNAs or polypeptides whose structure or function may or may not be altered. Any given gene may have none, one, or many variant forms. Common mutational changes that give rise to variants are generally ascribed to deletions, additions or substitutions of nucleic acids. Each of these types of changes may occur alone, or in combination with the others, and at the rate of one or more times in a given sequence.

It is contemplated that it is possible to modify the structure of a peptide having a function (e.g., JY-1 function) for such purposes as altering the biological activity. Such modified peptides are considered functional equivalents of peptides having an activity of JY-1 as defined herein. A modified peptide can be produced in which the nucleotide sequence encoding the polypeptide has been altered, such as by substitution, deletion, or addition. In particularly preferred embodiments, these modifications do not significantly reduce the biological activity of the modified JY-1. In other words, construct “X” can be evaluated in order to determine whether it is a member of the genus of modified or variant JY-1's of the present invention as defined functionally, rather than structurally.

Moreover, as described above, variant forms of JY-1 are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present invention provide variants of JY-1 disclosed herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (e.g., Stryer ed., Biochemistry, pg. 17-21, 2nd ed, WH Freeman and Co., 1981, herein incorporated by reference). Whether a change in the amino acid sequence of a peptide results in a functional polypeptide can be readily determined by assessing the ability of the variant peptide to function in a fashion similar to the wild-type protein. Peptides having more than one replacement can readily be tested in the same manner.

More rarely, a variant includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

As described in more detail below, variants may be produced by methods such as directed evolution or other techniques for producing combinatorial libraries of variants, described in more detail below. In still other embodiments of the present invention, the nucleotide sequences of the present invention may be engineered in order to alter a JY-1 coding sequence including, but not limited to, alterations that modify the cloning, processing, localization, secretion, and/or expression of the gene product. For example, mutations may be introduced using techniques that are well known in the art (e.g., site-directed mutagenesis to insert new restriction sites, alter glycosylation patterns, or change codon preference, etc.).

Table 1 presents silent mutations in a subregion of the JY-1 coding region. Table 2 presents conservative and nonconservative mutations in a subregion of the JY-1 coding region. It is recognized that this subregion represents a portion of the larger JY-1 coding region. As explained above, sequences containing such mutations, along with deletion and addition mutations, are variant sequences according to the present invention. These examples are intended to be non-limiting and provide a subset of sequences that exemplify the sequences encompassed by the present invention.

TABLE 1

| | Sequences with Silent Changes |
|---|---|
| SEQ ID NO: | (Changes in bold) |
| 10 | CTG GAA GTT CTT CAC AGA CCA CCC AGG TCC |
| 11 | CTT GAG GTT CTT CAC AGA CCA CCC AGG TCC |
| 12 | CTT GAA GTT CTT CAT AGA CCA CCC AGG TCC |
| 13 | CTG GAG GTT CTT CAC AGA CCA CCC AGG TCC |
| 14 | CTG GAG GTT CTT CAT AGA CCC CCC AGG TCC |
| 15 | CTT GAA GTT CTT CAT AGA CCC CCC AGG TCC |
| 16 | CTT GAA GTT CTT CAC AGA CCA CCC AGA TCA |
| 17 | CTT GAA GTT CTT CAC AGA CCC CCC AGA TCA |
| 18 | CTG GAG GTT CTT CAT AGA CCC CCC AGA TCA |
| 19 | CTG GAG GTT CTG CAT AGA CCC CCC AGA TCA |

TABLE 2

| | Sequences with Amino Acid Changes |
|---|---|
| SEQ ID NO: | (Changes in bold) |
| 20 | ATT GAA GTT CTT CAC AGA CCA CCC AGG TCC |
| 21 | CTT GAA GCT CTT CAC AGA CCA CCC AGG TCC |
| 22 | CTT GAT GTT CTT CAC AGA CCA CCC AGG TCC |
| 23 | CTT GAA GCT CTT CAC AGA CCA CCC AAA TCC |
| 24 | CTT GAA GTT CTT CAC AGA CCA CCC AAA TCC |
| 25 | CTT GAA GTT CTT CAC AGA CCA GGC AGG TCC |
| 26 | CTT GAA GAG CTT CAC AGA CCA CCC AGG TCC |
| 27 | CTT GAT GTT CTT CAC AGA CCA GGC AGG TCC |
| 28 | CTT GAA GTT CTT AAA AGA CCA CCC AGG TCC |
| 29 | CTT GAA GTT CTT CAC AGA CCA CCC AGG TAC |

### II. JY-1 Polypeptides.

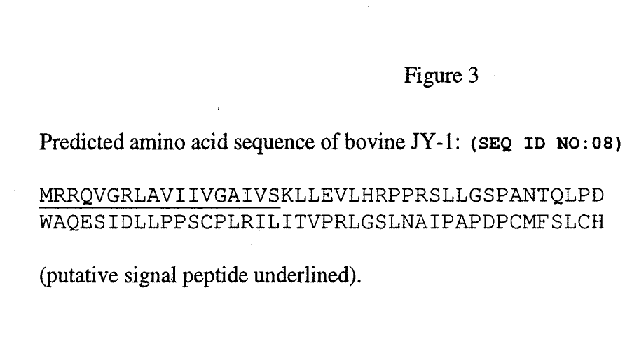

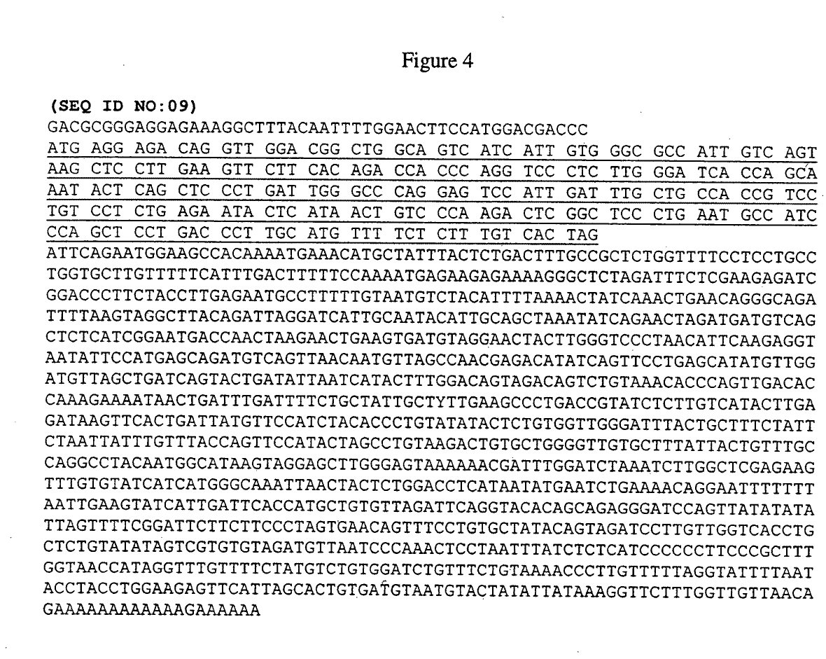

In other embodiments, the present invention provides JY-1 polynucleotide sequences that encode JY-1 polypeptide sequences. The JY-1 polypeptide sequence (SEQ ID NO: 8) is provided in FIG. 3. Other embodiments of the present invention provide fragments, fusion proteins or functional equivalents of these JY-1 proteins. In some embodiments, the present invention provides truncation mutants of JY-1. In still other embodiments of the present invention, nucleic acid sequences corresponding to JY-1 variants, homologs, and mutants may be used to generate recombinant DNA molecules that direct the expression of the JY-1 variants, homologs, and mutants in appropriate host cells. In some embodiments of the present invention, the polypeptide may be a naturally purified product, in other embodiments it may be a product of chemical synthetic procedures, and in still other embodiments it may be produced by recombinant techniques using a prokaryotic or eukaryotic host (e.g., by bacterial, yeast, higher plant, insect and mammalian cells in culture). In some embodiments, depending upon the host employed in a recombinant production procedure, the polypeptide of the present invention may be glycosylated or may be non-glycosylated. In other embodiments, the polypeptides of the invention may also include an initial methionine amino acid residue.

In one embodiment of the present invention, due to the inherent degeneracy of the genetic code, DNA sequences other than the polynucleotide sequences of SEQ ID NOs: 1, 7 and 9 that encode substantially the same or a functionally equivalent amino acid sequence, may be used to clone and express JY-1. In general, such polynucleotide sequences hybridize to SEQ ID NOs: 1, 7 and 9 under conditions of high to medium stringency as described above. As will be understood by those of skill in the art, it may be advantageous to produce JY-1-encoding nucleotide sequences possessing non-naturally occurring codons. Therefore, in some preferred embodiments, codons preferred by a particular prokaryotic or eukaryotic host (Murray et al., Nucl. Acids Res., 17: 477-498, 1989, herein incorporated by reference) are selected, for example, to increase the rate of JY-1 expression or to produce recombinant RNA transcripts having desirable properties, such as a longer half-life, than transcripts produced from naturally occurring sequence.

A. Vectors for Production of JY-1.

The polynucleotides of the present invention may be employed for producing polypeptides by recombinant techniques. Thus, for example, the polynucleotide may be included in any one of a variety of expression vectors for expressing a polypeptide. In some embodiments of the present invention, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In particular, some embodiments of the present invention provide recombinant constructs comprising one or more of the sequences as broadly described above (e.g., SEQ ID NOs: 1, 7 or 9). In some embodiments of the present invention, the constructs comprise a vector, such as a plasmid or viral vector, into which a sequence of the invention has been inserted, in a forward or reverse orientation. In still other embodiments, the heterologous structural sequence (e.g., SEQ ID NOs: 1, 7 or 9) is assembled in appropriate phase with translation initiation and termination sequences. In preferred embodiments of the present invention, the appropriate DNA sequence is inserted into the vector using any of a variety of procedures. In general, the DNA sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial—pQE70, pQE60, pQE-9 (Qiagen), pBS, pD10, phagescript, psiX174, pbluescript SK, pBSKS, pNH8A, pNH16a, pNH18A, pNH46A (Stratagene); ptrc99a, pKK223-3, pKK233-3, pDR540, pRIT5 (Pharmacia); 2) Eukaryotic—pWLNEO, pSV2CAT, pOG44, PXT1, pSG (Stratagene) pSVK3, pBPV, pMSG, pSVL (Pharmacia); and 3) Baculovirus—pPbac and pMbac (Stratagene). Any other plasmid or vector may be used as long as they are replicable and viable in the host. In some preferred embodiments of the present invention, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements.

In certain embodiments of the present invention, the DNA sequence in the expression vector is operatively linked to an appropriate expression control sequence(s) (promoter) to direct mRNA synthesis. Promoters useful in the present invention include, but are not limited to, the LTR or SV40 promoter, the E. coli lac or trp, the phage lambda P_{L} and P_{R}, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (e.g., dihydrofolate reductase or neomycin resistance for eukaryotic cell culture, or tetracycline or ampicillin resistance in E. coli).

In some embodiments of the present invention, transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin bp 100 to 270, a cytomegalovirus early promoter enhancer, the polyoma enhancer on the late side of the replication origin, and adenovirus enhancers.

In other embodiments, the expression vector also contains a ribosome binding site for translation initiation and a transcription terminator. In still other embodiments of the present invention, the vector may also include appropriate sequences for amplifying expression.

B. Host Cells for Production of JY-1.

In a further embodiment, the present invention provides host cells containing the above-described constructs. In some embodiments of the present invention, the host cell is a higher eukaryotic cell (e.g., a mammalian or insect cell). In other embodiments of the present invention, the host cell is a lower eukaryotic cell (e.g., a yeast cell). In still other embodiments of the present invention, the host cell can be a prokaryotic cell (e.g., a bacterial cell). Specific examples of host cells include, but are not limited to, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, (Gluzman, Cell 23:175-1981, herein incorporated by reference), C127, 3T3, 293, 293T, HeLa and BHK cell lines.

The constructs in host cells can be used in a conventional manner to produce the gene product encoded by the recombinant sequence. In some embodiments, introduction of the construct into the host cell can be accomplished by calcium phosphate transfection, DEAE-Dextran mediated transfection, or electroporation (See e.g., Davis et al., Basic Methods in Molecular Biology, [1986], herein incorporated by reference). Alternatively, in some embodiments of the present invention, the polypeptides of the invention can be synthetically produced by conventional peptide synthesizers.

Proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., [1989], herein incorporated by reference.

In some embodiments of the present invention, following transformation of a suitable host strain and growth of the host strain to an appropriate cell density, the selected promoter is induced by appropriate means (e.g., temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

C. Purification of JY-1.

The present invention also provides methods for recovering and purifying JY-1 from recombinant cell cultures including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. In other embodiments of the present invention, protein-refolding steps can be used as necessary, in completing configuration of the mature protein. In still other embodiments of the present invention, high performance liquid chromatography (HPLC) can be employed for final purification steps.

The present invention further provides polynucleotides having the coding sequence (e.g., SEQ ID NO: 8) fused in frame to a marker sequence that allows for purification of the polypeptide of the present invention. A non-limiting example of a marker sequence is a hexahistidine tag which may be supplied by a vector, preferably a pQE-9 vector, which provides for purification of the polypeptide fused to the marker in the case of a bacterial host, or, for example, the marker sequence may be a hemagglutinin (HA) tag when a mammalian host (e.g., COS-7 cells) is used. The HA tag corresponds to an epitope derived from the influenza hemagglutinin protein (Wilson et al., Cell, 37:767 [1984], herein incorporated by reference).

D. Truncation Mutants of JY-1.

In addition, the present invention provides fragments of JY-1 (i.e., truncation mutants). In some embodiments of the present invention, when expression of a portion of the JY-1 protein is desired, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al., J. Bacteriol., 169:751 [1987], herein incorporated by reference) and Salmonella typhimurium and its in vitro activity has been demonstrated on recombinant proteins (Miller et al., Proc. Natl. Acad. Sci. USA 84:2718 [1990], herein incorporated by reference). Therefore, removal of an N-terminal methionine, if desired, can be achieved either in vivo by expressing such recombinant polypeptides in a host which produces MAP (e.g., E. coli or CM89 or S. cerivisiae), or in vitro by use of purified MAP.

E. Fusion Proteins Containing JY-1.

The present invention also provides fusion proteins incorporating all or part of JY-1. Accordingly, in some embodiments of the present invention, the coding sequences for the polypeptide can be incorporated as a part of a fusion gene including a nucleotide sequence encoding a different polypeptide. It is contemplated that this type of expression system will find use under conditions where it is desirable to produce an immunogenic fragment of a JY-1 protein. In some embodiments of the present invention, the VP6 capsid protein of rotavirus is used as an immunologic carrier protein for portions of the JY-1 polypeptide, either in the monomeric form or in the form of a viral particle. In other embodiments of the present invention, the nucleic acid sequences corresponding to the portion of JY-1 against which antibodies are to be raised can be incorporated into a fusion gene construct which includes coding sequences for a late vaccinia virus structural protein to produce a set of recombinant viruses expressing fusion proteins comprising a portion of JY-1 as part of the virion. It has been demonstrated with the use of immunogenic fusion proteins utilizing the hepatitis B surface antigen fusion proteins that recombinant hepatitis B virions can be utilized in this role as well. Similarly, in other embodiments of the present invention, chimeric constructs coding for fusion proteins containing a portion of JY-1 and the poliovirus capsid protein are created to enhance immunogenicity of the set of polypeptide antigens (See e.g., EP Publication No. 025949; and Evans et al., Nature 339:385 [1989]; Huang et al., J. Virol., 62:3855 [1988]; and Schlienger et al., J. Virol., 66:2 [1992], all of which are herein incorporated by reference).

In still other embodiments of the present invention, the multiple antigen peptide system for peptide-based immunization can be utilized. In this system, a desired portion of JY-1 is obtained directly from organo-chemical synthesis of the peptide onto an oligomeric branching lysine core (see e.g., Posnett et al., J. Biol. Chem., 263:1719 [1988], herein incorporated by reference; and Nardelli et al., J. Immunol., 148:914 [1992], herein incorporated by reference). In other embodiments of the present invention, antigenic determinants of the JY-1 proteins can also be expressed and presented by bacterial cells.

In addition to utilizing fusion proteins to enhance immunogenicity, it is widely appreciated that fusion proteins can also facilitate the expression of proteins, such as the JY-1 protein of the present invention. Accordingly, in some embodiments of the present invention, JY-1 can be generated as a glutathione-S-transferase (i.e., GST fusion protein). It is contemplated that such GST fusion proteins will enable easy purification of JY-1, such as by the use of glutathione-derivatized matrices (See e.g., Ausabel et al. (eds.), Current Protocols in Molecular Biology, John Wiley & Sons, NY [1991], herein incorporated by reference). In another embodiment of the present invention, a fusion gene coding for a purification leader sequence, such as a poly-(His)/enterokinase cleavage site sequence at the N-terminus of the desired portion of JY-1, can allow purification of the expressed JY-1 fusion protein by affinity chromatography using a Ni²⁺ metal resin. In still another embodiment of the present invention, the purification leader sequence can then be subsequently removed by treatment with enterokinase (See e.g., Hochuli et al., J. Chromatogr., 411:177[1987]; and Janknecht et al., Proc. Natl. Acad. Sci. USA 88:8972, all of which are herein incorporated by reference).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment of the present invention, the fusion gene can be synthesized by conventional techniques including automated DNA synthesizers. Alternatively, in other embodiments of the present invention, PCR amplification of gene fragments can be carried out using anchor primers which give rise to complementary overhangs between two consecutive gene fragments which can subsequently be annealed to generate a chimeric gene sequence (See e.g., Current Protocols in Molecular Biology, supra).

F. Variants of JY-1.

Still other embodiments of the present invention provide mutant or variant forms of JY-1 (i.e., muteins). It is possible to modify the structure of a peptide having an activity of JY-1 for such purposes as enhancing therapeutic or prophylactic efficacy, or stability (e.g., ex vivo shelf life, and/or resistance to proteolytic degradation in vivo). Such modified peptides are considered functional equivalents of peptides having an activity of the subject JY-1 proteins as defined herein. A modified peptide can be produced in which the amino acid sequence has been altered, such as by amino acid substitution, deletion, or addition.

Moreover, as described above, variant forms (e.g., mutants or polymorphic sequences) of the subject JY-1 proteins are also contemplated as being equivalent to those peptides and DNA molecules that are set forth in more detail. For example, as described above, the present invention encompasses mutant and variant proteins that contain conservative or non-conservative amino acid substitutions.

This invention further contemplates a method of generating sets of combinatorial mutants of the present JY-1 proteins, as well as truncation mutants, and is especially useful for identifying potential variant sequences (i.e., mutants or polymorphic sequences). The purpose of screening such combinatorial libraries is to generate, for example, novel JY-1 variants that can act as either agonists or antagonists, or alternatively, possess novel activities all together.

Therefore, in some embodiments of the present invention, JY-1 variants are engineered by the present method to provide altered (e.g., increased or decreased) biological activity. In other embodiments of the present invention, combinatorially-derived variants are generated which have a selective potency relative to a naturally occurring JY-1. Such proteins, when expressed from recombinant DNA constructs, can be used in gene therapy protocols.

Still other embodiments of the present invention provide JY-1 variants that have intracellular half-lives dramatically different than the corresponding wild-type protein. For example, the altered protein can be rendered either more stable or less stable to proteolytic degradation or other cellular process that result in destruction of, or otherwise inactivate JY-1. Such variants, and the genes which encode them, can be utilized to alter the location of JY-1 expression by modulating the half-life of the protein. For instance, a short half-life can give rise to more transient JY-1 biological effects and, when part of an inducible expression system, can allow tighter control of JY-1 levels within the cell. As above, such proteins, and particularly their recombinant nucleic acid constructs, can be used in gene therapy protocols.

In still other embodiments of the present invention, JY-1 variants are generated by the combinatorial approach to act as antagonists, in that they are able to interfere with the ability of the corresponding wild-type protein to regulate cell function.

In some embodiments of the combinatorial mutagenesis approach of the present invention, the amino acid sequences for a population of JY-1 homologs, variants or other related proteins are aligned, preferably to promote the highest homology possible. Such a population of variants can include, for example, JY-1 homologs from one or more species, or JY-1 variants from the same species but which differ due to mutation or polymorphisms. Amino acids that appear at each position of the aligned sequences are selected to create a degenerate set of combinatorial sequences.

In a preferred embodiment of the present invention, the combinatorial JY-1 library is produced by way of a degenerate library of genes encoding a library of polypeptides which each include at least a portion of potential JY-1 protein sequences. For example, a mixture of synthetic oligonucleotides can be enzymatically ligated into gene sequences such that the degenerate set of potential JY-1 sequences are expressible as individual polypeptides, or alternatively, as a set of larger fusion proteins (e.g., for phage display) containing the set of JY-1 sequences therein.

There are many ways by which the library of potential JY-1 homologs and variants can be generated from a degenerate oligonucleotide sequence. In some embodiments, chemical synthesis of a degenerate gene sequence is carried out in an automatic DNA synthesizer, and the synthetic genes are ligated into an appropriate gene for expression. The purpose of a degenerate set of genes is to provide, in one mixture, all of the sequences encoding the desired set of potential JY-1 sequences. The synthesis of degenerate oligonucleotides is well known in the art (See e.g., Narang, Tetrahedron Lett., 39:39 [1983]; Itakura et al., Recombinant DNA, in Walton (ed.), Proceedings of the 3rd Cleveland Symposium on Macromolecules, Elsevier, Amsterdam, pp 273-289 [1981]; Itakura et al., Annu. Rev. Biochem., 53:323 [1984]; Itakura et al., Science 198:1056 [1984]; Ike et al., Nucl. Acid Res., 11:477 [1983]; each of which is incorporated herein by reference). Such techniques have been employed in the directed evolution of other proteins (See e.g., Scott et al., Science 249:386 [1980]; Roberts et al., Proc. Natl. Acad. Sci. USA 89:2429 [1992]; Devlin et al., Science 249: 404 [1990]; Cwirla et al, Proc. Natl. Acad. Sci. USA 87: 6378 [1990]; each of which is herein incorporated by reference; as well as U.S. Pat. Nos. 5,223,409, 5,198,346, and 5,096,815; each of which is incorporated herein by reference).

It is contemplated that the JY-1 nucleic acids (e.g., SEQ ID NOs: 1, 7 and 9, and fragments and variants thereof) can be utilized as starting nucleic acids for directed evolution. These techniques can be utilized to develop JY-1 variants having desirable properties such as increased or decreased biological activity.

In some embodiments, artificial evolution is performed by random mutagenesis (e.g., by utilizing error-prone PCR to introduce random mutations into a given coding sequence). This method requires that the frequency of mutation be finely tuned. As a general rule, beneficial mutations are rare, while deleterious mutations are common. This is because the combination of a deleterious mutation and a beneficial mutation often results in an inactive enzyme. The ideal number of base substitutions for targeted gene is usually between 1.5 and 5 (Moore and Arnold, Nat. Biotech., 14, 458 [1996]; Leung et al., Technique, 1:11 [1989]; Eckert and Kunkel, PCR Methods Appl., 1:17-24 [1991]; Caldwell and Joyce, PCR Methods Appl., 2:28 [1992]; and Zhao and Arnold, Nuc. Acids. Res., 25:1307 [1997], each of which are herein incorporated by reference). After mutagenesis, the resulting clones are selected for desirable activity (e.g., screened for JY-1 activity). Successive rounds of mutagenesis and selection are often necessary to develop enzymes with desirable properties. It should be noted that only the useful mutations are carried over to the next round of mutagenesis.

In other embodiments of the present invention, the polynucleotides of the present invention are used in gene shuffling or sexual PCR procedures (e.g., Smith, Nature, 370:324 [1994]; U.S. Pat. Nos. 5,837,458; 5,830,721; 5,811,238; 5,733,731; all of which are herein incorporated by reference). Gene shuffling involves random fragmentation of several mutant DNAs followed by their reassembly by PCR into full-length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DNaseI and subjected to multiple rounds of PCR with no added primer. The lengths of random fragments approach that of the uncleaved segment as the PCR cycles proceed, resulting in mutations in present in different clones becoming mixed and accumulating in some of the resulting sequences. Multiple cycles of selection and shuffling have led to the functional enhancement of several enzymes (Stemmer, Nature, 370:398 [1994]; Stemmer, Proc. Natl. Acad. Sci. USA, 91:10747 [1994]; Crameri et al., Nat. Biotech., 14:315 [1996]; Zhang et al., Proc. Natl. Acad. Sci. USA, 94:4504 [1997]; and Crameri et al., Nat. Biotech., 15:436 [1997], each of which are herein incorporated by reference). Variants produced by directed evolution can be screened for JY-1 activity by the methods described herein.

A wide range of techniques are known in the art for screening gene products of combinatorial libraries made by point mutations, and for screening cDNA libraries for gene products having a certain property. Such techniques will be generally adaptable for rapid screening of the gene libraries generated by the combinatorial mutagenesis or recombination of JY-1 homologs or variants. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

G. Chemical Synthesis of JY-1.

In an alternate embodiment of the invention, the coding sequence of JY-1 is synthesized, whole or in part, using chemical methods well known in the art (See e.g., Caruthers et al., Nucl. Acids Res. Symp. Ser., 7:215 [1980]; Crea and Horn, Nucl. Acids Res., 9:2331 [1980]; Matteucci and Caruthers, Tetrahedron Lett., 21:719 [1980]; and Chow and Kempe, Nucl. Acids Res., 9:2807 [1981], each of which are herein incorporated by reference). In other embodiments of the present invention, the protein itself is produced using chemical methods to synthesize either an entire JY-1 amino acid sequence or a portion thereof. For example, peptides can be synthesized by solid phase techniques, cleaved from the resin, and purified by preparative high performance liquid chromatography (See e.g., Creighton, Proteins Structures And Molecular Principles, W H Freeman and Co, New York N.Y. [1983], herein incorporated by reference). In other embodiments of the present invention, the composition of the synthetic peptides is confirmed by amino acid analysis or sequencing (See e.g., Creighton, supra).

Direct peptide synthesis can be performed using various solid-phase techniques (Roberge et al, Science 269:202 [1995], herein incorporated by reference) and automated synthesis may be achieved, for example, using ABI 431A Peptide Synthesizer (Perkin Elmer) in accordance with the instructions provided by the manufacturer. Additionally, the amino acid sequence of JY-1, or any part thereof, may be altered during direct synthesis and/or combined using chemical methods with other sequences to produce a variant polypeptide.

### III. Detection of JY-1 Alleles.

In some embodiments, the present invention provides methods of detecting the presence of wild or variant (e.g., mutant or polymorphic) JY-1 nucleic acids or polypeptides. The detection of mutant JY-1 polypeptides finds use in the diagnosis of disease).

A. Detection of JY-1 Alleles.

Accordingly, the present invention provides methods for determining whether a patient has a variant JY-1 allele. A number of methods are available for analysis of variant (e.g., mutant or polymorphic) nucleic acid sequences. Assays for detection variants (e.g., polymorphisms or mutations) fall into several categories, including, but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention.

1. Direct Sequencing Assays.

In some embodiments of the present invention, variant sequences are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, or automated sequencing. The results of the sequencing are displayed using any suitable method. The sequence is examined and the presence or absence of a given SNP or mutation is determined.

2. PCR Assay.

In some embodiments of the present invention, variant sequences are detected using a PCR-based assay. In some embodiments, the PCR assay comprises the use of oligonucleotide primers that specifically hybridize to the variant or wild type allele of JY-1 (e.g., to the region of polymorphism or mutation). Both sets of primers are used to amplify a sample of DNA. If the mutant primers result in a PCR product, then the patient has the mutant JY-1 allele. If the wild-type primers result in a PCR product, then the patient has the wild type allele of JY-1.

3. Mutational Detection by dHPLC.

In some embodiments of the present invention, variant sequences are detected using a PCR-based assay with consecutive detection of nucleotide variants by dHPLC (denaturing high performance liquid chromatography). Exemplary systems and Methods for dHPLC include, but are not limited to, WAVE (Transgenomic, Inc; Omaha, Nebr.) or VARIAN equipment (Palo Alto, Calif.).

4. Fragment Length Polymorphism Assays.

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I [Third Wave Technologies, Madison, Wis.] enzyme). DNA fragments from a sample containing a SNP or a mutation will have a different banding pattern than wild type.

a. RFLP Assay.

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from wild-type and mutant controls.

b. CFLP Assay.

In other embodiments, variant sequences are detected using a CLEAVASE fragment length polymorphism assay (CFLP; Third Wave Technologies, Madison, Wis.; See e.g., U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

5. Hybridization Assays.

In preferred embodiments of the present invention, variant sequences are detected a hybridization assay. In a hybridization assay, the presence of absence of a given SNP or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

a. Direct Detection of Hybridization.

In some embodiments, hybridization of a probe to the sequence of interest (e.g., a SNP or mutation) is detected directly by visualizing a bound probe (e.g., a Northern or Southern assay; See e.g., Ausabel et al. (eds.), Current Protocols in Molecular Biology, John Wiley & Sons, NY [1991], herein incorporated by reference). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition or low, medium, or high stringency conditions. Unbound probe is removed and the presence of binding is detected by visualizing the labeled probe.

b. Detection of Hybridization Using “DNA Chip” Assays.

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered primarily to the sites where amidite A is to be coupled during that synthesis step and so on. Common reagents and washes are delivered by flooding the entire surface and then removing them by spinning.

DNA probes unique for the SNP or mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

c. Enzymatic Detection of Hybridization.

In some embodiments of the present invention, hybridization is detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified genomic DNA. The isolated DNA sample is contacted with the first probe specific either for a SNP/mutation or wild type sequence and allowed to hybridize. Then a secondary probe, specific to the first probe, and containing the fluorescein label, is hybridized and the enzyme is added. Binding is detected by using a fluorescent plate reader and comparing the signal of the test sample to known positive and negative controls.

In some embodiments, hybridization of a bound probe is detected using a TaqMan assay (PE Biosystems, Foster City, Calif.; See e.g., U.S. Pat. Nos. 5,962,233 and 5,538,848, each of which is herein incorporated by reference). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin).

6. Mass Spectroscopy Assay.

In some embodiments, a MassARRAY system (Sequenom, San Diego, Calif.) is used to detect variant sequences (See e.g., U.S. Pat. Nos. 6,043,031; 5,777,324; and 5,605,798; each of which is herein incorporated by reference). DNA is isolated from blood samples using standard procedures. Next, specific DNA regions containing the mutation or SNP of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization—Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

7. Detection of Variant JY-1 Proteins.

In other embodiments, variant JY-1 polypeptides are detected (e.g., including, but not limited to, those encoded by SEQ ID NOs: 8, 10-29). Any suitable method may be used to detect truncated or mutant JY-1 polypeptides including, but not limited to, those described below.

In some embodiments of the present invention, antibodies (See below for antibody production) are used to determine if an individual contains an allele encoding a variant JY-1 gene. In preferred embodiments, antibodies are utilized that discriminate between variant (i.e., truncated proteins); and wild-type proteins (SEQ ID NO:8). In some particularly preferred embodiments, the antibodies are directed to the C-terminus of JY-1. Proteins that are recognized by the N-terminal, but not the C-terminal antibody are truncated. In some embodiments, quantitative immunoassays are used to determine the ratios of C-terminal to N-terminal antibody binding. In other embodiments, antibodies that differentially bind to wild type or variant forms of JY-1.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference.

In some embodiments, the analysis and presentation of results is also automated. For example, in some embodiments, software that generates a prognosis based on the result of the immunoassay is utilized.

In other embodiments, the immunoassay described in U.S. Pat. Nos. 5,599,677 and 5,672,480; each of which is herein incorporated by reference.

8. Kits.

The present invention also provides kits for determining whether a cell or tissue is expressing a JY-1 gene, such as an antibody, a PCR primer set, a JY-1 gene fragment, constructs for inhibiting JY-1 expression, etc. The present invention also provides kits for determining whether an individual contains a wild-type or variant (e.g., mutant or polymorphic) allele of JY-1. The diagnostic kits are contemplated in a variety of ways. In some embodiments, the kits contain at least one reagent for specifically detecting a JY-1 allele. In one embodiment, the kit contains at least one reagent for specifically detecting a JY-1 protein. In one embodiment, the kit contains an oligonucleotide reagent for detecting an expressed JY-1 cDNA. In some embodiments, the kits contain at least one reagent for specifically detecting a mutant JY-1 allele or protein. In one embodiment, the kits contain reagents for detecting a truncation in the JY-1 gene. In preferred embodiments, the reagent is a nucleic acid that hybridizes to nucleic acids containing the mutation and that does not bind to nucleic acids that do not contain the mutation. In other preferred embodiments, the reagents are primers for amplifying the region of DNA containing the mutation. In still other embodiments, the reagents are antibodies that preferentially bind to a JY-1 protein. In still other embodiments the antibody reagents distinguish between a wild-type or truncated JY-1 or mutated JY-1 protein.

In some embodiments, the kits include ancillary reagents such as buffering agents, nucleic acid stabilizing reagents, protein stabilizing reagents, and signal producing systems (e.g., florescence generating systems as Fret systems). The test kit may be packages in any suitable manner, typically with the elements in a single container or various containers as necessary along with a sheet of instructions for carrying out the test. In some embodiments, the kits also preferably include a positive control sample.

### IV. Generation of JY-1 Antibodies.

The present invention provides serum antibodies, isolated antibodies, and contemplates antibody fragments (e.g., Fab′ fragments). Antibodies were generated as described herein, for the detection of a JY-1 protein. The antibodies may be prepared using various immunogens. In one embodiment, the immunogen is a human JY-1 peptide to generate antibodies that recognize human JY-1. Such antibodies include, but are not limited to polyclonal, monoclonal, chimeric, single chain, Fab fragments, Fab expression libraries, or recombinant (e.g., chimeric, humanized, etc.) antibodies, as long as it can recognize the protein. Antibodies can be produced by using a protein of the present invention as the antigen according to a conventional antibody or antiserum preparation process.

Various procedures known in the art may be used for the production of polyclonal antibodies directed against JY-1. For the production of antibody, various host animals can be immunized by injection with the peptide corresponding to the JY-1 epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies directed toward JY-1, it is contemplated that any technique that provides for the production of antibody molecules by continuous cell lines in culture will find use with the present invention (See e.g., Harlow and Lane, Antibodies: A Laboratory Manual, Cold Spring Harbor Laboratory Press, Cold Spring Harbor, N.Y.; herein incorporated by reference). These include but are not limited to the hybridoma technique originally developed by Köhler and Milstein (Köhler and Milstein, Nature 256:495-497 [1975]; herein incorporated by reference), as well as the trioma technique, the human B-cell hybridoma technique (See e.g., Kozbor et al., Immunol. Tod., 4:72[1983]; herein incorporated by reference), and the EBV-hybridoma technique to produce human monoclonal antibodies (Cole et al., in Monoclonal Antibodies and Cancer Therapy, Alan R. Liss, Inc., pp. 77-96 [1985]; herein incorporated by reference).

In an additional embodiment of the invention, monoclonal antibodies are produced in germ-free animals utilizing technology such as that described in PCT/US90/02545; herein incorporated by reference). Furthermore, it is contemplated that human antibodies will be generated by human hybridomas (Cote et al., Proc. Natl. Acad. Sci. USA 80:2026-2030 [1983], herein incorporated by reference) or by transforming human B cells with EBV virus in vitro (Cole et al., in Monoclonal Antibodies and Cancer Therapy, Alan R. Liss, pp. 77-96 [1985]; herein incorporated by reference).

In addition, it is contemplated that techniques described for the production of single chain antibodies (U.S. Pat. No. 4,946,778; herein incorporated by reference) will find use in producing JY-1 specific single chain antibodies. An additional embodiment of the invention utilizes the techniques described for the construction of Fab expression libraries (Huse et al., Science 246:1275-1281 [1989]; herein incorporated by reference) to allow rapid and easy identification of monoclonal Fab fragments with the desired specificity for JY-1.

In other embodiments, the present invention contemplated recombinant antibodies or fragments thereof to the proteins of the present invention. Recombinant antibodies include, but are not limited to, humanized and chimeric antibodies. Methods for generating recombinant antibodies are known in the art (See e.g., U.S. Pat. Nos. 6,180,370 and 6,277,969 and “Monoclonal Antibodies” H. Zola, BIOS Scientific Publishers Limited 2000. Springer-Verlay New York, Inc., New York; each of which is herein incorporated by reference).

It is contemplated that any technique suitable for producing antibody fragments will find use in generating antibody fragments that contain the idiotype (antigen binding region) of the antibody molecule. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent.

In the production of antibodies, it is contemplated that screening for the desired antibody will be accomplished by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.).

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. As is well known in the art, the immunogenic peptide should be provided free of the carrier molecule used in any immunization protocol. For example, if the peptide was conjugated to KLH, it may be conjugated to BSA, or used directly, in a screening assay).

The foregoing antibodies can be used in methods known in the art relating to the localization and structure of JY-1 (e.g., for Western blotting), measuring levels thereof in appropriate biological samples, etc. The antibodies can be used to detect JY-1 in a biological sample from an individual. The biological sample can be a biological fluid, such as, but not limited to, blood, serum, plasma, interstitial fluid, urine, cerebrospinal fluid, follicular fluid and the like, containing cells.

The biological samples can then be tested directly for the presence of human JY-1 using an appropriate strategy (e.g., ELISA or radioimmunoassay) and format (e.g., microwells, dipstick (e.g., as described in International Patent Publication WO 93/03367, herein incorporated by reference), etc. Alternatively, proteins in the sample can be size separated (e.g., by polyacrylamide gel electrophoresis (PAGE), in the presence or not of sodium dodecyl sulfate (SDS), and the presence of JY-1 detected by immunoblotting (Western blotting). Immunoblotting techniques are generally more effective with antibodies generated against a peptide corresponding to an epitope of a protein, and hence, are particularly suited to the present invention.

V. Transgenic Animals Expressing Exogenous JY-1 Genes and Homologs, Mutants, and Variants thereof.

The present invention contemplates the generation of transgenic animals comprising an exogenous JY-1 gene or homologs, mutants, or variants thereof. In preferred embodiments, the transgenic animal displays an altered phenotype as compared to wild-type animals. In some embodiments, the altered phenotype is the overexpression of mRNA for a JY-1 gene as compared to wild-type levels of JY-1 expression. In other embodiments, the altered phenotype is the decreased expression of mRNA for an endogenous JY-1 gene as compared to wild-type levels of endogenous JY-1 expression. In some preferred embodiments, the transgenic animals comprise mutant (e.g., truncated) alleles of JY-1. Methods for analyzing the presence or absence of such phenotypes include Northern blotting, mRNA protection assays, and RT-PCR. In other embodiments, the transgenic mice have a knock out mutation of the JY-1 gene.

Such animals find use in research applications (e.g., identifying signaling pathways that JY-1 is involved in), as well as drug screening applications. For example, in some embodiments, test compounds (e.g., compounds suspected of affecting follicular development) and control compounds (e.g., a placebo) are administered to the transgenic animals and the control animals and the effects evaluated. The effects of the test and control compounds on disease symptoms are then assessed.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter, which allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]; herein incorporated by reference). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191; herein incorporated by reference, describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]; herein incorporated by reference). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986], each of which are herein incorporated by reference). The viral vector system used to introduce the transgene is typically a replication-defective retrovirus carrying the transgene (Jahner, et al., Proc. Natl. Acad. Sci. USA 82:6927 [1985]; herein incorporated by reference). Transfection is easily and efficiently obtained by culturing the blastomeres on a monolayer of virus-producing cells (Van der Putten, supra; Stewart, et al., EMBO J., 6:383 [1987]; herein incorporated by reference). Alternatively, infection can be performed at a later stage. Virus or virus-producing cells can be injected into the blastocoele (Jahner, et al., Nature 298:623 [1982]; herein incorporated by reference). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner, et al., supra [1982]; herein incorporated by reference). Additional means of using retroviruses or retroviral vectors to create transgenic animals known to the art involves the micro-injection of retroviral particles or mitomycin C-treated cells producing retrovirus into the perivitelline space of fertilized eggs or early embryos (PCT International Application WO 90/08832 [1990]; herein incorporated by reference, and Haskell and Bowen, Mol. Reprod. Dev., 40:386[1995]; herein incorporated by reference).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]; all of which are herein incorporated by reference). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]; herein incorporated by reference). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, the transgene is introduced into somatic cells, and the somatic cell (either quiescent or proliferating) is fused to an enucleated oocyte via nuclear transfer. Thus, the transgene is introduced into the enucleated oocyte. These procedures are described in detail in U.S. Pat. Nos. 5,945,577; 6,147,276; and 6,235,969, all of which are incorporated herein by reference.

In still other embodiments, homologous recombination is utilized to knock-out gene function or create deletion mutants (e.g., mutants in which the LRRs of JY-1 are deleted). Methods for homologous recombination are described in U.S. Pat. No. 5,614,396, incorporated herein by reference.

### VI. Drug Screening Using JY-1.

In some embodiments, the isolated nucleic acid sequences of JY-1 (e.g., SEQ ID NOs: 1, 7 and 9) are used in drug screening applications for compounds that alter (e.g., enhance) JY-1 activity.

A. Identification of Binding Partners.

In some embodiments, binding partners of JY-1 amino acids are identified. In some embodiments, the JY-1 nucleic acid sequence (e.g., SEQ ID NOs: 1, 7 and 9) or fragments thereof are used in yeast two-hybrid screening assays. For example, in some embodiments, the nucleic acid sequences are subcloned into pGPT9 (Clontech, La Jolla, Calif.) to be used as a bait in a yeast-2-hybrid screen for protein-protein interaction of mammalian (e.g., human, primate or bovine) ovary cDNA library (Fields and Song Nature 340:245-246, 1989; herein incorporated by reference). In other embodiments, phage display is used to identify binding partners (Parmley and Smith Gene 73:305-318, [1988]; herein incorporated by reference).

B. Drug Screening.

The present invention provides methods and compositions for using JY-1 as a target for screening drugs that can alter, for example, interaction between JY-1 and JY-1 binding partners (e.g., those identified using the above methods).

In one screening method, the two-hybrid system is used to screen for compounds (e.g., drug) capable of altering (e.g., inhibiting) JY-1 function(s) (e.g., interaction with a binding partner) in vitro or in vivo. In one embodiment, a GAL4 binding site, linked to a reporter gene such as lacZ, is contacted in the presence and absence of a candidate compound with a GAL4 binding domain linked to a JY-1 fragment and a GAL4 transactivation domain II linked to a binding partner fragment. Expression of the reporter gene is monitored and a decrease in the expression is an indication that the candidate compound inhibits the interaction of JY-1 with the binding partner. Alternately, the effect of candidate compounds on the interaction of JY-1 with other proteins (e.g., proteins known to interact directly or indirectly with the binding partner) can be tested in a similar manner.

In another screening method, candidate compounds are evaluated for their ability to alter JY-1 signaling by contacting JY-1, binding partners, binding partner-associated proteins, or fragments thereof, with the candidate compound and determining binding of the candidate compound to the peptide. The protein or protein fragments is/are immobilized using methods known in the art such as binding a GST-JY-1 fusion protein to a polymeric bead containing glutathione. A chimeric gene encoding a GST fusion protein is constructed by fusing DNA encoding the polypeptide or polypeptide fragment of interest to the DNA encoding the carboxyl terminus of GST (See e.g., Smith et al., Gene 67:31 [1988]; herein incorporated by reference). The fusion construct is then transformed into a suitable expression system (e.g., E. coli XA90) in which the expression of the GST fusion protein can be induced with isopropyl-β-D-thiogalactopyranoside (IPTG). Induction with IPTG should yield the fusion protein as a major constituent of soluble, cellular proteins. The fusion proteins can be purified by methods known to those skilled in the art, including purification by glutathione affinity chromatography. Binding of the candidate compound to the proteins or protein fragments is correlated with the ability of the compound to disrupt the signal transduction pathway and thus regulate JY-1 physiological effects.

In another screening method, one of the components of the JY-1/binding partner signaling system, is immobilized. Polypeptides can be immobilized using methods known in the art, such as adsorption onto a plastic microtiter plate or specific binding of a GST-fusion protein to a polymeric bead containing glutathione. For example, GST-JY-1 is bound to glutathione-Sepharose beads. The immobilized peptide is then contacted with another peptide with which it is capable of binding in the presence and absence of a candidate compound. Unbound peptide is then removed and the complex solubilized and analyzed to determine the amount of bound labeled peptide. A decrease in binding is an indication that the candidate compound inhibits the interaction of JY-1 with the other peptide. A variation of this method allows for the screening of compounds that are capable of disrupting a previously-formed protein/protein complex. For example, in some embodiments a complex comprising JY-1 or a JY-1 fragment bound to another peptide is immobilized as described above and contacted with a candidate compound. The dissolution of the complex by the candidate compound correlates with the ability of the compound to disrupt or inhibit the interaction between JY-1 and the other peptide.

Another technique for drug screening provides high throughput screening for compounds having suitable binding affinity to JY-1 peptides and is described in detail in WO 84/03564, incorporated herein by reference. Briefly, large numbers of different small peptide test compounds are synthesized on a solid substrate, such as plastic pins or some other surface. The peptide test compounds are then reacted with JY-1 peptides and washed. Bound JY-1 peptides are then detected by methods well known in the art.

Another technique uses JY-1 antibodies, generated as discussed above. Such antibodies capable of specifically binding to JY-1 peptides compete with a test compound for binding to JY-1. In this manner, the antibodies can be used to detect the presence of any peptide that shares one or more antigenic determinants of the JY-1 peptide.

The present invention contemplates many other means of screening compounds. The examples provided above are presented merely to illustrate a range of techniques available. One of ordinary skill in the art will appreciate that many other screening methods can be used.

In particular, the present invention contemplates the use of cell lines transfected with JY-1 and variants thereof for screening compounds for activity, and in particular to high throughput screening of compounds from combinatorial libraries (e.g., libraries containing greater than 104 compounds). The cell lines of the present invention can be used in a variety of screening methods. In some embodiments, the cells can be used in second messenger assays that monitor signal transduction following activation of cell-surface receptors. In other embodiments, the cells can be used in reporter gene assays that monitor cellular responses at the transcription/translation level. In still further embodiments, the cells can be used in cell proliferation assays to monitor the overall growth/no growth response of cells to external stimuli.

In second messenger assays, the host cells are preferably transfected as described above with vectors encoding JY-1 or variants or mutants thereof. The host cells are then treated with a compound or plurality of compounds (e.g., from a combinatorial library) and assayed for the presence or absence of a response. It is contemplated that at least some of the compounds in the combinatorial library can serve as agonists, antagonists, activators, or inhibitors of the protein or proteins encoded by the vectors. It is also contemplated that at least some of the compounds in the combinatorial library can serve as agonists, antagonists, activators, or inhibitors of protein acting upstream or downstream of the protein encoded by the vector in a signal transduction pathway.

In some embodiments, the second messenger assays measure fluorescent signals from reporter molecules that respond to intracellular changes (e.g., Ca²⁺ concentration, membrane potential, pH, IP₃, cAMP, arachidonic acid release) due to stimulation of membrane receptors and ion channels (e.g., ligand gated ion channels; see Denyer et al., Drug Discov. Today 3:323 [1998]; and Gonzales et al., Drug. Discov. Today 4:431-39 [1999]; all of which are herein incorporated by reference). Examples of reporter molecules include, but are not limited to, FRET (florescence resonance energy transfer) systems (e.g., Cuo-lipids and oxonols, EDAN/DABCYL), calcium sensitive indicators (e.g., Fluo-3, FURA 2, INDO 1, and FLUO3/AM, BAPTA AM), chloride-sensitive indicators (e.g., SPQ, SPA), potassium-sensitive indicators (e.g., PBFI), sodium-sensitive indicators (e.g., SBFI), and pH sensitive indicators (e.g., BCECF).

In general, the host cells are loaded with the indicator prior to exposure to the compound. Responses of the host cells to treatment with the compounds can be detected by methods known in the art, including, but not limited to, fluorescence microscopy, confocal microscopy (e.g., FCS systems), flow cytometry, microfluidic devices, FLIPR systems (See, e.g., Schroeder and Neagle, J. Biomol. Screening 1:75 [1996]; herein incorporated by reference), and plate-reading systems. In some preferred embodiments, the response (e.g., increase in fluorescent intensity) caused by compound of unknown activity is compared to the response generated by a known agonist and expressed as a percentage of the maximal response of the known agonist. The maximum response caused by a known agonist is defined as a 100% response. Likewise, the maximal response recorded after addition of an agonist to a sample containing a known or test antagonist is detectably lower than the 100% response.

The cells are also useful in reporter gene assays. Reporter gene assays involve the use of host cells transfected with vectors encoding a nucleic acid comprising transcriptional control elements of a target gene (i.e., a gene that controls the biological expression and function of a disease target) spliced to a coding sequence for a reporter gene. Therefore, activation of the target gene results in activation of the reporter gene product. In some embodiments, the reporter gene construct comprises the 5′ regulatory region (e.g., promoters and/or enhancers) of a protein whose expression is controlled by JY-1 in operable association with a reporter gene (See Example 4 and Inohara et al., J. Biol. Chem. 275:27823 [2000], herein incorporated by reference, for a description of the luciferase reporter construct pBVIx-Luc). Examples of reporter genes finding use in the present invention include, but are not limited to, chloramphenicol transferase, alkaline phosphatase, firefly and bacterial luciferases, β-galactosidase, β-lactamase, and green fluorescent protein. The production of these proteins, with the exception of green fluorescent protein, is detected through the use of chemiluminescent, calorimetric, or bioluminecent products of specific substrates (e.g., X-gal and luciferin). Comparisons between compounds of known and unknown activities may be conducted as described above.

Specifically, the present invention provides screening methods for identifying modulators, i.e., candidate or test compounds or agents (e.g., proteins, peptides, peptidomimetics, peptoids, small molecules or other drugs) which bind to JY-1 of the present invention, have an inhibitory (or stimulatory) effect on, for example, JY-1 expression or JY-1 activity, or have a stimulatory or inhibitory effect on, for example, the expression or activity of a JY-1 substrate. Compounds thus identified can be used to modulate the activity of target gene products (e.g., JY-1 genes) either directly or indirectly in a therapeutic protocol, to elaborate the biological function of the target gene product, or to identify compounds that disrupt normal target gene interactions. Compounds that stimulate the activity of a variant JY-1 or mimic the activity of a non-functional variant are particularly useful.

In one embodiment, the invention provides assays for screening candidate or test compounds that are substrates of a JY-1 protein or polypeptide or a biologically active portion thereof. In another embodiment, the invention provides assays for screening candidate or test compounds that bind to or modulate the activity of a JY-1 protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994], herein incorporated by reference); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145, herein incorporated by reference). Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994]; all of which are herein incorporated by reference. Libraries of compounds may be presented in solution (e.g., Houghten, Biotechniques 13:412-421 [1992], herein incorporated by reference), or on beads (Lam, Nature 354:82-84 [1991], herein incorporated by reference), chips (Fodor, Nature 364:555-556 [1993], herein incorporated by reference), bacteria or spores (U.S. Pat. No. 5,223,409; herein incorporated by reference), plasmids (Cull et al., Proc. Nad. Acad. Sci. USA 89:18651869 [1992], herein incorporated by reference) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]; all of which are herein incorporated by reference).

In one embodiment, an assay is a cell-based assay in which a cell that expresses a JY-1 protein or biologically active portion thereof is contacted with a test compound, and the ability of the test compound to the modulate JY-1's activity is determined. Determining the ability of the test compound to modulate JY-1 activity can be accomplished by monitoring, for example, changes in enzymatic activity. The cell, for example, can be of mammalian origin.

The ability of the test compound to modulate JY-1 binding to a compound, e.g., a JY-1 substrate, can also be evaluated. This can be accomplished, for example, by coupling the compound, e.g., the substrate, with a radioisotope or enzymatic label such that binding of the compound, e.g., the substrate, to a JY-1 can be determined by detecting the labeled compound, e.g., substrate, in a complex.

Alternatively, the JY-1 is coupled with a radioisotope or enzymatic label to monitor the ability of a test compound to modulate JY-1 binding to a JY-1 substrate in a complex. For example, compounds (e.g., substrates) can be labeled with ¹²⁵I, ³⁵S ¹⁴C or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a JY-1 substrate) to interact with a JY-1 with or without the labeling of any of the interactants can be evaluated. For example, a microphysiorneter can be used to detect the interaction of a compound with a JY-1 without the labeling of either the compound or the JY-1 (McConnell et al. Science 257:1906-1912 [1992]; herein incorporated by reference). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and JY-1.

In yet another embodiment, a cell-free assay is provided in which a JY-1 protein or biologically active portion thereof is contacted with a test compound and the ability of the test compound to bind to the JY-1 protein or biologically active portion thereof is evaluated. Preferred biologically active portions of the JY-1 proteins to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz, et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in 1 5 the assay should be maximal. An FRET binding event can be conveniently measured through standard fluorometric detection means well known in the art (e.g., using a fluorimeter).

In another embodiment, determining the ability of the JY-1 protein to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]; all of which are herein incorporated by reference). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize JY-1, an anti-JY-1 antibody or its target molecule to facilitate separation of complexed from non-complexed forms of one or both of the proteins, as well as to accommodate automation of the assay. Binding of a test compound to a JY-1 protein, or interaction of a JY-1 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-JY-1 fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or JY-1 protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of JY-1 binding or activity determined using standard techniques. Other techniques for immobilizing either JY-1 protein or a target molecule on matrices include using conjugation of biotin and streptavidin. Biotinylated JY-1 protein or target molecules can be prepared from biotin-NHS (N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with JY-1 protein or target molecules but which do not interfere with binding of the JY-1 protein to its target molecule. Such antibodies can be derivatized to the wells of the plate, and unbound target or JY-1 protein trapped in the wells by antibody conjugation. Methods for detecting such complexes, in addition to those described above for the GST-immobilized complexes, include immunodetection of complexes using antibodies reactive with the JY-1 protein or target molecule, as well as enzyme-linked assays which rely on detecting an enzymatic activity associated with the JY-1 protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993], herein incorporated by reference); chromatography (gel filtration chromatography, ion-exchange chromatography); electrophoresis (see, e.g., Ausubel et al., eds. Current Protocols in Molecular Biology 1999, J. Wiley: New York, herein incorporated by reference); and immunoprecipitation (see, for example, Ausubel et al., eds. Current Protocols in Molecular Biology 1999, J. Wiley: New York, herein incorporated by reference). Such resins and chromatographic techniques are known to one skilled in the art (See e.g., Heegaard, J. Mol. Recognit. 11:141-8 [1998]; Hageand, Chromatogr. Biomed. Sci. Appl 699:499-525 [1997]; all of which are herein incorporated by reference). Further, fluorescence energy transfer may also be conveniently utilized, as described herein, to detect binding without further purification of the complex from solution.

The assay can include contacting the JY-1 protein or biologically active portion thereof with a known compound that binds the JY-1 to form an assay mixture, contacting the assay mixture with a test compound, and determining the ability of the test compound to interact with a JY-1 protein, wherein determining the ability of the test compound to interact with a JY-1 protein includes determining the ability of the test compound to preferentially bind to JY-1 or biologically active portion thereof, or to modulate the activity of a target molecule, as compared to the known compound.

To the extent that JY-1 can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, JY-1 protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent WO 94/10300; each of which is herein incorporated by reference), to identify other proteins, that bind to or interact with JY-1 (“JY-1-binding proteins” or “JY-1-bp”) and are involved in JY-1 activity. Such JY-1-bps can be activators or inhibitors of signals by the JY-1 proteins or targets as, for example, downstream elements of a JY-1-mediated signaling pathway.

Modulators of JY-1 expression can also be identified. For example, a cell or cell free mixture is contacted with a candidate compound and the expression of JY-1 mRNA or protein evaluated relative to the level of expression of JY-1 mRNA or protein in the absence of the candidate compound. When expression of JY-1 mRNA or protein is greater in the presence of the candidate compound than in its absence, the candidate compound is identified as a stimulator of JY-1 mRNA or protein expression. Alternatively, when expression of JY-1 mRNA or protein is less (i.e., statistically significantly less) in the presence of the candidate compound than in its absence, the candidate compound is identified as an inhibitor of JY-1 mRNA or protein expression. The level of JY-1 mRNA or protein expression can be determined by methods described herein for detecting JY-1 mRNA or protein.

A modulating agent can be identified using a cell-based or a cell free assay, and the ability of the agent to modulate the activity of a JY-1 protein can be confirmed in vivo, e.g., in an animal such as an animal model for a disease.

C. Therapeutic Agents.

This invention further pertains to novel agents identified by the above-described screening assays. Accordingly, it is within the scope of this invention to further use an agent identified as described herein (e.g., a JY-1 modulating agent or mimetic, a JY-1 specific antibody, or a JY-1-binding partner) in an appropriate animal model (such as those described herein) to determine the efficacy, toxicity, side effects, or mechanism of action, of treatment with such an agent.

### VII. RNAi Reagents.

The present invention also contemplates the use of RNA interference (RNAi) to regulate levels of JY-1 within cells and provides RNAi probes for such use. In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs.

Carthew reported (Curr. Opin. Cell Biol. 13(2):244-248 (2001), herein incorporated by reference), that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

In preferred embodiments, the RNAi used to interfere with JY-1 polypeptide production is a small interfering RNA (siRNA), sometimes known as short interfering RNA or silencing RNA, are a class of 18-29 nucleotide-long double-stranded and single stranded RNA molecules.

In preferred embodiments, the dsRNA and ssRNA used to initiate RNAi, may be isolated from native source or produced by known means, e.g., transcribed from DNA. The promoters and vectors described in more detail above are suitable for producing RNAi. RNAi is synthesized either in vivo or in vitro. In some embodiments, endogenous RNA polymerase of the cell may mediate transcription in vivo, or cloned RNA polymerase can be used for transcription in vivo or in vitro. In other embodiments, the RNAi is provided transcription from a transgene in vivo or an expression construct. In some embodiments, the RNA strands are polyadenylated; in other embodiments, the RNA strands are capable of being translated into a polypeptide by a cell's translational apparatus. In still other embodiments, the RNA is chemically or enzymatically synthesized by manual or automated reactions. In further embodiments, the RNA is synthesized by a cellular RNA polymerase or a bacteriophage RNA polymerase (e.g., T3, T7, SP6, and the like). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. In some embodiments, the RNA is dried for storage or dissolved in an aqueous solution. In other embodiments, the solution contains buffers or salts to promote annealing, and/or stabilization of the duplex strands.

In some embodiments, the RNAi is transcribed from the vectors as two separate stands. In other embodiments, the two strands of DNA used to form dsRNAi may belong to the same or two different duplexes in which they each form with a DNA strand of at least partially complementary sequence. When the RNAi is thus-produced, the DNA sequence to be transcribed is flanked by two promoters, one controlling the transcription of one of the strands, and the other that of the complementary strand. These two promoters may be identical or different. In some embodiments, a DNA duplex provided at each end with a promoter sequence can directly generate RNAs of defined length, and which can join in pairs to form an RNAi. See, e.g., U.S. Pat. No. 5,795,715, incorporated herein by reference. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition; lower doses may also be useful for specific applications. Inhibition is sequence-specific in that nucleotide sequences corresponding to the duplex region of the RNA are targeted for genetic inhibition.

The RNAi, whether of synthetic or natural origin, is subject to rapid degradation by nucleases present in the sera of various animal species, particularly primates. Consequently procedures involving RNAi generally utilize baked glassware throughout, and all buffers are filtered, e.g., through a Nalgene 45 micron filter, for sterility. Pyrogen-free, double distilled water must be used for all solutions to minimize any possibility of endotoxin contamination.

Accordingly, in some embodiments, the present invention provides isolated RNA molecules (double-stranded or single-stranded) that are complementary to the JY-1 sequences of the present invention. It is contemplated that such molecules will find use in elucidating the function of JY-1 in early embryos. In particular, the RNAi probes of the present invention can be introduced into early embryos (e.g., one cell or two cell embryos) by microinjection or other suitable means so that the level of JY-1 in the embryos is reduced. The effect on development of the embryo can then be ascertained. Any RNAi can be used in the methods of the present invention, provided that it has sufficient homology to the targeted gene to mediate RNAi. RNA containing a nucleotide sequences identical to a portion of the target gene are preferred for inhibition. RNA sequences with insertions, deletions, and single point mutations relative to the target sequence have also been found to be effective for inhibition. Thus, sequence identity may optimized by sequence comparison and alignment algorithms known in the art (see Gribskov and Devereux, Sequence Analysis Primer, Stockton Press, 1991, and references cited therein, herein incorporated by reference) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases.

There is no upper limit on the length of the dsRNA that can be used. For example, the RNAi can range from about 21 base pairs (bp) of the gene to the full length of the gene or more. In one embodiment, the RNAi used in the methods of the present invention is about 1000 bp in length. In another embodiment, the RNAi is about 500 bp in length. In yet another embodiment, the RNAi is about 22 bp in length. In some preferred embodiments, the sequences that mediate RNAi are from about 21 to about 23 nucleotides. That is, the isolated RNAs of the present invention mediate degradation of JY-1 mRNA.

RNA of 21-23 and 20-25 nucleotides of the present invention need be sufficiently similar to natural RNA that it has the ability to mediate (mediates) RNAi for JY-1 message. In one embodiment, the present invention relates to RNA molecules of about 20 to about 25 nucleotides that direct cleavage of specific mRNA to which their sequence corresponds. It is not necessary that there be perfect correspondence of the sequences, but the correspondence must be sufficient to enable the RNA to direct RNAi cleavage of the target mRNA. In a particular embodiment, the 20-25 nt RNA molecules of the present invention comprise a 3′ hydroxyl group.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

In some embodiments, the dsDNA compositions of the present invention are provided as pharmaceutical compositions. Such formulations are described in more detail below.

### VIII. Pharmaceutical Compositions Containing JY-1 Nucleic Acid, Peptides, and Analogs.

The present invention further provides pharmaceutical compositions which may comprise all or portions of JY-1 polynucleotide sequences, JY-1 polypeptides, inhibitors or antagonists of JY-1 bioactivity, including JY-1 peptides and antibodies, alone or in combination with at least one other agent, such as a stabilizing compound, and may be administered in any sterile, biocompatible pharmaceutical carrier, including, but not limited to, saline, buffered saline, dextrose, water and adjuvants.

In some preferred embodiments, the proteins and peptides of the present invention find use in the vaccination of a subject. Accordingly, the peptides or proteins are preferably combined with an adjuvant and administered to a subject. It is contemplated that this procedure will allow determination of the function of JY-1 in ovarian related processes such as follicular development, ovulation and or subsequent fertility.

The peptides and proteins of the present invention also find use in treating diseases or altering physiological states. Peptides can be administered to the patient intravenously in a pharmaceutically acceptable carrier such as physiological saline. Standard methods for intracellular delivery of peptides can be used (e.g., delivery via liposome). Such methods are well known to those of ordinary skill in the art. The formulations of this invention are useful for parenteral administration, such as intravenous, subcutaneous, intramuscular, and intraperitoneal. Therapeutic administration of a polypeptide intracellularly can also be accomplished using gene therapy as described above.

As is well known in the medical arts, dosages for any one patient depends upon many factors, including the patient's size, body surface area, age, the particular compound to be administered, sex, time and route of administration, general health, and interaction with other drugs being concurrently administered.

Accordingly, in some embodiments of the present invention, JY-1 nucleotide and JY-1 amino acid sequences can be administered to a patient alone, or in combination with other nucleotide sequences, drugs or hormones or in pharmaceutical compositions where it is mixed with excipient(s) or other pharmaceutically acceptable carriers. In one embodiment of the present invention, the pharmaceutically acceptable carrier is pharmaceutically inert. In another embodiment of the present invention, JY-1 polynucleotide sequences or JY-1 amino acid sequences may be administered alone to individuals subject to or suffering from a disease.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries that facilitate processing of the active compounds into preparations that can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (i.e., dosage).

Pharmaceutical preparations that can be used orally include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising a compound of the invention formulated in a pharmaceutical acceptable carrier may be prepared, placed in an appropriate container, and labeled for treatment of an indicated condition. For polynucleotide or amino acid sequences of JY-1, conditions indicated on the label may include treatment of condition related to apoptosis.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

For any compound used in the method of the invention, the therapeutically effective dose can be estimated initially from cell culture assays. Then, preferably, dosage can be formulated in animal models (particularly murine models) to achieve a desirable circulating concentration range that adjusts JY-1 levels.

A therapeutically effective dose refers to that amount of JY-1 that ameliorates symptoms of the disease state. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature (See, U.S. Pat. No. 4,657,760; 5,206,344; or 5,225,212, all of which are herein incorporated by reference). Those skilled in the art will employ different formulations for JY-1 than for the inhibitors of JY-1. Administration to the bone marrow may necessitate delivery in a manner different from intravenous injections.

Results of the present studies demonstrate that the bovine JY-1 gene encodes for a novel oocyte-specific protein with important regulatory roles in granulosa cell function and early embryonic development and suggest that evolution of a functional JY-1 gene may be species specific. Multiple oocyte-specific genes have been described in mice that directly regulate either folliculogenesis or early embryonic development (Matzuk et al. (2002) Science 296, 2178-2180, herein incorporated by reference). However, the inventors contemplate that demonstration of a functional role for a single oocyte-specific gene (JY-1) in regulation of function of ovarian granulosa cells and early embryogenesis is unprecedented.

Identification of JY-1 like sequences corresponding to a small 3′ portion of the ORF and (or) the 3′UTR portion of the bovine JY-1 cDNA on syntenic chromosomes to bovine chr-29 in human, chimpanzee, mouse, rat and dog (Itoh et al. (2005) Genomics 85, 413-424; Band et al. (2000) Genome Res 10, 1359-1368; Cheng et al. (2005) Nature 437, 88-93; and Kirkness et al. (2003) Science 301, 1898-1903, all of which are herein incorporated by reference) raises the possibility that the JY-1 locus is conserved in multiple vertebrate species. The sequence identity of the human EST (from erythroid precursor cells) with JY-1 like sequence on human chr-11 (syntenic to bovine chr-29) suggests that a mRNA transcript may be transcribed from the above locus. However, the syntenic loci do not encode for the complete JY-1 gene and lack sequences corresponding to exons 1 and 2 and thus a significant portion of the protein coding region. It appears unlikely based on extensive sequence analysis that above loci in other species including humans encode for a protein of similar identity to bovine JY-1. Therefore, evolution of the novel oocyte-specific JY-1 protein is most likely species specific. However, the presence of a functional orthologs(s) performing similar roles as JY-1 in other mammalian species cannot be ruled out. Further, the significance of the conserved JY-1 3′UTR in multiple species is not known, but based on accumulating evidence for important regulatory roles of noncoding RNAs (Prasanth et al. (2007) Genes Dev 21, 11-42, herein incorporated by reference), a functional role for the observed JY-1 like sequence in the genome of other species cannot be discounted.

To suit the diverse reproductive functions in mammals, certain genes or gene families may have evolved by selection pressure during the course of evolution. For example, the trophoblast cell derived pregnancy recognition factor interferon-tau is produced in mammals within the Ruminantia suborder (e.g. cattle, sheep, goats), but not in unrelated species (Roberts et al. (2003) Reprod Suppl 61, 239-251, herein incorporated by reference). Recent identification of the trophoblast kunitz domain protein (TKDP) gene family specifically expressed in ruminants (Chakrabarty et al. (2006) Gene 373, 35-43, herein incorporated by reference) further supports the concept that certain genes in the reproductive system may have evolved in a species specific fashion and been selected for specialized functions. The evolution of above genes in ruminants may be attributed to clear species specific differences in the trophoblast and the type of placentation mediating maternal-fetal communication (Carter et al. (2004) Reprod Biol Endocrinol 2, 46, herein incorporated by reference). Species specific attributes of oocyte function in general are not well understood, but bovine versus mouse oocytes do differ in their requirement for cumulus cell expansion and ability to promote glucose uptake by such cels (Zuelke et al. (1992) Endocrinology 131, 2690-2696; Sutton et al. (2003) Reproduction 126, 27-34; Eppig et al. (1993) Dev Biol 158, 400-409; Sugiura et al. (2005) Dev Biol 279, 20-30; and Ralph et al. (1995) Mol Reprod Dev 42, 248-253, all of which are herein incorporated by reference). Studies in closely related species (e.g. sheep and goats) will be necessary to further determine the specificity in structure and function of the JY-1 gene.

Results support an important role for JY-1 in early embryonic development in cattle. The inventors demonstrate herein that JY-1 mRNA was dynamically regulated during the window from meiotic maturation through embryonic genome activation. Results of siRNA mediated gene knockdown experiments in two different in vitro models of early embryogenesis support a requirement of JY-1 for development to the blastocyst stage in cattle. Results also suggest that the maternal JY-1 mRNA is translated during bovine early embryogenesis because siRNA mediated mRNA knockdown prevented the accumulation of JY-1 protein in early embryos. The inventors' results further show that JY-1 is required during the early embryonic stages prior to embryonic genome activation because JY-1 siRNA injection reduced development of embryos to the 8-16 cell stages by ˜40% and merely 25% of injected embryos reaching the 8-16 cell stage developed into blastocysts. Gene targeting approaches have demonstrated the role of oocyte-specific MATER, ZAR1 and NPM2 genes for early embryo development in mice (Roy et al. (2006) Reproduction 131, 207-219, herein incorporated by reference). Embryonic genome activation occurs much later in domestic ruminants (e.g. 8-16 cell stages in cattle, sheep) compared to the mouse (at 2-cell stage), thus additional maternal effect genes may be required to promote early embryogenesis in such species. While MATER and ZAR1 expression in bovine oocytes/embryos has been reported (Pennetier et al. (2006) BMC Dev Biol 6, 26; Pennetier et al. (2004) Biol Reprod 71, 1359-1366, all of which are herein incorporated by reference), experimental evidence is lacking to support the requirement of above genes for bovine early embryogenesis. To the inventors knowledge, JY-1 is the first known oocyte-specific maternal factor demonstrated to govern early embryonic development in non-murine species.

Oocyte regulation of folliculogenesis and phenotype/function of ovarian somatic (cumulus and granulosa cells) were established (Matzuk et al. (2002) Science 296, 2178-2180; Eppig et al. (2001) Reproduction 122, 829-838, all of which are herein incorporated by reference). Results of the studies described herein, demonstrate pronounced effects of rJY-1 protein on granulosa cell phenotype in a manner mimicking preovulatory events characteristic of the luteinization process. Biological actions of JY-1 on bovine granulosa cells are novel and do not mimic the reported effects of the well known oocyte-specific growth factors GDF9 and BMP 15 on bovine granulosa cell function (McNatty et al. (2005) Reproduction 129, 481-487; Spicer et al. (2006) J Endocrinol 189, 329-339, herein incorporated by reference). In vivo, the preovulatory gonadotropin (LH) surge results in decreased E and increased P levels in the follicular fluid of bovine preovulatory follicles (Li et al. (2007) J Endocrinol 192, 473-483, herein incorporated by reference) and preovulatory granulosa cells exit from the cell cycle and are transformed into non-dividing terminally differentiated luteal cells capable of producing high levels of P (Richards, et al. (2001) J Soc Gynecol Investig 8, S21-23; Quirk et al. (2004) J Anim Sci 82 E-Suppl, E40-52, herein incorporated by reference). The inventors observed that in culture studies, an increase in P was accompanied by a suppression of the FSH stimulated increase in granulosa cell numbers and E production, mimicking the in vivo preovulatory follicular environment. Thus the inventors contemplate the use of rJY-1 for a variety of applications for inducing preovulatory follicular development.

The following references are all herein incorporated in their entirety: Matzuk, et al., Science 2002, 296:2178-2180; Eppig, Reproduction 2001, 122:829-838; Eppig, et al., Proc Natl Acad Sci U.S.A 2002, 99:2890-2894; Li, et al., Biol Reprod 2000, 63: 839-845; Telford, et al., Mol Reprod Dev 1990, 26:90-100; Yao, et al., Physiol Genomics 2004, 19: 84-92; Lin, et al., Semin Reprod Med 2005, 23:201-212; Hanrahan, et al., Biol Reprod 2004, 70:900-909; Moore, et al., Trends Endocrinol Metab 2004, 15:356-361; Galloway, et al., Mol Cell Endocrinol 2002, 191:15-18; Galloway, et al., Nat Genet. 2000, 25:279-283; Yan, et al., Mol Endocrinol 2001, 15:854-866; Cassar, et al., Domest Anim Endocrinol 2002, 22:179-187; Dow, et al., Biol Reprod 2002, 66:1413-1421; Li, et al., Reproduction 2004, 128:555-564; Bakke, et al., Biol Reprod 2004, 71:605-612; Fortune, Anim Reprod Sci 2003, 78:135-163; Gutierrez, et al., Biol Reprod 1997, 56:608-616; Kobayashi, et al., Endocrinology 2004, 145:5373-5383; Jimenez-Krassel, et al., Endocrinology 2003, 144:1876-1886; Bettegowda, et al., Mol Reprod Dev 2006, 73:267-278; Schnorf, et al., Exp Cell Res 1994, 210:260-267; van Soom, et al., Mol Reprod Dev 1997, 47:47-56; De La Fuente, et al., Biol Reprod 1998, 58:952-962; Nielsen, et al., Protein Eng 1999, 12:3-9; Julenius, et al., Glycobiology 2005, 15:153-164; McGuffin, et al., Bioinformatics 2000, 16:404-405; Marchler-Bauer, et al., Nucleic Acids Res 2003, 31:383-387; Marchler-Bauer, et al., Nucleic Acids Res 2002, 30:281-283; Bateman, et al., Nucleic Acids Res 2000, 28:263-266; Altschul, et al., Nucleic Acids Res 1997, 25:3389-3402; Yan, et al., Biol Reprod 2006, 74:999-1006; Liang, et al., Development 1997, 124:4939-4947; Andreu-Vieyra, et al., Trends Endocrinol Metab 2006, 17:136-143; Itoh, et al., Genomics 2005, 85: 413-424; Band, et al., Genome Res 2000, 10: 1359-1368; Cheng, et al., Nature 2005, 437: 88-93; Kirkness, et al., Science 2003, 301:898-1903; Demmers, et al., Reproduction 2001, 121:41-49; Chakrabarty, et al., Gene 2006v373:35-43; Chakrabarty, et al., J Mol Evol 2006, 63:274-282; Carter, et al., Reprod Biol Endocrinol 2004, 2:46; Tong, et al., Nat Genet. 2000, 26:267-268; Wu, et al., Nat Genet. 2003, 33:187-191; Burns, et al., Science 2003, 300:633-636; Pennetier, et al., BMC Dev Biol 2006, 6:26; Pennetier, et al., Biol Reprod 2004, 71:1359-1366; McNatty, et al., Reproduction 2005, 129:481-487; Spicer, et al., J Endocrinol 2006, 189:329-339; Li, et al., J Endocrinol 2007, 192:473-483; Richards, et al., J Soc Gynecol Investig 2001, 8:S21-23; and Quirk, et al., J Anim Sci 2004, 82 E-Suppl: E40-52.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); U (units), mU (milliunits); min. (minutes); sec. (seconds); % (percent); kb (kilobase); bp (base pair); PCR (polymerase chain reaction); BSA (bovine serum albumin); Fisher (Fisher Scientific, Pittsburgh, Pa.); Sigma (Sigma Chemical Co., St. Louis, Mo.); Promega (Promega Corp., Madison, Wis.); Perkin-Elmer (Perkin-Elmer/Applied Biosystems, Foster City, Calif.); Boehringer Mannheim (Boehringer Mannheim, Corp., Indianapolis, Ind.); Clonetech (Clonetech, Palo Alto, Calif.); Qiagen (Qiagen, Santa Clarita, Calif.); Stratagene (Stratagene Inc., La Jolla, Calif.); National Biosciences (National Biosciences Inc, Plymouth Minn.), NEB (New England Biolabs, Beverly, Mass.), wt (wild-type); Ab (antibody); GV (germinal-vesicle); GVO (germinal vesicle oocytes); BS (bull serum); GS (granulosa cells); L (liver); A (adrenal gland); chr (chromosome); E (estradiol); P (progesterone); rJY-1 (recombinant JY-1); FSH (follicle-stimulating hormone); CPE (cytoplasmic polyadenylation elements); IVF (In-Vitro Fertilization); ET (Embryo Transfer), FET (Frozen Embryo Transfer); IVF-ET (In-Vitro Fertilization and Embryo Transfer); and BSA (Bovine Serum Albumin).

### Example I

The following descriptions are provided as exemplary Materials and Methods.

Northern Blot Analysis. Northern blotting was performed as previously described (Bakke, et al., (2004) Biol. Reprod., 71:605-612; herein incorporated by reference). In brief, an exemplary Northern Blot Procedure was done as follows: Total RNA was isolated from bovine tissues using Trizol reagent (Invitrogen Life Technologies, Carlsbad Calif.) followed by poly (A)+ RNA isolation using PolyATtract mRNA Isolation System (Promega, Madison Wis.). Total RNA equivalent to 300 GV oocytes was isolated using RNAqueous micro kit (Ambion, Austin Tex.), followed by DNAse digestion as per manufacturer's instructions. Northern blotting was performed with ³²P-labeled JY-1 cDNA according to the inventor's previously published procedures [Cassar, et al., Domest Anim Endocrinol 2002; 22:179-187, herein incorporated by reference]. The blots were stripped and re-probed with ³²P-labeled bovine RPL-19 (Ribosomal protein L19) cDNA or actin as a positive control.

Bovine Fetal Ovary cDNA Library Construction. Fetal ovary RNA was collected at day 210 of gestation was isolated as described above. A bovine fetal ovary cDNA library was prepared using a ZAP-cDNA® Synthesis Kit in a Lambda ZAP® II vector (Stratagene, La Jolla, Calif.) according to manufacturer's instructions (see, Stratagene's Instruction Manuals, herein incorporated by reference). Positive plaques were screened by well known methods of hybridization with ³²P-labeled JY-1 cDNA, followed by plaque purification, phagemid DNA purified with the DNA subjected to fluorescent dye terminator sequencing.

Western Blotting. Recombinant JY-1 protein (rJY-1, predicted mature protein without signal peptide) was expressed in BL21 E. coli and purified commercially by C & P Biotech Corp. using the pET 15b vector. Polyclonal antiserum was generated in rabbits against rJY-1 protein by ABR-Affinity BioReagents™, Incorporated. Western blotting was performed using the inventors' established protocols (Bakke, et al. (2004) Biol Reprod 71:605-612; all of which are herein incorporated by reference). In brief, recombinant JY-1 protein (rJY-1, predicted mature protein without signal peptide) was expressed in BL21 E. coli and purified commercially by C & P Biotech Corp., Golden, Colo., using the pET15b vector. Polyclonal antiserum was generated in rabbits against rJY-1 protein by Affinity Bioreagents. Western blotting was performed using the inventors protocols [Li, et al., Reproduction 2004; 128:555-564, herein incorporated by reference]. Protein lysates of 150 GV oocytes per lane were used in each blot. Approximately 10 μg protein from bull serum, granulosa cells, liver and adrenal tissues were also included in the analysis. As a positive control approximately 12.5 ng of rJY-1 protein/lane was included. Blots were treated with a 1:1000 dilution (v/v) of JY-1 antiserum or a 1:1000 dilution (v/v) preimmune serum or a 1:1000 dilution (v/v) of JY-1 antiserum preincubated with approximately 5 μg rJY-1 protein (immunogen) overnight at 4° C. prior to incubation with blots. Goat anti-rabbit IgG (Amersham Biosciences, Bucks, UK) was used as a secondary antibody at 1:2500 (v/v) dilution. Immunoreactive proteins were visualized using a chemiluminescent horseradish peroxidase detection system (Genotech, St. Louis Mo.). Membranes were stripped using Restore Western Blot Stripping Buffer (Pierce, Rockford Ill.) and probed for actin as described previously [Li, et al., Reproduction 2004; 128:555-564, herein incorporated by reference].

In Situ Hybridization. In situ hybridization was performed as described Bakke, et al. (2002) Biol Reprod 66:1627-1634; all of which are herein incorporated by reference. In brief, approximately 14 μm sections of ovarian tissues were cut on a cryostat and mounted onto positively charged slides (Fisher Scientific, Chicago Ill.). Prior to hybridization, sections are prewarmed to room temperature for 10 min, fixed in 3.7% formaldehyde in PBS for 5 min, rinsed twice in 2×SSC for 2 min each, incubated in 0.25% acetic anhydride in 0.1 M triethanolamine-HCl (pH 8.0) for 10 min, dehydrated in increasing concentrations of ethanol (70, 80, 95 and 100%) for 2 min each, delipidated in absolute chloroform for 5 min, rinsed in 100% and 95% ethanol for 2 min each and then air dried for 1 hour. Prior to hybridization, labeled probes are diluted in hybridization buffer to a concentration of 1.0×10⁶ cpm/ml. Hybridization buffer includes 50% formamide, 0.3 M sodium chloride, 10 mM Tris (pH 8), 1 mM EDTA, 1×Denhardt's, 50 mM dithiothreitol (DTT), 0.5 mg/ml yeast tRNA and 10% dextran sulfate. Hybridizations for JY-1 mRNA were carried out on approximately 15 serial sections using antisense and sense (negative control) ³⁵S labeled cRNA probes generated from the 450 bp JY-1 cDNA. For each tissue sample (n=3 samples), approximately 10 sections were hybridized with the antisense probe and 5 sections with the sense probe (every other adjacent section). Hybridizations were performed by adding 60 μl diluted probe/slide and then incubating in a humidified 55° C. oven for 16 hour. After hybridization, slides are washed twice by shaking in 2×SSC for 15 min at room temperature and treated with RNase-A (50 μg/ml) in 2×SSC for 1 h at 37° C. Slides are then washed at 55° C. in 2×SSC containing 0.1% β-mercaptoethanol (B3ME) for 15 min, 1×SSC/0.1% B3ME for 15 min, 1×SSC/50% formamide/0.1% BME for 30 min, and twice in 0.1×SSC/0.1% BME for 15 min at 65° C. The slides are then dehydrated in increasing ethanol concentrations (60, 80, 95 and 100%); air dried for 1 h and dipped in 50% NTB-2 emulsion (Eastman Kodak, Rochester N.Y.). Slides are exposed to autoradiographic emulsion for varying lengths of time depending on mRNA abundance and signal intensity (generally 3-7 days for JY-1) at 4° C. and then developed followed by counterstaining with hematoxylin and eosin. Digital bright and dark-field images are then acquired on a Leica research microscope equipped with SPOT Model #1.1.0 camera and Version 3.2.4 software. Intraovarian localization of mRNAs to specific cell types and follicle classes were determined as described herein.

Immunohistochemistry. Polyclonal antiserum was generated against a 20-amino acid synthetic peptide corresponding to a portion of the carboxy-terminus of the predicted amino acid sequence of bovine JY-1 (C55-A74). Peptide synthesis, conjugation to keyhole limpet hemocyanin, immunization, and immunoaffinity purification was conducted commercially by Bethyl Laboratories. Immunocytochemical localization of JY-1 protein (n=3 samples) was performed as described by the inventors [Li, et al., Reproduction 2004 128:555-564; Bakke, et al., Biol Reprod 2004 71:605-612, each of which are herein incorporated by reference]. In brief, ovarian sections were treated with approximately a 1/300 dilution (5 μg/ml) of affinity purified anti JY-1 IgG or 5 μg/ml preimmune IgG or 5 μg/ml affinity purified anti JY-1 IgG preincubated with 100 fold excess peptide (immunogen) overnight at 4° C. prior to incubation with sections. Previously published criteria were used to classify bovine follicles into specific developmental stages including the counting the number of layers and determining the morphology of granulosa cells [Fortune, Anim Reprod Sci 2003 78:135-163, herein incorporated by reference].

Effect of rJY-1 on Granulosa Cell Function. Serum-free long-term granulosa cell (GC) culture was performed as described previously (Gutierrez et al. (1997) Biology Reprod 56:608-616; Sen et al. (2007) Endocrinology 148:4400-4410; herein incorporated by reference). In brief, serum-free long-term granulosa cell (GC) culture was performed as described previously [Gutierrez, et al., 1997 Biol Reprod 56:608-616, herein incorporated by reference], with slight modifications. Briefly, GC from 3-5 mm follicles collected at random stages of the estrous cycle were pooled in MEM a culture media supplemented with sodium bicarbonate (10 mM), HEPES (20 mM), antibiotics (100 U/ml penicillin and 0.1 mg/ml streptomycin), fungizone-amphotericin B (250 μg/ml), non-essential amino acids (1.1 mM), bovine insulin (10 ng/ml), long R3-IGF-I (1 ng/ml), sodium selenite (4 ng/ml), apo-transferin (5 μg/ml) and androstenedione (10⁻⁷ M). The cells were washed, re-suspended in media and cell number and viability estimated via trypan blue exclusion. Cells (1×10⁵ viable cells/well) were cultured in media containing 0 or 25 ng/ml FSH at 37° C. in a humidified atmosphere (5% CO₂ and 95% air) for 7 days, with 75% medium replaced every 48 h with fresh medium. The FSH (NIDDK-oFSH-20) was supplied from Harbor-UCLA Research and Education Institute (Los Angeles Calif.). The rJY-1 protein at 0, 0.1, 0.5, 1 and 10 ng/ml final concentration per culture was supplemented throughout the culture with fresh protein added every 48 h at medium replacement (n=3-7 replicates/treatment). On day 7 of culture period, media were collected and stored at −20° C. for subsequent measurement of progesterone (P) and estradiol (E) by RIA [Kobayashi, et al., 2004 Endocrinology 145:5373-5383; Jimenez-Krassel, et al., 2003 Endocrinology 144:1876-1886, herein incorporated by reference]. The cells were washed 2× with Dulbecco's PBS, trypsinized and cell number determined using a Coulter Counter Particle Z1 [Jimenez-Krassel, et al., 2003 Endocrinology 144:1876-1886, herein incorporated by reference] (Beckman Coulter, Inc., Fullerton Calif.). Each treatment was administered in triplicate and P and E levels were normalized to 30,000 cells.

Quantification of JY-1 mRNA in Oocytes and Early Embryos. Oocyte recovary, in vitro maturation, in vitro fertilization, embryo culture and quantification of mRNA by real-time PCR were performed as described previously Bettegowda, (2006) Mol. Reprod. Dev. 73:267-278, herein incorporated by reference. In brief, oocyte recovary, in vitro maturation, in vitro fertilization and embryo culture were performed according to the inventors previously published procedures [Bettegowda, et al., Mol Reprod Dev 2006; 73:267-278, herein incorporated by reference]. Oocyte and embryo sample collection (germinal vesicle (GV) and metaphase (MII) stage (oocytes), pronucleus (PN), 2-cell, 4-cell, 8-cell, 16-cell, morula and blastocyst stage (embryos); n=5 pools of 10 each), RNA extraction, cDNA synthesis (with dT primers or random hexamers) and JY-1 mRNA quantification by real-time PCR were also performed using the inventors' procedures reported previously [Bettegowda, et al., Mol Reprod Dev 2006; 73:267-278, herein incorporated by reference]. Two hundred fifty femtograms of polyadenylated GFP mRNA were added to each sample prior to RNA extraction. Oligo dT(18) primers were used to reverse transcribe polyadenylated transcripts, whereas random hexamers were used to reverse transcribe total transcripts. The forward and reverse real-time PCR primers for JY-1 were 5′-TTGGAACTTCCATGGACGACC-3′ (SEQ ID NO: 30) and 5′-ATTTGCTGGTGATCCCAAGAG-3′ (SEQ ID NO: 31), respectively.

Preparing cDNA for RT-PCR analysis. Samples of fetal ovary tissue, (an enriched source of oocytes), fetal testis, spleen heart, muscle, lung, adult testes, uterus, thymus, kidney, liver, adrenal, hypothalamus, cortex, gut, pituitary, bone marrow (sternum and leg), leukocytes and metaphase II and germinal vesicle stage oocytes were subjected to RNA isolation using TRIzol reagent (Invitrogen). After RNA isolation and confirmation of integrity, 1 μg total RNA was treated with RNase free DNase I (Invitrogen) and a fraction (91 ng) utilized in first strand cDNA synthesis with Superscript II reverse transcriptase (RT) according to manufacturer's instructions. Synthesized cDNA was then used as template for amplification of cDNA encoding for JY-1 using specific primers and standard PCR procedures. PCR products were visualized following electrophoresis on a 1.2% agarose gel. Reverse transcriptase (RT) minus and no cDNA were controls utilized for each primer set/tissue to confirm absence of contaminating genomic DNA or cDNA carryover. Amplification of a 360 bp cDNA encoding for bovine RPL19 was used as a positive control to confirm that cDNA synthesis was successful.

Synthesis and Validation of siRNA Species by Microinjection. To determine the effects of JY-1 knockdown on blastocyst development, microinjection experiments were performed in two different in vitro models of embryo development: parthenogenesis and IVF (n=4-5 replicates/treatment). In brief, a publicly available siRNA design algorithm (siRNA target finder, Ambion) was used to design siRNA species targeting the ORF of JY-1 mRNA. Candidate siRNA species were interrogated using a BLAST homology program to rule out homology to any other known genes in the bovine EST and genomic database. Two distinct JY-1 siRNA species were chosen, synthesized and then dissolved in nuclease free water using the Silencer siRNA construction kit (Ambion) following manufacturer's instruction. The sense (S) and antisense (AS) oligonucleotide template sequence were as follows: JY-1 siRNA species-1 (S-5′-AATGGGTGGTCTGTGAAGAACCCTGTCTC-3′) (SEQ ID NO:32); AS: 5′-AAGTTCTTCACAGACCACCCACCTGTCTC-3′) (SEQ ID NO:33); JY-1 siRNA species 2 (S: 5′-AACAATCAGGGAGCTGAGTATCCTGTCTC-3′ (SEQ ID NO:34); AS 5′-AAATACTCAGCTCCCTGATTGCCTGTCTC-3′) (SEQ ID NO:35).

Microinjection was performed using an inverted Nikon microscope (Nikon Inc.) equipped with Narishige micromanipulators (Narishige International USA Inc., New York, United States). In brief, an ICSI Micropipet (Humagen Fertility Diagnostics, Charlottesville, Va., United States) was filled with siRNA or water, inserted into oocyte cytoplasm followed by brief suction of the oocyte cytoplasm to ensure the puncture of the cytoplasmic membrane before injection of approximately 20 picoliter. The average microinjection volume per injection (20 picoliter) was determined by bubble pressure measurement [Schnorf, et al., Exp Cell Res 1994; 210:260-267, herein incorporated by reference]. Success rates of microinjection procedure were validated by injection of neutral Texas-red dextran dye-3000 MW (Invitrogen) into denuded MII oocytes followed by fluorescence microscopy (Nikon Inc., USA). Each individual siRNA was validated for efficiency in RNA knockdown following microinjection of MII eggs, parthenogenetic activation and collection of embryo samples at the 4-cell stage (41-43 h after oocyte activation) for quantification of JY-1 mRNA abundance by real-time PCR. Controls were sham injected with a similar volume of water. Two of the validated JY-1 siRNA species with >90% efficiency in targeting JY-1 mRNA were combined at a final concentration of 25 μM and are denoted as JY-1 cocktail siRNA. As a control for siRNA injection, negative control siRNA (nonspecific; Ambion universal control #1) was also tested as described above except that blastocyst development on day 7 post injection was used as the endpoint. Quality of blastocysts derived from uninjected controls versus negative control siRNA injection was also confirmed by counting total cell numbers (by Hoechst nuclear staining) [van Soom, et al., Mol Reprod Dev 1997; 47:47-56; De La Fuente, et al., Biol Reprod 1998; 58:952-962, herein incorporated by reference]. Reduction of JY-1 protein in JY-1 siRNA injected parthenogenetic embryos collected at the 8-16 cell stages (72 h after parthenogenetic activation) was determined by Western blot analysis. Uninjected 8-16 cells embryos generated at the same time as microinjection experiments were used as the control. Protein lysates from 185 embryos per treatment (n=3 samples of 50 embryos each, and n=1 sample of 35 embryos each) were loaded per lane. To determine the effects of JY-1 knockdown on blastocyst development, microinjection experiments were performed in two different in vitro models of embryo development: parthenogenesis and IVF. In each experiment (n=4-5 replicates), groups of approximately 25-30 denuded MII oocytes or denuded presumptive one cell embryos derived from IVF were randomly assigned to one of the following treatments: 1) JY-1 cocktail siRNA (25 μM), 2) negative control siRNA-1 (25 μM), 3) sham water injection or 4) uninjected controls. After microinjection, groups of activated oocytes or IVF embryos were cultured in 75 μl drops of KSOM supplemented with BSA (3 mg/ml). Cleaved embryos were separated and cultured in fresh drops of KSOM supplemented with 10% FBS and BSA (3 mg/ml). Cleavage rate was determined on day 3, and blastocyst rate (total no. of blastocysts/total cleaved embryos) assessed on day seven.

Parthenogenetic Activation. Denuded MII oocytes were activated in 5 μM ionomycin (Calbiochem, San Diego Calif.) in HEPES buffered-Hamster embryo culture medium (HECM) for 4 min, followed by 4 h incubation in 2 mM 6-dimethylaminopurine (6-DMAP) (Sigma) in KSOM medium supplemented with 3 mg/ml BSA. After incubation, the oocytes were washed in drops of HH medium for 4 min and cultured in KSOM medium supplemented with 3 mg/ml BSA.

Genomic Southern Blot Analysis. In brief, genomic DNA was extracted from cow, sheep, pig, mouse and chicken (liver tissue) and whole rainbow trout and zebrafish, followed by residual RNA digestion and DNA precipitation. RNAse treated human genomic DNA (source: liver) was purchased from a commercial vendor (Biochain Institute Inc., Hayward Calif.). Southern blot was prepared by digesting approximately 15 μg of genomic DNA per sample with EcoRI for 30 hr, electrophoretically separated and transferred to a Zeta-probe membrane (Bio-Rad, Hercules Calif.). The membrane was hybridized at 62° C. in PerfectHyb solution (Sigma-Aldrich, St. Louis Mo.) with ³²P-labeled JY-1 cDNA (450 bp, region corresponding to 5′UTR, ORF and a portion of 3′UTR). The membrane was washed twice at 60° C. in 0.5×SSC/0.1% SDS for 15 minutes and subjected to autoradiography.

Genomic Library Screening and Bioinformatics analysis. An exemplary JY-1 gene structure was determined by screening a EMBL3 SP6/T7 bovine genomic library (Clontech 1 Laboratories, Inc., Palo Alto Calif.) using standard procedures (Sambrook) for identifying, isolating, and sequencing JY-1 bovine gene fragments. Positive plaques were identified by replica plating onto nylon membranes (Hybond-N, Amersham) where the phage DNA was denatured, neutralized and affixed to membranes by UV crosslinking. Membranes were hybridized in PerfectHyb solution (Sigma) with a (α-³²P) dCTP radiolabeled 5′ cDNA probe corresponding to nucleotides 26-320 of the bovine JY-1 cDNA. After hybridization, membranes were washed in 2×SSC/0.1% SDS at 65° C. for 30 min and then with 0.1% SSC/0.1% SDS at 65° C. for 15 min and were exposed to X-ray film. Positive plaques were isolated and confirmed by hybridization to JY-1 fragments and PCR analysis. The positive clones from these plaques were propagated in K802 Escherichia coli cells on Luria broth agar plates. The lambda phage DNA was purified for subcloning and sequencing using Wizard Lambda Preps DNA Purification System (Promega) and a lambda maxi kit as per the manufacturer's instructions (Qiagen). Phage DNA was digested with restriction endonucleases and subcloned into pBluescript SK (±) vector (Stratagene) for subsequent fluorescent dye terminator sequencing.

The gene structure for JY-1 was determined using restriction endonuclease digestion, agarose gel electrophoresis, Southern blot analysis and by sequencing of fragments of interest. Beginning with a partial JY-1 gene sequence, the complete gene structure and chromosomal location for the JY-1 gene was determined by searching the bovine genome database at NCBI. Genomic DNA databases at NCBI for chimpanzee, dog, mouse, rat, chicken, zebrafish and drosophila were then searched with the predicted amino acid and nucleotide sequence of the 1.5 kb bovine JY-1 cDNA. Likewise, the human EST database was searched with the nucleotide sequence of the 1.5 kb bovine JY-1 cDNA.

Cloning of Putative Human JY-1 cDNA. An exemplary human EST sequence derived from a Hembase library (erythroid precursor cells, GenBank accession: BU656412) showing identity to a small portion of the ORF present in the bovine JY-1 cDNA sequence was identified following a search of the human EST database at NCBI GenBank. Two pairs of PCR primers were designed based on the human EST sequence and a two step nested PCR was performed. cDNA was synthesized from total RNA from adult human ovary (Stratagene) and H9 human embryonic stem cells (generously provided by Dr. Jose Cibelli) as described previously [Kobayashi, et al., Endocrinology 2004; 145:5373-5383, herein incorporated by reference]. Synthesized cDNA was used as template for first round PCR amplification of cDNA encoding for putative human JY-1 using specific primers (F: 5′-AAATCTGTGTGGATAGCCTTATCAG-3′ (SEQ ID NO:36) and R: 5′-CCTGGTGACAAAGAGAACATACG-3′(SEQ ID NO:37) and standard PCR procedures [Kobayashi, et al., Endocrinology 2004; 145:5373-5383, herein incorporated by reference], with 3 mM magnesium chloride concentration. Negative controls reactions for cDNA synthesis incubated in the absence of reverse transcriptase enzyme were included to confirm absence of residual genomic DNA contamination. Nested PCR was performed with a second set of PCR primers (F: 5′-CCAGGCATGTTACTTATGAATAACTT-3′ (SEQ ID NO:38) and R: 5′-AGGGAGCTGAAGCTTGGAA-3′ (SEQ ID NO:39) using 1 μl of first round PCR amplification product from respective RT plus and RT minus reactions. Amplification of the housekeeping gene beta-actin (β-actin) with PCR primers (F: 5′-TCCTCCCTGGAGAAGAGCTA-3′ (SEQ ID NO:40) and R: 5′-AGTACTTGCGCTCAGGAGGA-3′(SEQ ID NO:41) spanning two introns was used as a positive control to verify cDNA synthesis and to confirm absence of genomic DNA contamination. Amplified cDNA were ligated into pCR 2.1 TOPO vector (Invitrogen) and plasmids containing cDNA of interest were subjected to fluorescent dye terminator sequencing.

Statistical Analysis. For real-time PCR experiments, differences in mRNA abundance were determined by one-way analysis of variance using the GLM procedure of SAS. For microinjection experiments, rates of embryo development to 8-16 cell and blastocyst stages were analyzed following arcsin transformation using the Mixed Linear Models procedure of SAS. Similarly, differences in P, E and cell numbers were determined by the Mixed Linear Models procedure of SAS. Mean comparisons were performed using Tukey's test. The dose response relationship between rJY-1 and P was determined by regression analysis. Differences of P<0.05 were considered significant.

Construction of the bovine oocyte cDNA library and EST sequence analysis procedures. A library was constructed based on SMART technology (BD Biosciences, Palo Alto, Calif.). Poly (A)+ RNA was isolated directly from a pool of 100 germinal vesicle stage and 100 metaphase II stage oocytes using an oligotex direct mRNA micro kit (Qiagen Ltd., Valencia, Calif.) following the manufacturer's instruction. The eluted mRNA was dried by speed vacuum and resuspended in 8 μl of water followed by addition of 1 μl each of a modified oligo-dT primer (1 μg/μl, 5′-GACTAGTTCTAGATCGCGAGCGG CCGCTTTTTTTTTTTTTTTTTTTTTTTT-3′) (SEQ ID NO:42) containing a NotI site and a SMART primer (1 μg/μl, 5′-AAGCAGTGGTAACAACGCAGAGTACGAATTCGTCGACGCGGG-3′) (SEQ ID NO:43) containing a SalI site. The mixture was incubated at 70° C. for 5 minutes and placed immediately on ice for 3 minutes. The volume was then adjusted to 20 μl with 4 μl of 5× first strand cDNA synthesis buffer, 2 μl of 0.1 M DTT, 1 μl of 10 mM dNTP, 2 μl of water and 1 μl of Superscript II reverse transcriptase (200 U/μl, Invitrogen) followed by incubation at 42° C. for 60 minutes in a thermal cycler. The synthesized cDNA (20 μl) was amplified by 35 cycles of PCR in a 200-μl reaction containing 1× Advantage 2 PCR buffer (BD Biosciences), 0.4 mM dNTP, 0.3 μM 5′-SMART PCR primer (5′-AAGCAGTGGTAACAACGCAGAGTAC-3′) (SEQ ID NO:44), 0.3 μM 3′-SMART PCR primer (5′-GACTAGTTCTAGATCGCGAGCGG-3′(SEQ ID NO:45)) and 4 μl of 50× Advantage 2 polymerase mix (BD Biosciences). The PCR product was extracted with phenol:chloroform:isoamyl alcohol (25:24:1), ethanol-precipitated and resuspended in 20 μl of water followed by digestion with SalI and NotI. The digested cDNAs were purified using a PCR purification kit (Qiagen) and fractionated through a Chroma Spin Columns 400 (BD Biosciences). The digested/size selected cDNAs were ligated to SalI/NotI pre-digested pSPORT1 vector (Invitrogen) and electroporated into DH10B competent cells.

The bovine oocyte cDNA library was plated onto 150-mm plates containing LB agar supplemented with 50 μg/ml ampicillin. Colonies were randomly picked from the plates, transferred to 96-well plates and cultured overnight. Plasmid DNA was isolated from the cultures using the QIAprep 96 Turbo miniprep system (Qiagen) according to the manufacturer's instructions. Sequencing reactions were performed at the Michigan State University Genomic Technologies Support Facility using a BigDye terminator sequencing kit (Perkin-Elmer/ABI, Palo Alto, Calif.) and analyzed on an ABI 3700 DNA analyzer. Sequence data were outputted to Geospiza software (Geospiza, Inc., Seattle Wash.) for quality assessment and vector sequence trimming. Final sequences were subjected to basic local alignment search tool (BLAST) searches against the GenBank non-redundant (nr) and expressed sequence tag (est) database using the BLASTN and BLASTX programs via netblasting. Clustering of the EST sequences was performed using an online clustering software, stackPACK™ v2.2 (bch.msu.edu/stackpack/index).

Northern blot analysis and further characterization of JY-1 cDNA. In order to characterize the size and number of JY-1 mRNA transcripts, several bovine tissues, including fetal ovary (an enriched source of oocytes), were collected, RNA was isolated and subjected to Northern analysis. Total RNA was isolated from bovine tissues using Trizol reagent (Invitrogen Life Technologies) followed by poly (A)+ RNA isolation using PolyATtract mRNA Isolation System (Promega). Approximately 3 μg of poly (A)+ RNA from each tissue was electrophoresed in a 1.2% agarose gel containing 2.2 M formaldehyde and transferred onto Zeta-probe membrane (Bio-Rad). The membrane was hybridized at 68° C. in PerfectHyb solution (Sigma) with ³²P-labeled PCR amplified JY-1 cDNA (455 bp). The membrane was washed twice in 2×SSC/0.1% SDS at room temperature and twice at 65° C. in 0.2×SSC/0.1% SDS and subjected to autoradiography. The blot was strip-washed and re-probed with bovine RPL19 as an RNA loading control.

### Example II

### Tissue Distribution and Characterization of JY-1 mRNA Transcripts

The experiments in this example demonstrated that JY-1 mRNA was detected in fetal ovary tissue but not in other tissues examined.

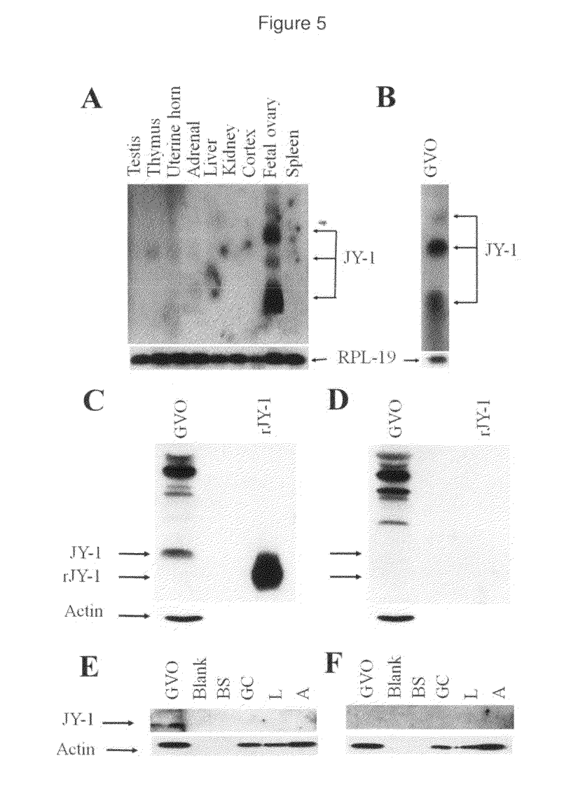

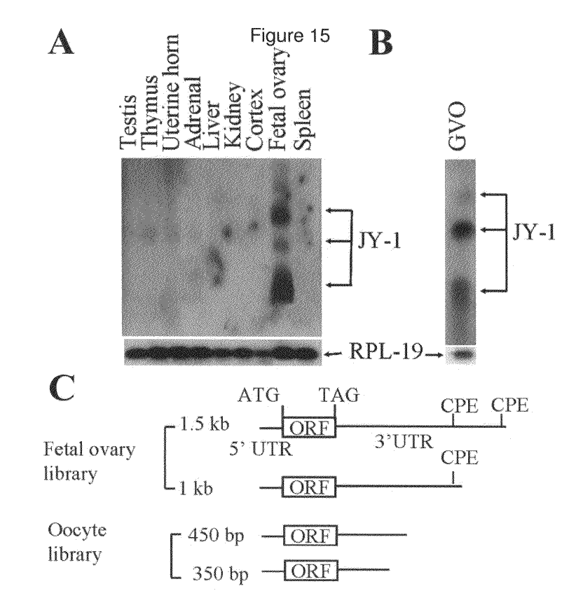

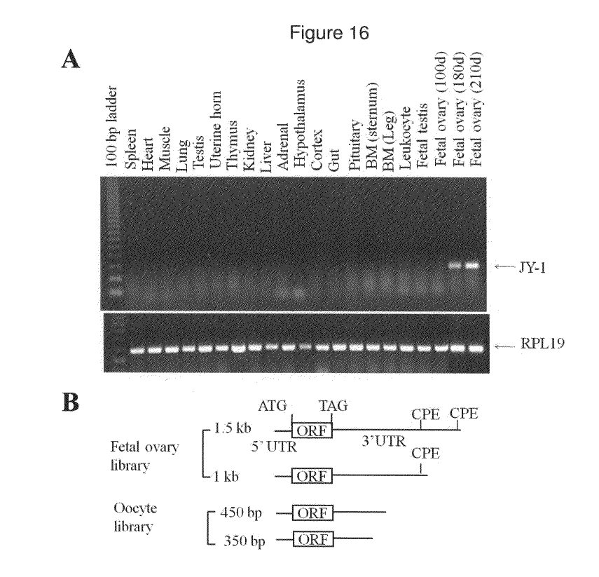

Northern analysis revealed three predominant JY-1 transcripts in RNA isolated from fetal ovaries (FIG. 5A). In particular, screening of RNA from various tissues by RT-PCR detected JY-1 mRNA in fetal ovaries collected at d 180 and 210 of gestation but not in any other tissues examined (FIGS. 15 and 16A) further demonstrating tissue specific expression of JY-1 mRNA.

Analysis of adult GV oocytes by Northern blotting confirmed the presence of three major JY-1 transcripts of different length (˜1.8 kb, 1.2 kb, and 700 bp) (FIG. 5B). The first 14 JY-1 inserts sequenced from the SMART oocyte library were small (the longest was ˜455 bp in length) and were either partial cDNAs or represent the smaller predominant or represent the smaller predominant transcript detected by Northern analysis. This oocyte library constructed using SMART technology contained mostly short cDNAs (<500 bp in length), and was not useful for cloning larger JY-1 transcripts.

Therefore, a fetal ovary conventional cDNA library in a Lambda ZAP II vector (Stratagene) was constructed using RNA isolated from a fetal ovary collected at d 210 of gestation. Then the library was screened using a 455 bp JY-1 cDNA as a probe yielding 2 additional clones containing larger inserts. One of the clones contains an insert of approximately 1.5 kb and the other clone contains an insert of approximately 1.0 kb in length. (see, SEQ ID NO:7).

Overall, three predominant transcripts of 1.5, 1.0 and 0.4 kb were detected, along with some additional minor transcripts in RNA from fetal ovaries in addition to a full-length JY-1 cDNA. One larger clone contained an insert of 1.5 kb and another clone had an insert of ˜1.0 kb in length (GenBank accession numbers are EF642496 and EF642497).

5′RACE experiments did not reveal additional 5′ expressed sequence confirming that the sequence observed at the 5′ end of exemplary JY-1 transcripts was complete. Thus, the minor differences in the length of JY-1 transcripts observed in fetal ovary versus adult GV oocytes is most likely attributed to polyadenylation status of the mRNA transcripts. Sequences upstream of the defined start AUG in four JY-1 transcripts did not contain additional in-frame start codons.

An identical open reading frame (ORF) of 255 bp encoding for a predicted protein of 84 amino acids was identified in the 4 transcripts derived from the oocyte and fetal ovary libraries (FIGS. 15A and 15B). Thus the four transcripts differed in length of the 3′ untranslated region (UTR) but had an identical 5′UTR. The AU rich putative cytoplasmic polyadenylation elements (AUUUUAAAA and UAUUUUAAUA) were also noted in the 3′ UTR of the two longest transcripts (FIG. 5B). Sequence analysis of these 2 larger JY-1 cDNAs as well as 2 original smaller cDNAs (455 bp and 355) from the oocyte library revealed that these 4 cDNA fragments represented 4 different transcripts of the JY-1 gene (FIG. 15B).

Cytoplasmic polyadenylation elements were also noted in the 3′ UTR of the two longest transcripts. Such motifs are characteristic of transcripts with potential regulation of polyadenylation, see for example, Fox et al. Genes Dev 3:2151-62 (1989); McGrew et al., Genes Dev 3:803-15 (1989); all of which are herein incorporated by reference.

### Example III

### Characterization of JY-1 Protein

When the putative amino acid sequence was analyzed using a Signal IP3 program (Nielsen et al. (1999) Protein Eng 12, 3-9, herein incorporated by reference) the program output predicted a signal peptide of 21 amino acids, indicating that JY-1 protein is likely to be secreted from the oocyte. Further, a predicted molecular weight of JY-1 was ˜9,000 Mr, but the NetOGlyc-3.1 program (Julenius et al. (2005) Glycobiology 15, 153-164) predicted two O-linked glycosylation sites in the deduced JY-1 amino acid sequence, suggesting probable glycosylation of the JY-1 protein with a correspondingly higher molecular weight.

Polyclonal antiserum was raised against recombinant JY-1 (rJY-1) protein (mature form without the signal peptide) and used in Western blot analysis to detect JY-1 protein. Immunoreactive JY-1 protein of ˜11,000 Mr and additional higher Mr bands were detected in extracts of adult GV oocytes (FIG. 5C). The polyclonal antiserum also detected the rJY-1 protein (6,700 Mr, mature form lacking the signal peptide) that was used to generate the antiserum (FIG. 5C). Preincubation of JY-1 antiserum with excess antigen (rJY-1) blocked binding of the antibody specifically to the 11,000 Mr protein and to rJY-1 protein, but not to the higher Mr bands (FIG. 5D) which represent non-specific cross reactivity. Immunoreactive JY-1 protein was detected in adult GV oocytes but not in any other cell/tissue samples examined (FIG. 5E). The 11,000 Mr JY-1 protein was not detected in GV oocytes when blots were incubated with pre-immune rabbit serum (FIG. 5F).

Publicly available databases were searched with the predicted amino acid sequence of JY-1 to identify functional domains and predict the structure of the JY-1 protein. No significant orthologs of the JY-1 protein were found in available protein databases. A putative secondary structure for JY-1 protein was predicted using the PSIPRED program (McGuffin et al. (2000) Bioinformatics 16, 404-405, herein incorporated by reference), but the inventors have not identified motifs that are indicative of functional domains using the conserved domain database (CDD) (Marchler-Bauer et al. (2002) Nucleic Acids Res 30, 281-283, herein incorporated by reference).

Similarly, pFam A and B (Bateman et al. (2000) Nucleic Acids Res 28, 263-266, herein incorporated by reference) and a PSI_BLAST search of the PDB database at NCBI (Altschul et al. (1997) Nucleic Acids Res 25, 3389-3402, herein incorporated by reference) designed to designate sequences to protein families based on homology and identify 3D structures for homology modeling were unsuccessful. Thus, the inventors conclude that JY-1 is a member of a novel protein family.

### Example IV

### Oocyte Specific Localization of JY-1 mRNA and Protein within Ovarian Follicles

The experiments described in this example demonstrate the correlation of mRNA and protein with ovarian follicales.

In situ hybridization and Immunohisochemistry Analysis. In order to determine whether JY-1 expression within the ovary was indeed oocyte-specific, in situ hybridization and immunohistochemistry experiments were performed to identify the intraovarian cell types that express JY-1 mRNA.

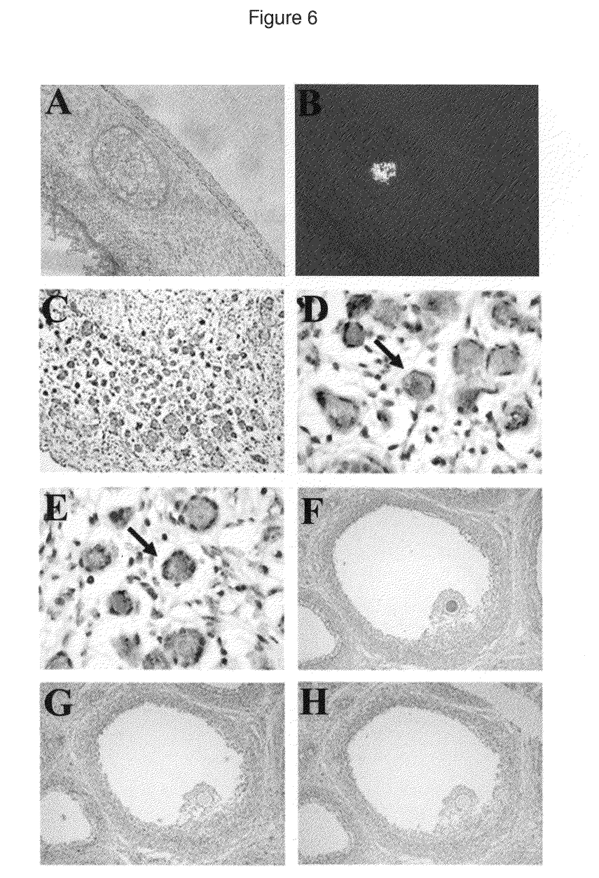

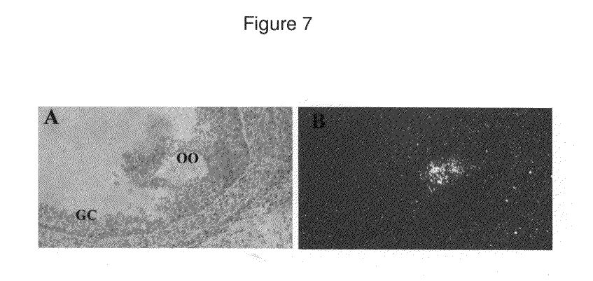

The inventors found that intraovarian expression of JY-1 mRNA and protein was restricted exclusively to oocytes. In particular, in situ hybridization localized JY-1 mRNA specifically to oocytes of preantral and antral follicles (FIG. 6A-D). No significant hybridization to somatic ovarian cell types (granulosa, theca and stroma) was noted. JY-1 protein was localized to oocytes of growing follicles at the primordial (single layer, with less than 10 flattened granulosa cells), primary (single layer with cuboidal granulosa cells) through antral follicle stages (FIG. 6A-D) in fetal ovaries collected at d 230 of gestation. Immunoreactivity was not detected when tissue sections were incubated with pre-immune rabbit IgG or when the JY-1 antibody was preabsorbed with immunogen peptide.

The results showed that intraovarian expression of JY-1 mRNA was restricted to oocytes. JY-1 mRNA was detected in oocytes of follicles from primary through Graafian stages of development. The temporal localization of JY-1 mRNA during folliculogenesis is similar to that of other key oocyte-specific (GDF-9, BMP-15) or gonad-specific (ZAR 1) genes and thus consistent with a potential involvement of JY-1 in regulation of ovarian follicle development or early embryogenesis (Wu et al., Nat Genet. 33:187-91 (2003); McGrath et al., Mol Endocrinol 9:131-6 (1995); Dube et al., Mol Endocrinol 12:1809-17 (1998); Dong et al., Nature 383:531-5 (1996); all of which are herein incorporated by reference).

### Example V

### Effect of Recombinant JY-1 Protein on Cell Number and Production of Estradiol and Progesterone by Cultured Granulosa Cells

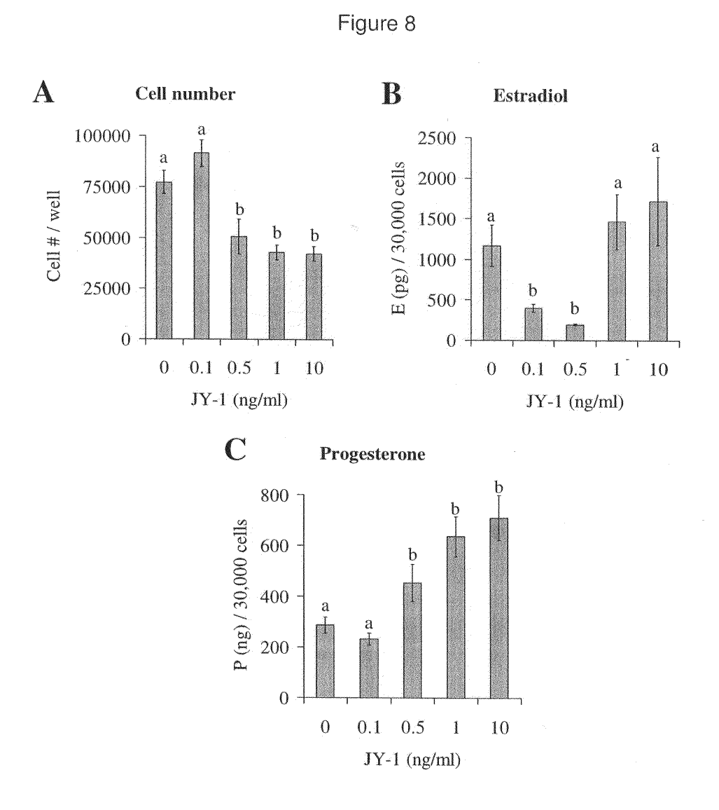

A rJY-1 protein was expressed and utilized to test the ability of JY-1 to regulate bovine granulosa cell proliferation and steroidogenesis. Addition of rJY-1 to cultured granulosa cells inhibited the FSH induced increase in granulosa cell numbers at the 0.5 ng/ml dose (P<0.05) and the response was maximal at 1 and 10 ng/ml doses (P<0.05, FIG. 8A). Addition of rJY-1 at 0.1 ng/ml had no effect on granulosa cell numbers (FIG. 8A). However, in vitro production of estradiol (E) was inhibited 2 fold in FSH supplemented granulosa cells (P<0.05) treated with the 0.1 ng/ml rJY-1 dose where significant effects on granulosa cell numbers were not observed (FIG. 8B). Further, the inhibitory effect on E production was maximal at 0.5 ng/ml rJY-1 and supplementation with 1 and 10 ng/ml rJY-1 did not inhibit E production. In contrast, addition of rJY-1 increased production of progesterone (P) in a dose dependent manner (P<0.01) and the response was maximal at 1 and 10 ng/ml rJY-1 (FIG. 8C). Even though total cell numbers were decreased by 50% in response to treatment with 1 and 10 ng/ml rJY-1, P production was doubled compared to cells cultured without rJY-1 (FIGS. 8A and C). No effects of rJY-1 on granulosa cell numbers or E and P production were observed for granulosa cells cultured in the absence of FSH.

### Example VI

### Reverse Transcription Polymerase Chain Reaction Analysis of Tissue Specific Expression

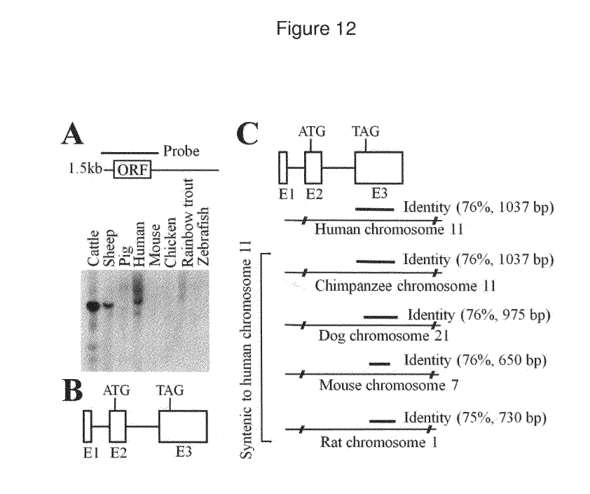

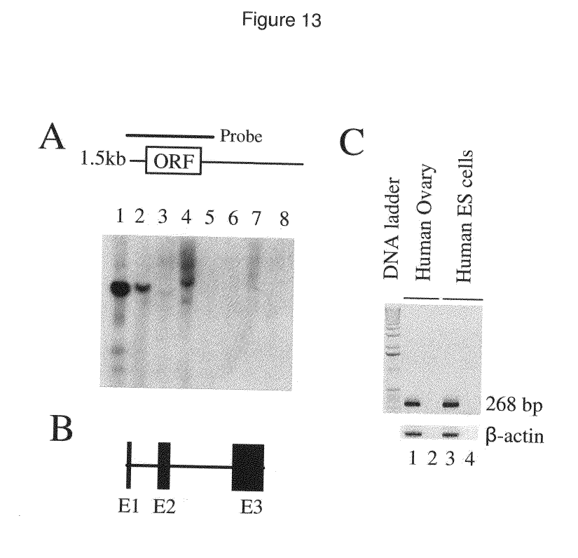

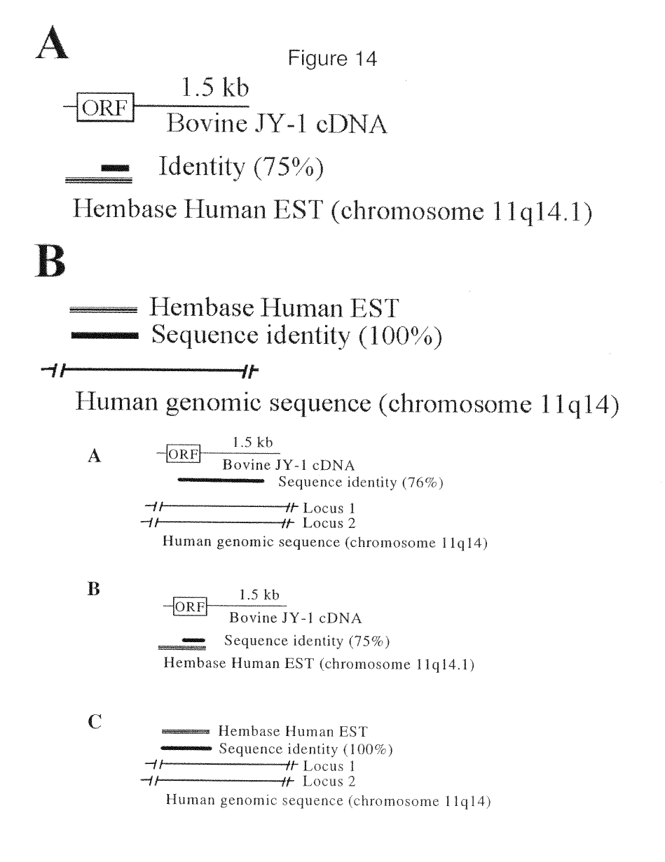

Reverse transcription polymerase chain reaction (RT-PCR) analysis indicated that JY-1 mRNA was expressed in a tissue-specific fashion. Expression of JY-1 mRNA was detected in fetal ovaries (an enriched source of oocytes) and in isolated oocytes collected from adult animals. Expression of JY-1 mRNA was not detected in RNA isolated from over 18 different tissues, including testis). Amplification of the housekeeping gene ribosomal protein L-19 (RPL-19) was used as a positive control to verify that cDNA synthesis was successful. The inventors believe that JY-1 mRNA expression is gonad-specific ovarian expression (FIGS. 12A and 13A).

### Example VII

### Quantitative Real-Time PCR Analysis

Quantitative real-time PCR results indicate that the abundance of JY-1 mRNA in fetal ovaries is developmentally regulated. JY-1 mRNA was readily detected in fetal ovaries collected at 180 and 210 d of gestation but not in fetal ovaries collected at d 100 of gestation.

For quantitative analysis of JY-1 mRNA expression in ovaries during fetal development, quantitative real time PCR was performed on total RNA isolated from fetal ovaries of different gestation stages. Two μg of each RNA sample were converted to cDNA using Superscript II reverse transcriptase (Invitrogen) as recommended by the manufacturer. Quantitative PCR was performed in duplicate for each cDNA sample on an ABI 7000 Sequence Detection System (Applied Biosystems, Foster City, Calif.) using the default 2-step amplification procedure and 2×SYBR Green Master Mix (Applied Biosystems Foster City, Calif.) in 50 μl reaction volume with 300 nm of each primer and cDNA derived from 0.2 μg of total RNA. The primers for JY-1 amplification were: 5′-TTGGAACTTCCATGGACGACC-3′ (SEQ ID NO: 30) (forward) and 5′-ATTTGCTGGTGATCCCAAGAG-3′ (SEQ ID NO: 31) (reverse). As an endogenous control for normalization, β-actin gene was also amplified for each sample using the following primer pair: 5′-CGCCATGGATGATGATATTAC-3′ (SEQ ID NO:46) (forward) and 5′-AAGCCGGCCTTGCACAT-3′(SEQ ID NO:47) (reverse).

For relative quantitation, standard curves for both JY-1 and β-actin were constructed using 10-fold serial dilutions of the corresponding purified PCR product. Standard curves were run on the same plate as the samples. Threshold lines were adjusted to intersect amplification lines in the linear portion of the amplification curve and cycles to threshold (Cₜ) were recorded. The Cₜ value of JY-1 was divided by the Cₜ value of β-actin for each sample for normalization and the JY-1 expression levels of the samples are expressed as relative to the sample with the lowest expression level.

The β-actin gene was used as an endogenous control to correct for RNA variations among samples. Expression levels of JY-1 transcripts in ovaries at various stages of fetal development were expressed relative to fetal ovary at 95 d of gestation (lowest expression detected). The results demonstrate that JY-1 mRNA is barely detectable in ovaries collected during early stages of gestation and mRNA abundance is increased dramatically in ovaries collected at later stages of gestation. This temporal pattern for JY-1 mRNA abundance is consistent with potential expression beginning at the primary follicle stage as reported for other key oocyte-expressed genes with important regulatory roles in folliculogenesis or early development.

### Example VIII

### Immunolocalization of JY-1 Protein and Western Blot Analysis

The Polyclonal antiserum generated against a 20 amino acid synthetic peptide corresponding to a portion of the carboxy-terminus of the predicted amino acid sequence of bovine JY-1 (C55-A74) was used herein. This antiserum was used in the Western blot and immunocytochemistry experiments that established JY-1 mRNA was translated into a functional protein in bovine oocytes and determined intraovarian localization of JY-1 protein during folliculogenesis. Peptide synthesis, conjugation to keyhole limphet hemocyanin, immunization, and immunoaffinity purification was conducted commercially by Bethyl Laboratories.

For immunocytochemistry analysis, samples of fetal and adult ovaries were collected, fixed in neutral buffered formalin, dehydrated and embedded in paraffin according to standard procedures. Approximately 6 μM sections were deparaffinized, rehydrated, and treated with 3% hydrogen peroxide to block endogenous peroxidase activity. Non specific binding is blocked by incubation of sections in 4% normal goat serum in phosphate buffered saline. Sections were then treated with approximately a 1/1000 dilution (1 μg/ml) of affinity purified anti JY-1 IgG, 1 μg/ml preimmune IgG or 1 μg/ml affinity purified anti JY-1 IgG preincubated with 100 fold excess immunogen peptide overnight at 4° C. prior to incubation with sections. After washing and incubation with appropriate secondary antibody, positive staining was visualized using the Streptavidin-biotin peroxidase system and Texas Red detection system according to manufacturer's instructions (Vector labs), followed by counterstaining with hematoxylin and eosin.

Western blot analysis was conducted on extracts of fetal ovaries collected at approximately 140, 210, 250 and 260 d of gestation. Tissues were collected at a local abattoir and frozen in liquid nitrogen until processed for Western analysis. Tissues were homogenized using a polytron homogenizer (Fisher Scientific, Chicago, Ill.) in 10 mM calcium chloride; 0.25% Triton X-100. The homogenates were then centrifuged at 9,000 g for 30 min at 4° C. The supernatants were collected and frozen at −20° C. until use. Extracts (20-30 μg) were separated on SDS polyacrylamide gels and electrophoretically transferred to PVDF membranes. Western blot procedures were conducted according to manufacturer's instructions (femto-Lucent detection system; Geno Technology, Inc). Membranes were blocked, washed, incubated in a 1/1000 dilution of JY-1 IgG, preimmune IgG, or in absence of primary antibody overnight at 4° C. prior to secondary antibody incubation and chemiluminescent detection.

The results indicate that JY-1 protein is localized to oocytes of follicles at the primary through Graafian follicle stages, similar to the localization pattern observed for JY-1 mRNA. Pre-absorption of the JY-1 antisera with immunogen peptide blocked binding of the antiserum to the oocyte. Preliminary results from Western blot experiments indicate the JY-1 antiserum recognizes a protein of the predicted approximate Mr present within extracts of bovine fetal ovaries collected during late gestation (d 260), but not in samples collected earlier in gestation. The immunoreactive protein of interest was not detected when a duplicate blot was incubated with preimmune serum. The results to date support existence of immunoreactive JY-1 protein specifically within bovine oocytes

### Example IX

### Bioinformatics Analysis

Using the longest bovine JY-1 cDNA sequence (1.5 kb), a search was conducted for homologous sequences in the Genbank database. One significant match was identified in the nonredundant (nr) database was with a human genomic fragment from chromosome 11. About 500 bp of sequence in the 3′UTR plus a portion of the coding region of the JY-1 cDNA shares approximately 80% similarity with this human genomic sequence. This level of similarity is common between bovine and human species of a gene. By searching another Genbank database, which contains all high throughput draft genomic sequences (htgs), a genomic sequence from mouse chromosome 7 (syntenic with human chromosome 11) was found that has partial sequence similarity with the bovine JY-1 cDNA. The region of sequence similarity is limited to about 400 bp of sequence in the 3′UTR of the bovine cDNA.

The nucleotide sequence of bovine JY-1 does not map to any of the approximately 24,000 bovine unique gene clusters identified to data (TIGR release 7.0; www.tigr.org). A putative secondary structure for bovine JY-1 has been established using the PSIPRED program (McGuffin et al., The PSIPRED protein structure prediction server, Bioinformatics 16:404-5 (2000), herein incorporated by reference), but nonsequence motifs have been identified in the JY-1 predicted amino acid sequence to date that are indicative of functional domains using CDD and other programs (Marchler et al., CDD: a database of conserved domain alignments with links to domain three-dimensional structure, Nucleic Acids Res 30:281-3 (2002), herein incorporated by reference). In addition, searches of numerous databases, such as pFam A and B (Bateman et al. The Pfam protein families database, Nucleic Acids Res 28:263-6 (2000), herein incorporated by reference) and a PSI_BLAST search of the PDB database at NCBI (Altschul et al., Gapped BLAST and PSI-BLAST: a new generation of protein database search programs, Nucleic Acids Res 25:3389-402 (1997), herein incorporated by reference) designed to designate sequences to protein families based on homology and identify known 3D structures for homology modeling were unsuccessful.

### Example X

### JY-1 Gene Structure

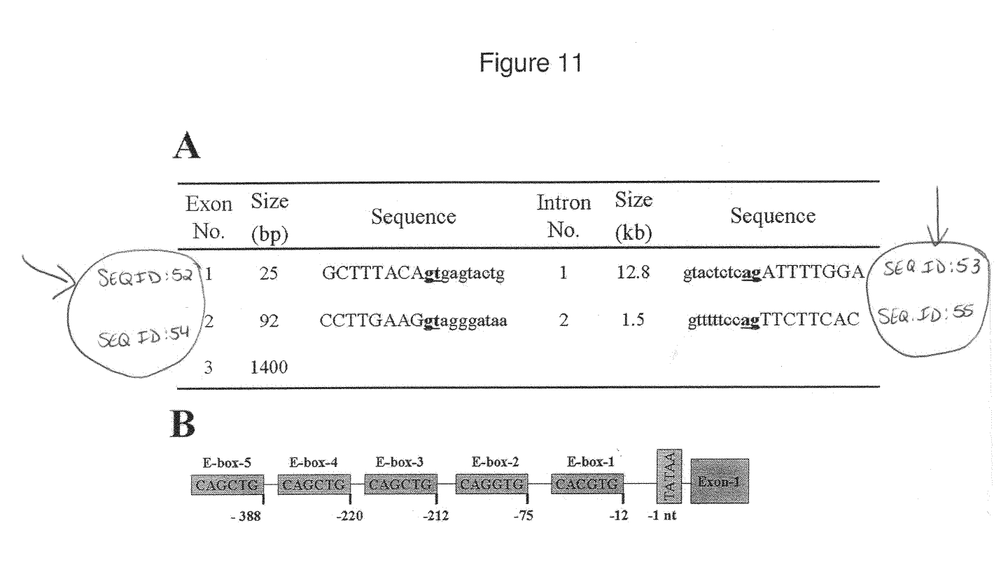

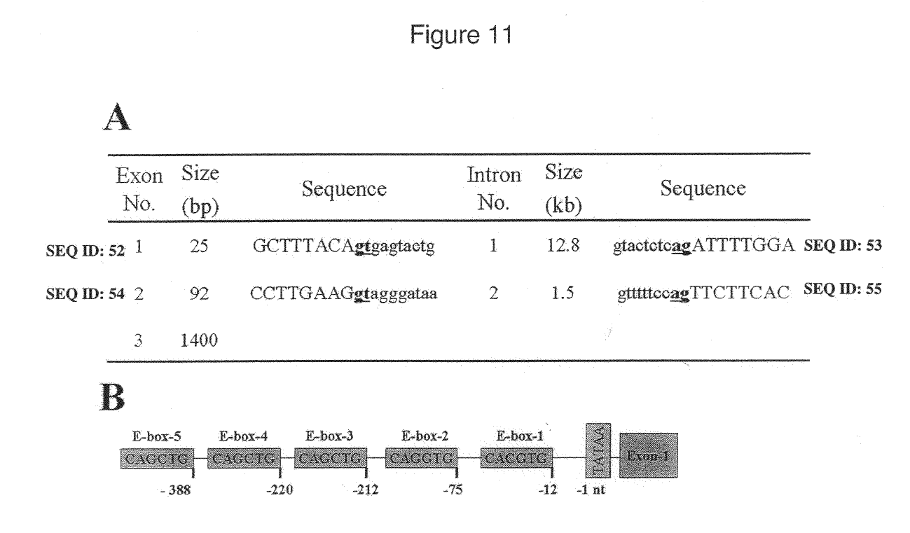

In order to characterize and study JY-1 transcriptional regulation, a bovine genomic DNA library was screened with a 5′probe generated from the JY-1 cDNA and two genomic clones characterized. The objectives were to determine the gene structure for JY-1 and to isolate 5′ flanking regions of the JY-1 gene and determine cis elements (sequence motifs) and trans acting factors (transcription factors) that confer oocyte-specific expression. Two genomic clones characterized contained exons 2 and 3, intron 2 and a portion of introns 1 and 3. Exon sequence obtained to date corresponds closely to cDNA sequence obtained for JY-1. A complete single pass sequence for the region of the JY-1 gene corresponding to the last 500 bp of intron 1 through the first 2.9 kb of kb of intron 3 was obtained (FIG. 11A).

A 5′-flanking sequence of the JY-1 gene was also sequenced and analyzed in order to identify putative cis-elements that may confer tissue/cell specific expression of JY-1. After sequencing, five putative E-boxes (canonical sequence CANNTG; known to mediate oocyte-specific expression (Yan et al. (2006) Biol Reprod 74, 999-1006; herein incorporated by reference) in other species were identified within 500 bp of the 5′flanking sequence of the bovine JY-1 gene (FIG. 11B).

### Example XI

### Quantification of JY-1 mRNA During the Oocyte-to-Embryo Transition and Effect of JY-1 Knockdown on Early Embryonic Development

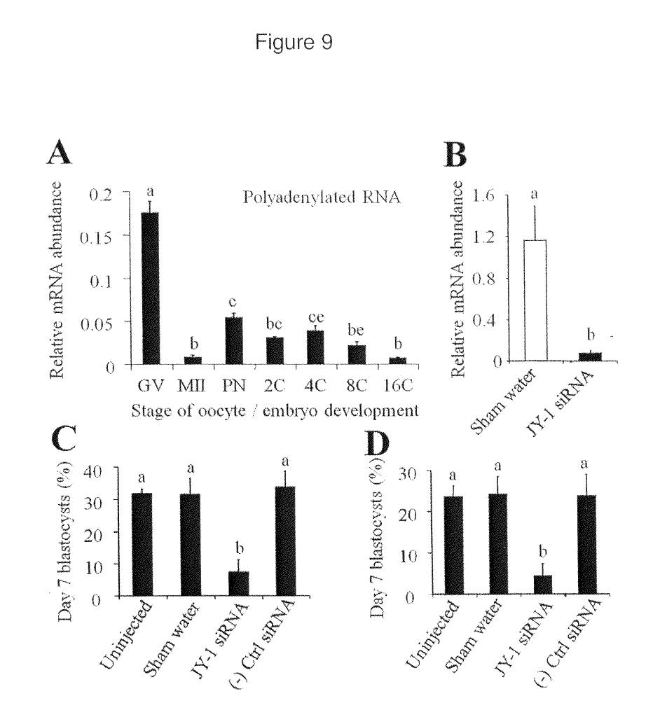

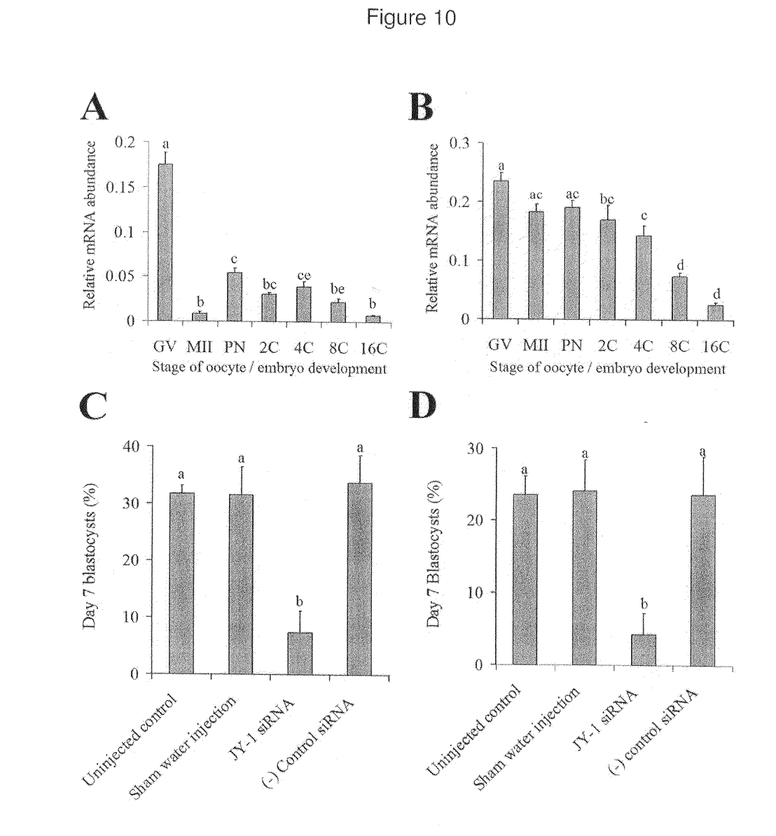

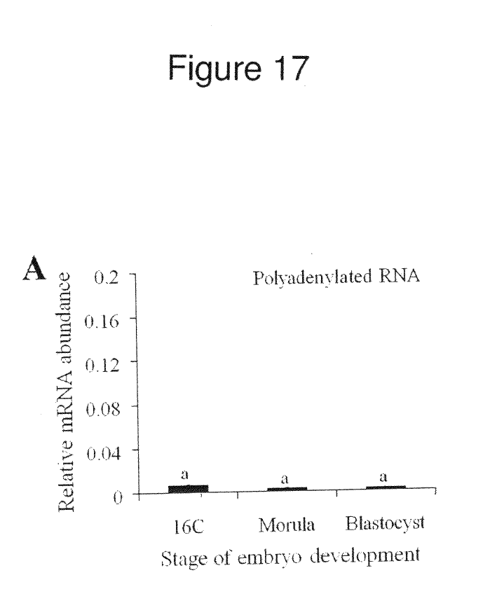

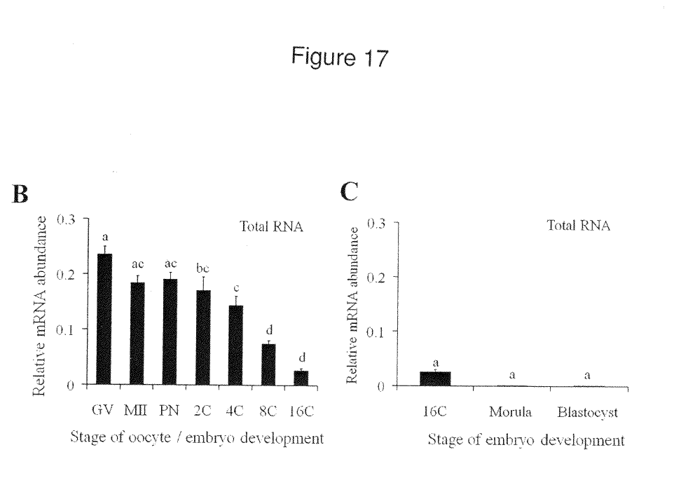

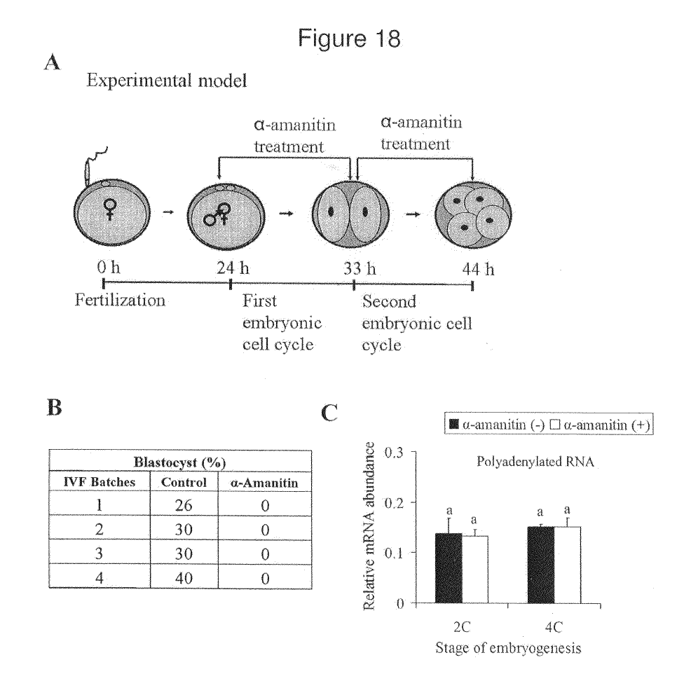

Because the inventors observed oocyte-specific localization of JY-1 mRNA and protein, the inventors hypothesized that JY-1 mRNA is regulated during meiotic maturation and early embryonic development. Temporal changes in abundance of polyadenylated versus total JY-1 transcripts during early development were characterized by quantitative real-time PCR. Abundance of polyadenylated JY-1 transcripts (cDNAs synthesized from oligo dT primers) decreased during meiotic maturation (P<0.0001), were increased (P<0.05) at the pronuclear and 4-cell stages relative to the metaphase II (MII) stage, and then decreased to nearly undetectable levels after the 16-cell stage of embryo development (FIG. 9A, FIG. 10A). In contrast, amount of total JY-1 transcripts (cDNAs synthesized from random hexamers) gradually decreased from GV through 16-cell stages to nearly undetectable levels thereafter (FIGS. 17B and 17C). The difference in abundance of polyadenylated versus total transcripts is probably the result of JY-1 mRNA deadenylation. Further, results of embryo culture experiments in the presence of the transcription inhibitor α-amanitin suggest that the JY-1 gene is not transcribed during the first and second embryonic cell cycles (FIG. 18) and thus the JY-1 mRNA detected in early bovine embryos is maternal/oocyte derived.

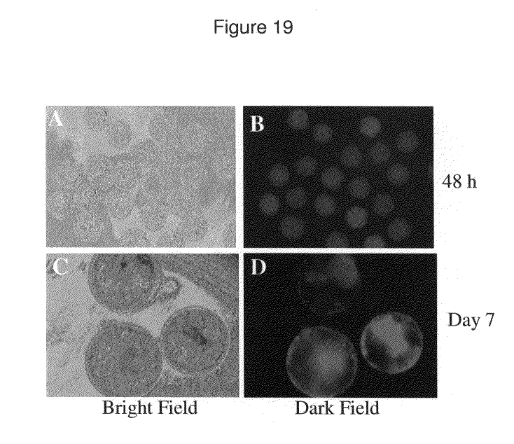

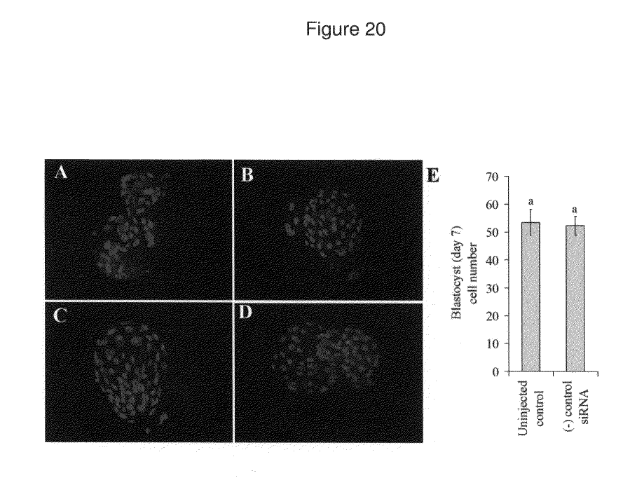

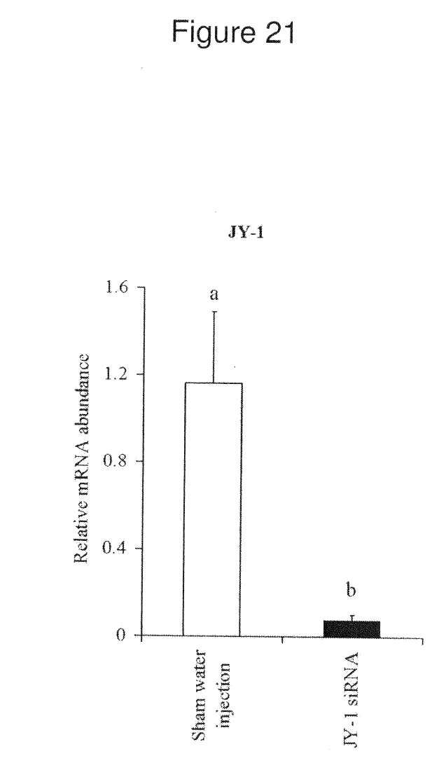

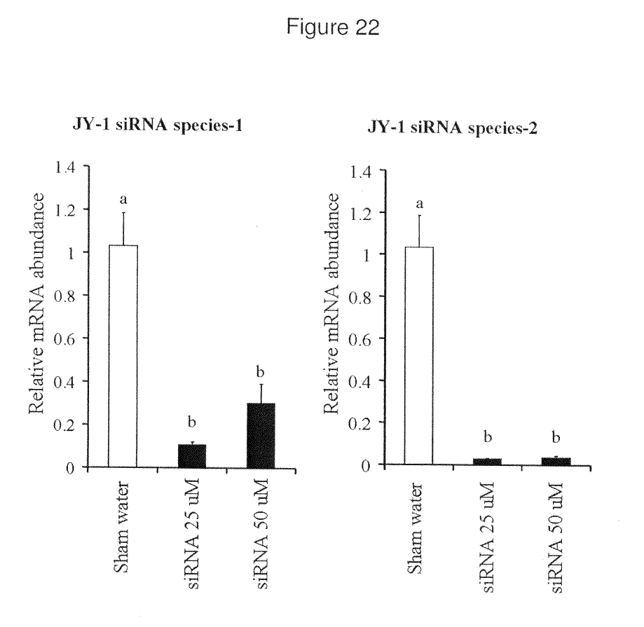

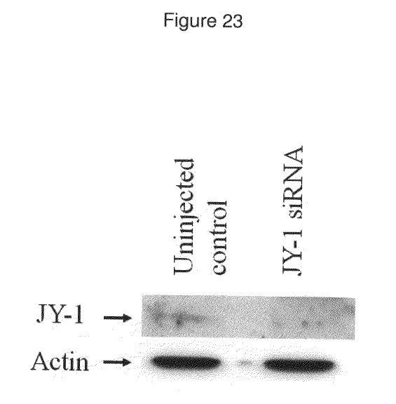

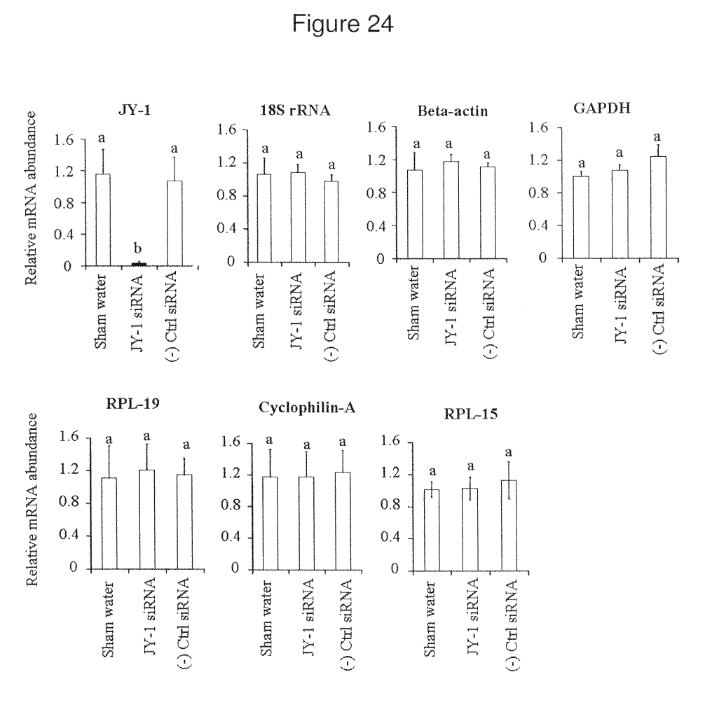

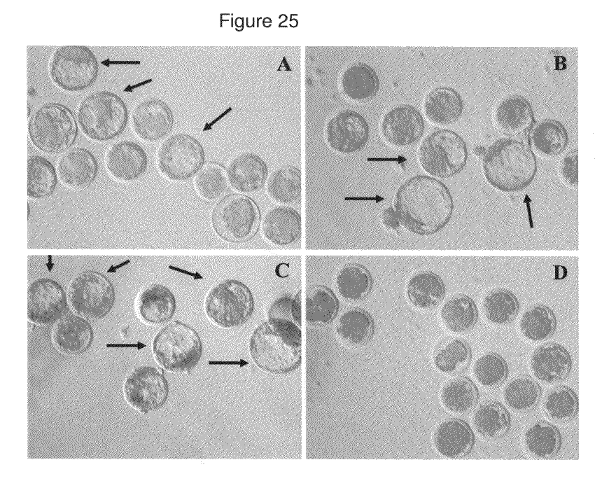

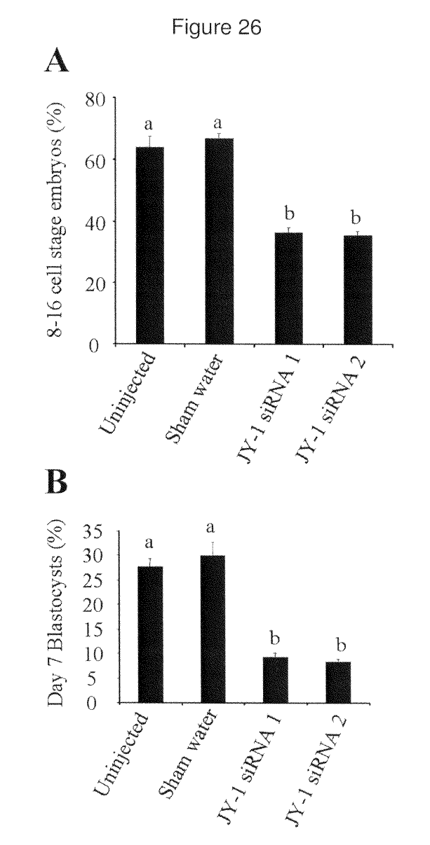

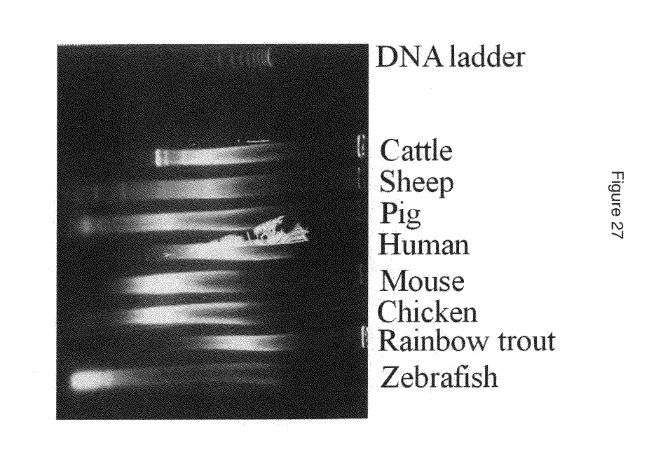

To test the requirement of JY-1 during early embryonic development, the inventors validated procedures for siRNA mediated gene silencing in bovine embryos. Multiple siRNA species were tested for efficacy and specificity of JY-1 mRNA knockdown via microinjection into MII oocytes followed by parthenogenetic activation. Parthenogenesis was utilized as a model to test the efficacy of JY-1 knockdown in embryos because it is easier to manipulate and allows for cumulus cell removal and siRNA injection earlier in development. The two most effective siRNA species (species 1 and 2, at 25 μM concentration) reduced JY-1 mRNA abundance by approximately 90% in 4-cell stage embryos and a cocktail of both siRNAs reduced JY-1 mRNA abundance by approximately 95% in 2-cell embryos (FIG. 10B). JY-1 siRNA cocktail specifically reduced JY-1 mRNA in 4-cell embryos with no effect on RNA abundance for 6 control genes examined (FIG. 23). Specificity of the JY-1 siRNA was further confirmed via measurement of JY-1 protein abundance in 8-16 cell embryos. JY-1 siRNA specifically reduced JY-1 protein to undetectable levels compared to uninjected control embryos (FIG. 19).

JY-1 siRNA cocktail injection strikingly decreased the proportion of parthenogenetic embryos developing to the blastocyst stage (7.4%) relative to uninjected (31.7%), sham injected (31.5%) and negative control siRNA injected (33.7%) embryos (P<0.05, FIG. 9C). Cleavage rates of embryos were not different between the groups. Similarly, JY-1 siRNA cocktail injection into IVF embryos did not affect the cleavage rates but dramatically reduced the proportion of IVF embryos developing to the blastocyst stage (4.2%) relative to uninjected (23.5%), sham injected (24.1%) and negative control siRNA injected (23.6%) embryos (P<0.01, FIG. 9D). To further ensure the specificity of JY-1 siRNA in inhibiting embryonic development, experiments were repeated with the individual siRNAs injected separately. A reduction in the proportion of IVF embryos developing to the 8-16 cell stage (P<0.0001) and proportion of embryos developing to the blastocyst stage (P<0.0001) relative to uninjected and sham injected controls was noted after injection of each siRNA individually.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, reproductive physiology or related fields are intended to be within the scope of the following claims.

1. (canceled)

2. (canceled)

3. (canceled)

4. (canceled)

5. (canceled)

6. (canceled)

7. (canceled)

8. (canceled)

9. (canceled)

10. (canceled)

11. An isolated polypeptide encoded by a nucleic acid selected from the group consisting of SEQ ID NOs: 1, 7, 9 and variants thereof at least 75% identical to SEQ ID NOs: 1, 7 and 9.

12. The polypeptide of claim 11, wherein said protein is at least 85% identical to SEQ ID NOs: 1, 7 and 9.

13. The polypeptide of claim 11, wherein said protein is at least 95% identical to SEQ ID NOs:1, 7 and 9.

14. A purified antibody that binds specifically to the isolated polypeptide of claim 11.

15. A composition comprising a nucleic acid sequence that inhibits the binding of at least a portion of a nucleic acid selected from the group consisting of SEQ ID NOs: 1, 7 and 9 to it's complementary sequence.

16. The composition of claim 15, wherein said nucleic acid sequence is selected from the group consisting of SEQ ID NO:32-38.

17. A polynucleotide sequence comprising at least fifteen nucleotides capable of hybridizing under stringent conditions to an isolated nucleic acid sequence selected from the group consisting of SEQ ID NOs: 1, 7 and 9.

18. A method comprising:
a) providing,
i) an inhibitory JY-1 RNA; and
ii) a host target cell expressing a sense JY-1 nucleotide sequence; and
b) introducing said inhibitory JY-1 RNA into said host target cell under conditions such that said sense JY-1 nucleotide sequence expression levels are reduced.

19. The method of claim 18, wherein said inhibitory JY-1 RNA is an siRNA.

20. The method of claim 19, wherein said siRNA is selected from the group consisting of SEQ ID NOs:32-35.

21. The method of claim 18, wherein said inhibitory JY-1 RNA further comprises an RNA expression vector.

22. (canceled)

23. (canceled)

24. (canceled)

25. (canceled)

26. A vaccine comprising a JY-1 polypeptide selected from the group consisting of SEQ ID NOs: 1, 7, 9, and a variant thereof.

27. A method for altering in vivo fertility, comprising:
a) providing:
i) a female subject, wherein said subject comprises a cell selected from the group consisting of an oocyte and an embryonic cell; and
ii) a composition comprising a polypeptide, wherein said polypeptide is selected from the group consisting of SEQ ID NO:08 and a variant of SEQ ID NO:08, and
b) injecting said female subject with said composition under conditions that alter in vivo fertility.

28. The method of claim 27, wherein said composition increases fertility.

29. The method of claim 27, wherein said alteration of fertility comprises enhancing oocyte development.

30. The method of claim 27, wherein said alteration of fertility is decreasing fertility.

31. The method of claim 27, further comprising, providing, an agent for altering fertility and injecting said agent into said subject.

32. The method of claim 31, wherein said agent is selected from the group consisting of gonadotropin hormone, chorionic gonadotropin hormone, luteinizing hormone, growth hormone, follicle stimulating hormone, steroidogenic acute regulatory protein (StAR), and Cocaine- and Amphetamine-Regulated Transcript (CART).

33. The method of claim 27, wherein said subject is selected from the group consisting of monoovulatory species.

34. The method of claim 27, wherein said subject is selected from the group consisting of human, non-human primate, cattle, bison, buffalo, water buffalo, African buffalo, zebu, banteng, gaur, yak, antelope, gazelle, reindeer, moose, giraffe, bactrian camel, dromedary camel, camelid, deer, elk, caribou, swine, goat, sheep, big-horn sheep, horse, pony, donkey, zebra, mule, llama, alpaca, vicufia, guanaco, and hybrids thereof.

35. The method of claim 27, wherein said subject is selected from the group consisting of Bovidae, Homimidae, Salmonidae, and Cyprimidae.

36. The method of claim 27, wherein said subject is selected from the group consisting of mouse, chicken, rainbow trout, zebrafish, human, bovine, equine, porcine, ovine, elk, and bison.

37. The method of claim 27, wherein said subject is selected from the group consisting of bovine, human, ovine, equine, porcine and caprine.

38. The method of claim 27, wherein said variant is selected from the group consisting of bovine, human, ovine, equine, porcine and caprine.

39. A method for increasing in vitro fertility, comprising:
a) providing:
i) an oocyte cell;
ii) oocyte culture medium; and
iii) a composition comprising a polypeptide, wherein said polypeptide is selected from the group consisting of SEQ ID NO:08 and a variant of SEQ ID NO:08, and;
b) adding said oocyte cell and said composition to said culture medium; and
c) culturing said oocyte cell under conditions that said oocyte cell increases in fertility.

40. The method of claim 39, wherein said culturing generates an oocyte expressing a maturation marker.

41. The method of claim 39, wherein said culturing generates an oocyte decreasing a developmental marker.

42. The method of claim 39, wherein said culturing alters a marker selected from the group consisting of a Zygote arrest 1 (Zar1), a growth/differentiation factor-9, and a bone morphogenetic protein 15.

43. A method for alteration of in vitro fertility, comprising:
a) providing:
i) a target cell, wherein said target cell is an embryonic cell; and
ii) a composition comprising a polypeptide, wherein said polypeptide is selected from the group consisting of SEQ ID NO:08, a variants of SEQ ID NO:08; and
b) culturing said embryonic cell with said composition under conditions that alter embryonic cell development.

44. The method of claim 43, wherein said culturing generates an embryonic cell in a blastocoel stage.

45. The method of claim 43, further comprising providing a host.

46. The method of claim 45, further comprising transplanting said cultured embryonic cell into said host.

47. The method of claim 45, wherein said host is selected from the group consisting of bovine, human, ovine, equine, porcine and caprine.

48. The method of claim 43, wherein said composition further comprises at least one growth factor.

49. The method of claim 48, wherein said growth factor is selected from the group consisting of stem cell factor, Fms-like Tyrosine Kinase-3, and thrombopoietin.

50. (canceled)

51. (canceled)

52. A kit comprising a JY-1 antibody and instructions for use.

53. The kit of claim 52, further comprising a JY-1 siRNA cocktail.

54. The kit of claim 52, further comprising a rJY-1.
